SEQUENCE LISTING (6 sequences)

SEQ ID NO: 1 - DNA, 40 bp, Artificial Sequence
  Description of Artificial Sequence Synthetic oligonucleotide
cccccccccccccccccccccccccccccccccccccccc

SEQ ID NO: 2 - DNA, 40 bp, Artificial Sequence
  Description of Artificial Sequence Synthetic oligonucleotide
acacacacacacacacacacacacacacacacacacacac

SEQ ID NO: 3 - DNA, 21 bp, Artificial Sequence
  Description of Artificial Sequence Synthetic oligonucleotide
ggaucuuauuucuucggagtt

SEQ ID NO: 4 - DNA, 21 bp, Artificial Sequence
  Description of Artificial Sequence Synthetic oligonucleotide
ttccuagaauaaagaagccuc

SEQ ID NO: 5 - DNA, 21 bp, Artificial Sequence
  Description of Artificial Sequence Synthetic oligonucleotide
gcaauugaggagugccugatt

SEQ ID NO: 6 - DNA, 21 bp, Artificial Sequence
  Description of Artificial Sequence Synthetic oligonucleotide
ttcguuaacuccucacggacu

(19) United States
(12) Patent Application Publication
(10) Pub. No.: US 2012/0070417 A1
(43) Pub. Date: Mar. 22, 2012

(54) ANTI-INFLUENZA FORMULATIONS AND METHODS

(75) Inventors: Richard Batycky, Newton, MA (US); David L. Hava, Natick, MA (US); Robert W. Clarke, Medfield, MA (US); Michal M. Lipp, Framingham, MA (US)

(73) Assignee: PULMATRIX, INC., LEXINGTON, MA (US)

(21) Appl. No.: 13/259,659

(22) PCT Filed: Mar. 26, 2010
(86) PCT No.: PCT/US10/28900

§ 371 Date: Nov. 29, 2011

Related U.S. Application Data
(60) Provisional application No. 61/163,763, filed on Mar. 26, 2009.
(60) Provisional application No. 61/255,764, filed on Oct. 28, 2009.

(51) Int. Cl. A61K 33/14; A61K 38/21; A61K 31/19; A61K 31/715; A61P 31/16; A61K 31/7105; A61K 31/351; A61K 31/195; A61K 31/245; A61K 38/47; A61K 31/713

(52) U.S. Cl. 424/85.7; 424/678; 424/94.61; 424/94.3; 514/557; 514/54; 514/44 A; 514/44 R; 514/459; 514/563; 514/537

(57) ABSTRACT

The invention relates to pharmaceutical compositions that contain a calcium salt as an active ingredient and also comprise another anti-influenza agent, and to methods for treating or preventing influenza virus infection.

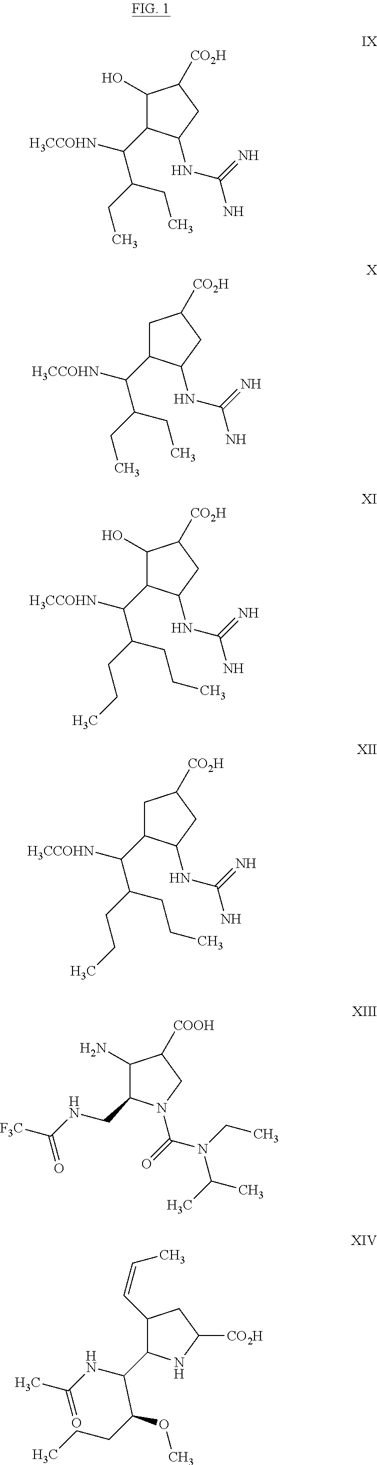

### RELATED APPLICATIONS

This application claims the benefit of U.S. Provisional Application No. 61/255,764, filed on Oct. 28, 2009 and U.S. Provisional Application No. 61/163,763, filed on Mar. 26, 2009. The entire teachings of the above applications are incorporated herein by reference.

### BACKGROUND OF THE INVENTION

Influenza, commonly known as flu, is an infectious disease of birds and mammals caused by an RNA virus of the family Orthomyxoviridae (the influenza viruses). In humans, common symptoms of influenza infection are fever, sore throat, muscle pains, severe headache, coughing, and weakness and fatigue. Merck Manual Home Edition. “Influenza: Viral Infections” In more serious cases, influenza causes pneumonia, which can be fatal, particularly in young children and the elderly. Sometimes confused with the common cold, influenza is a much more severe disease and is caused by a different type of virus. Lancet Infect Dis 5 (11): 718-25 (2005).

Typically, influenza is transmitted from infected mammals through airborne droplets and aerosols containing the virus, and from infected birds through their droppings. Influenza can also be transmitted by saliva, nasal secretions, feces and blood. Infections occur through contact with these bodily fluids or with contaminated surfaces.

Influenza spreads around the world in seasonal epidemics, killing millions of people in pandemic years and hundreds of thousands in non-pandemic years. Often, new strains of the influenza virus result from the mutation of an existing flu virus in animal species which become infectious to humans. Since it first killed humans in Asia in the 1990s, a deadly avian strain of H5N1 has posed the greatest risk for a new influenza pandemic; however, this virus has not mutated to spread easily between people.

Vaccinations against influenza are most commonly given to high-risk humans in industrialized countries and to farmed poultry. WHO weekly Epidemiological Record 19 Aug. 2005, vol. 80, 33, pp. 277-288; Poult Sci 77 (8): 1143-5 (1998). The most common human vaccine is the trivalent influenza vaccine that contains purified and inactivated material from three viral strains. Typically this vaccine includes material from two influenza A virus subtypes and one influenza B virus strain. Thorax 57 Suppl 2: 1124-1130. A vaccine formulated for one year may be ineffective in the following year, since the influenza virus changes rapidly over time and different strains become dominant.

In cases where vaccines are ineffective or the flu patient was never vaccinated, antiviral drugs (e.g., oseltamivir) can be used to treat influenza, with neuraminidase inhibitors being particularly effective. Recently, however, H1N1 and H3N2 Influenza A viruses that are resistant to neuraminidase inhibitors have spread rapidly around the world. The emergence of drug resistant influenza viruses (e.g., oseltamivir resistance) has highlighted a need for the development of new therapies capable of treating influenza. The emergence of multiple influenza strains during a single flu season, and the inefficiency of confirming which strain patients are infected with, has highlighted a need for treatment of influenza-like illness, whereby patients exhibiting influenza-like symptoms may be treated without waiting for confirmation of influenza infection.

### SUMMARY OF THE INVENTION

The invention relates to a pharmaceutical composition that comprises a calcium salt as an active ingredient and further comprises another anti-influenza agent. The pharmaceutical compositions of the invention are suitable for administration to the respiratory tract, for example, by inhalation (e.g., as an aerosol). In a particular embodiment, the pharmaceutical composition comprises a calcium salt, such as calcium chloride, calcium lactate, calcium citrate, calcium sulfate or the like, and an influenza neuraminidase inhibitor, such as zanamivir.

The invention relates to methods for treatment, prophylaxis or reducing spread of influenza or influenza-like illness that comprise administering an effective amount of a pharmaceutical composition to an individual. The invention also relates to methods for treatment, prophylaxis or reducing spread of influenza or influenza-like illness that comprise administering an effective amount of a salt formulation (e.g., a calcium salt formulation) and an effective amount of an anti-influenza agent to a person in need thereof, wherein the salt formulation is administered to the respiratory tract. For example, an individual suspected of having influenza, with confirmed influenza, at risk for influenza, or with influenza-like illness can be treated in accordance with the methods described herein.

The invention also relates to the use of a salt formulation, as described herein, and an anti-influenza agent for the manufacture of a medicament for the treatment, prophylaxis or reducing spread of influenza or influenza-like illness.

The invention also relates to methods for treatment, prophylaxis or reducing spread of influenza-like illness that comprise administering an effective amount of a salt formulation (e.g., a calcium salt formulation), wherein the salt formulation is administered to the respiratory tract. In a particular embodiment, the influenza-like illness is parainfluenza.

### BRIEF DESCRIPTION OF THE DRAWINGS

FIG. 1 shows the structures of the influenza virus neuraminidase inhibitors peramivir (IX), BCX-187 (X), BCX-1898 (XI), BCX-1923 (XII), A-192558 (XIII), and A-315675 (XIV). See, Smee et al., Antimicrob. Agents Cheother., 45:743-748 (2001) regarding IX-XII. See, Wang et al., J. Med. Chem., 44:1192-1201 (2001) regarding XIII; and Hanessian et al., J. Am. Chem. Soc., 124:4716-4721 (2002) regarding XIV.

FIG. 2 shows the structures of the influenza virus neuraminidase inhibitors R-125489 (XV), CS-8958 (XVI) and of dimeric zanamivir (XVII). See, Yamashita et al., Antimicrob. Agents Cheother., 53:186-192 (2009) regarding XV and XVI; and Macdonald et al., Antimicrob. Agents Cheother., 48:4542-4549 (2004) regarding XVII.

FIG. 3 shows the structures of the influenza virus M2 channel inhibitors amantadine (XVIII), rimantadine (XIX), spiro[cyclpropane-1,2′-adamantan]-2-amine (XX), spiro[pyrrolidine-2,2′-adamantane] (XXI), spiro[piperidine-2,2′adamantane](XXII), 2-(1-adamantanyl)pyrrolidine (XXIII), 1-(2-adamantyl)piperidine (XXIV), 3-(2-adamantyl)pyrrolidine (XXV), 2-(1-adamantyl) piperidine (XXVI), and 2-(1-adamantyl)-2-methylpyrrolidine (XXVII). See, Davies et al., Science, 144:862-863 (1964) regarding XVIII; Zoidis et al., Antimicrob. Agents Chemother., 14:153-164 (2003) regarding XIX; Kolocouris, et al., J. Med. Chem., 37:2896-2902 (1994) regarding XX and XXI; Kolocouris, et al., J. Med. Chem., 39:3307-3318 (1996) regarding XXII; Kolocouris, et al., Bioorg. Med. Chem. Lett., 9:3465-3470 (1999) regarding XXIII; Stamatiou et al., Bioorg. Med. Chem. Lett., 11:2137-2142 (2001) regarding XXIV; Stamatiou et al., Bioorg. Med. Chem. Lett., 11:5485-5492 (2003) regarding XXV and XXVI; and Zoidis et al., Bioorg. Med. Chem. Lett., 14:3341-3348 (2006) regarding XXVII.

FIG. 4 shows the structures of ribavirin (XXVIII) and viramidine (XXIX), which are inhibitors of inosine 5′-monophosphate (IMP) dehydrogenase. See Sidwell et al., Science, 177:705-706 (1972) regarding XXVIII; and Sidwell et al., Antiviral Res., 68:10-17 (2005) regarding (XXIX)

FIG. 5 shows the structures of the influenza virus RNA-polymerase inhibitors 2′-deoxy-2′-fluoroguanosine (FdG, XXX), flutimide (XXXI), thiadiazolo[2,3-a]pyrimidine (XXXII), pyrimidinyl acylthiourea (XXXIII) and T-705 (XXXIV). See, Tuttle, et al., J. Med. Chem., 36:119-125 (1993) regarding XXX; Tomassini et al., Antimicrob. Agents Chemother., 40:1189-1193 (1996) regarding XXXI; Sun et al., Bioorg. Med. Chem. Lett., 16:162-166 (2006) regarding XXXII and XXXIII; and Furuta et al., Antimicrob. Agents Chemother., 46:977-981 (2002) regarding XXXIV.

FIGS. 6A and 6B are graphs showing dry powder calcium formulations reduce influenza infection in a dose-dependent manner (A: Influenza A/WSN/33/1; B: Influenza A/Panama/2007/99). Calu3 cells exposed to no formulation were used as a control and compared to Calu3 cells exposed to dry powder formulations at different doses. The concentration of virus released by cells exposed to each aerosol formulation was quantified. Each symbol represent the mean and standard deviation of triplicate wells for each condition. Data were analyzed statistically by one way ANOVA and Tukey's multiple comparison post-test.

FIG. 7 is a graph showing a dose range of liquid formulations in a mouse influenza model that indicates that higher concentrations (4× and 8×) were most efficacious in the model. BALB/c mice (n=7-8 per group) were treated with each of the indicated concentrated liquid formulations three hours before infection with Influenza A/PR/8 (H1N1). Mice were subsequently treated three hours after infection and then BID for a total of 11 days. Animal survival, changes in animal body temperature and changes in body weight were tracked for 21 days. Mice treated with the 4× and 8× formulations exhibited increased survival compared to control mice.

FIGS. 8A and 8B are graphs showing calcium formulation treatment delays the onset of fever and reduces body temperatures in influenza infected ferrets. Body temperature changes (mean±SEM) of control ferrets, 1.3% CaCl₂-0.9% NaCl treated ferrets, 4× treated ferrets, or 8× treated ferrets. Treated ferrets exhibited (A) delayed onset of fever and had lower body temperatures over the course of the study (p<0.0001 Two-way ANOVA). (B) Treated ferrets had lower body temperatures at the time of peak fever in the control animals (36 hours post infection; *p<0.05, **p<0.01 Mann-Whitney U test; n=9 for control, n=9 for 1.3% CaCl₂-0.9% NaCl treated, and n=10 for 4× and 8× treated).

FIGS. 9A and 9B are graphs showing the administration of calcium salt formulation prevents body weight loss in influenza infected ferrets. Percent body weight loss from time zero in control ferrets, 1.3% CaCl₂,-0.9% NaCl treated ferrets, 4× treated ferrets, or 8× treated ferrets. Treated ferrets exhibited (A) less body weight loss over the course of the study (p<0.0001 Two-way ANOVA). (B) Treated ferrets had less body weight loss at the time of peak loss in the control animals (48 hours post-infection; *p<0.05, **p<0.01 Mann-Whitney U test; n=10 for all groups except 1.3% CaCl₂, 0.9% NaCl, n=9).

FIGS. 10A-C are graphs showing calcium formulations dampen the inflammatory response to influenza infection in ferrets. Nasal washes were performed once daily at the indicated times and the number of inflammatory cells in each nasal wash were enumerated. Nasal wash samples that had noticeable amounts of blood were discarded for analysis at each timepoint. The mean (±SEM) control ferrets, 1.3% CaCl₂-0.9% NaCl treated ferrets, 4× treated ferrets, or 8× treated ferrets are shown. Calcium formulation treatments reduced the number of inflammatory cells in nasal wash samples to statistically significant levels (A) over time (p<0.0001 Two way ANOVA), (B) at the peak of inflammatory cell infiltration in the controls animals (72 hours; **p<0.01 and ***p<0.001 Mann-Whitney U test; n=5 for control, n=6 for 1.3% CaCl₂, 0.9% NaCl and 4× treated, and n=7 for 8× treated at the 72 hour timepoint), and (C) at 120 hours post infection (*p<0.05 Mann-Whitney U test; n=6 for control, n=5 for 1.3% CaCl₂, 0.9% NaCl treated, n=6 for 4× treated and n=7 for 8× treated).

FIG. 11 is a bar chart showing that 1.3% CaCl₂, 0.9% NaCl, zanamivir (1.0 nM, 0.1 nM and 0.01 nM), and zanamivir (1.0 nM, 0.1 nM and 0.01 nM) in 1.3% CaCl₂-0.9% NaCl, inhibited influenza virus infection in an in vitro system. The bar chart shows that 0.1 nM or 0.01 nM zanamivir in 1.3% CaCl₂-0.9% NaCl inhibited infection to a greater extent than 1.3% CaCl₂, 0.9% NaCl or the same dose of zanamivir alone.

FIG. 12 is a bar chart showing inhibition of viral infection by 0.1 nM zanamivir, by calcium salt formulations (0.5×, 2×, 8×), and by zanamivir in the calcium salt formulations. The bar chart shows that the combination of zanamivir and each of the calcium salt formulations inhibited infection to a greater extent than the calcium salt formulations alone or zanamivir alone.

FIG. 13 is a bar chart showing inhibition of viral infection by 1.0 nM zanamivir, by calcium salt formulations (0.5×, 2×, 8×), and by zanamivir in the calcium salt formulations. The bar chart shows that the combination of zanamivir and each of the calcium salt formulations inhibited infection to a greater extent than the calcium salt formulations alone or zanamivir alone.

FIG. 14 is a bar chart showing inhibition of viral infection by dry powder formulations of zanamivir, calcium salt or zanamivir and calcium salt. The histogram shows that the formulation containing zanamivir and calcium inhibited infection to a greater extent than the calcium salt formulation or zanamivir formulation.

FIG. 15 is a bar chart showing inhibition of viral infection by 1 nM oseltamivir, by calcium salt formulations (0.5×, 2×, 8×), and by combination of oseltamivir and calcium salt formulations. The bar chart shows that the combination of oseltamivir and calcium salt formulations inhibited infection to a greater extent than the calcium salt formulations alone or oseltamivir alone.

FIG. 16 is a bar chart showing inhibition of viral infection by 10 nM ribavirin, by calcium salt formulations (0.5×, 2×, 8×), and by ribavirin in the calcium salt formulations. The bar chart shows that the combination of ribavirin and each of the calcium salt formulations did not inhibit infection to a statistically significant extent compared to the calcium salt formulations alone or ribavirin alone.

FIG. 17 is a bar chart showing inhibition of viral infection by sialidase, by 2× calcium salt formulation, and by combination of sialidase, and 2× calcium salt formulation. The bar chart shows that the combination of sialidase and the calcium salt formulation inhibited infection to a greater extent than the calcium salt formulation alone or sialidase alone.

FIG. 18 is a bar chart showing inhibition of viral infection by sialidase, by calcium salt formulations (0.5×, 2×, 8×), and by sialidase in the calcium salt formulations. The bar chart shows that the combination of sialidase and the 0.5× and 2× calcium salt formulations inhibited infection to a greater extent than the 0.5× or 2× calcium salt formulations alone or sialidase alone. The 8× calcium salt formulation in combination with sialidase did not show a statistically significant decrease in viral titer compared to the 8× calcium salt formulation alone.

FIG. 19 a graph showing rimantidine (10 nM) in combination with calcium salt formulations (0.5×, 2×, 8×) is more efficacious than rimantidine treatment alone. Cells were treated with Ca:Na formulations with and without rimantidine added to the basolateral media. The combination of 2× or 8× formulations with rimantidine produced a greater reduction in viral titer compared to rimantidine alone, however, not statistically different from the respective Ca:Na formulations. The 0.5× formulation and rimantidine significantly reduced viral titers compared to 0.5× treatment alone, however this was not statistically different from the rimantidine alone condition.

FIG. 20 is a graph showing rimantadine (1 nM) in combination with calcium salt formulations (0.5×, 2×, 8×) is more efficacious than rimantidine treatment alone. Cells were treated with Ca:Na formulations with and without rimantadine added to the basolateral media. Each of the combination treatments were more efficacious than the rimantadine treatment alone, however there was no statistical difference between any of the combination treatments with the respective Ca:Na exposure.

FIG. 21 is a graph showing calcium salt formulations (0.5×, 2×, 8×) reduce human parainfluenza virus 3 (hPIV3) in both Calu3 and normal human bronchial epithelial (NHBE) cells. Viral titers were determined in the apical washes of cells 24 hours after infection by TCID₅₀ assay using MK-2 cells. Similar to previous data obtained with influenza strains, the titer of parainfluenza was reduced in a dose responsive manner.

FIG. 22 is a graph showing calcium formulations reduce influenza infection in Calu3 cells dose responsively. Calu3 cells were treated with liquid formulations at an 8:1 molar ratio of calcium to sodium and were 0.5×, 2× and 8× in tonicity (1×=isotonic). Viral titers were determined 24 hours after treatment and the fold reduction in titer relative to an untreated control was calculated for each virus. The individual viruses tested are shown in the legend and in Table 7.

FIG. 23 is a graph showing calcium formulations reduce influenza infection in NHBE cells. NHBE cells from four different donors were treated with the indicated formulations and infected with InfluenzaA/Panama/2007/99. Viral titers were determined 24 hours after infection. Data were normalized to the untreated (air) control and presented as the log₁₀ change in TCID₅₀/mL from the control. Data are the mean±SD for 2 to 3 replicates per condition. N.D.=not determined.

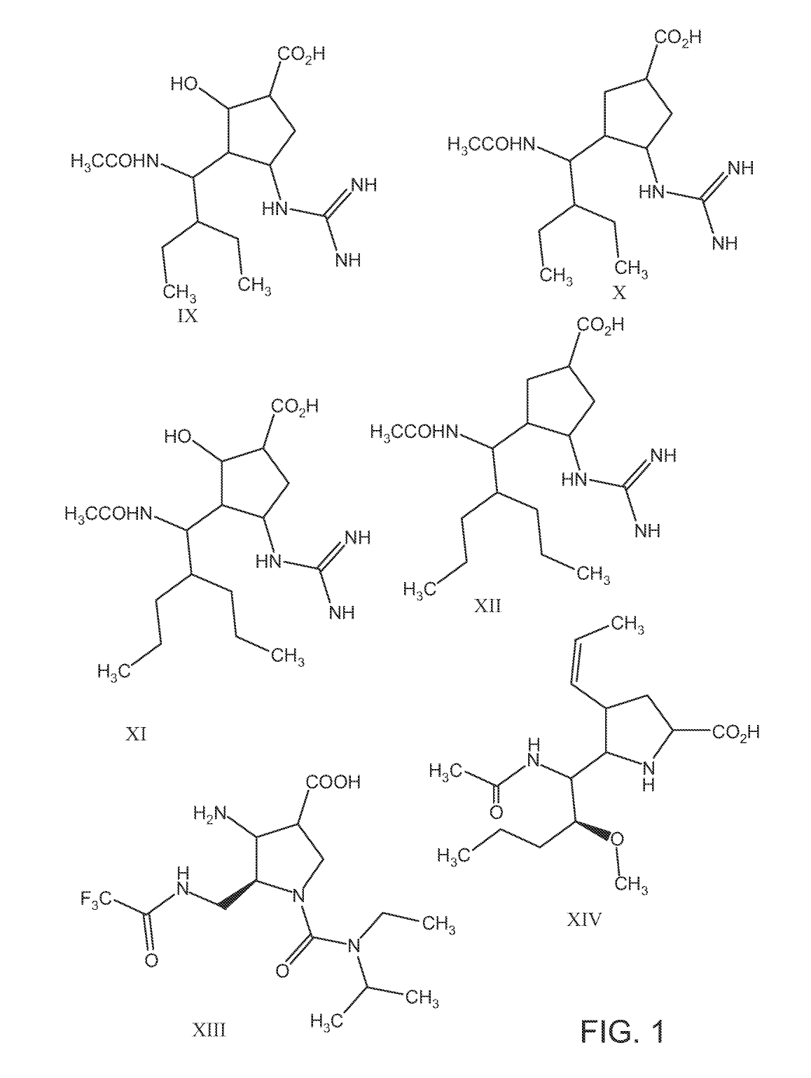

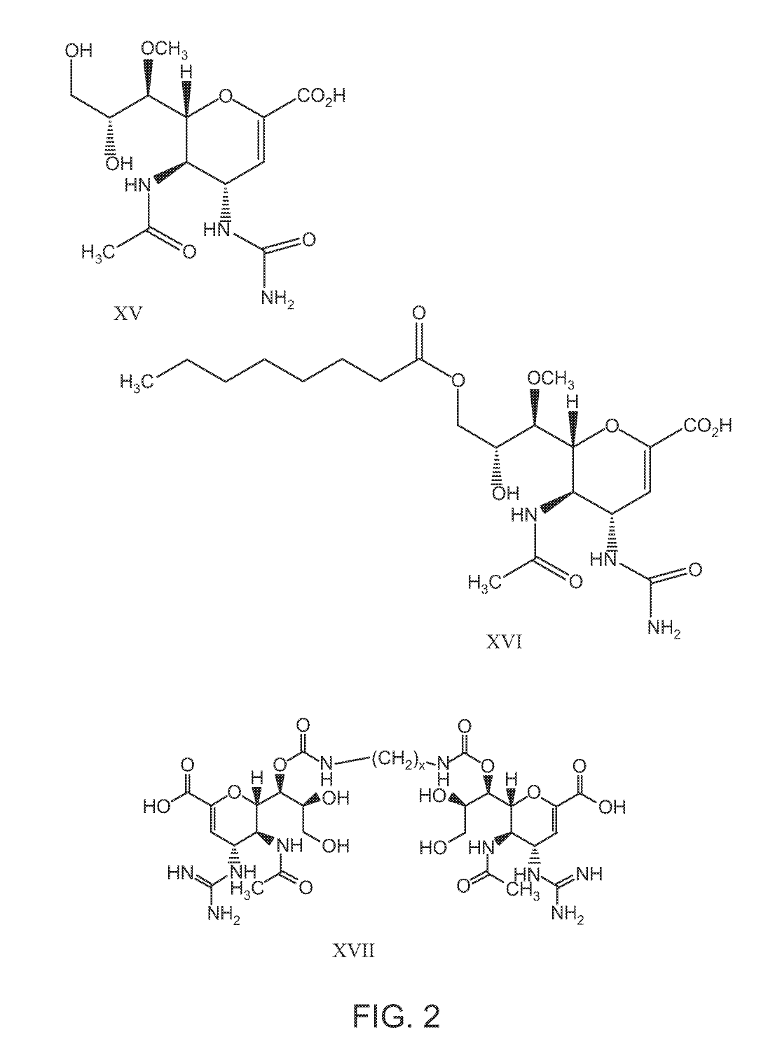

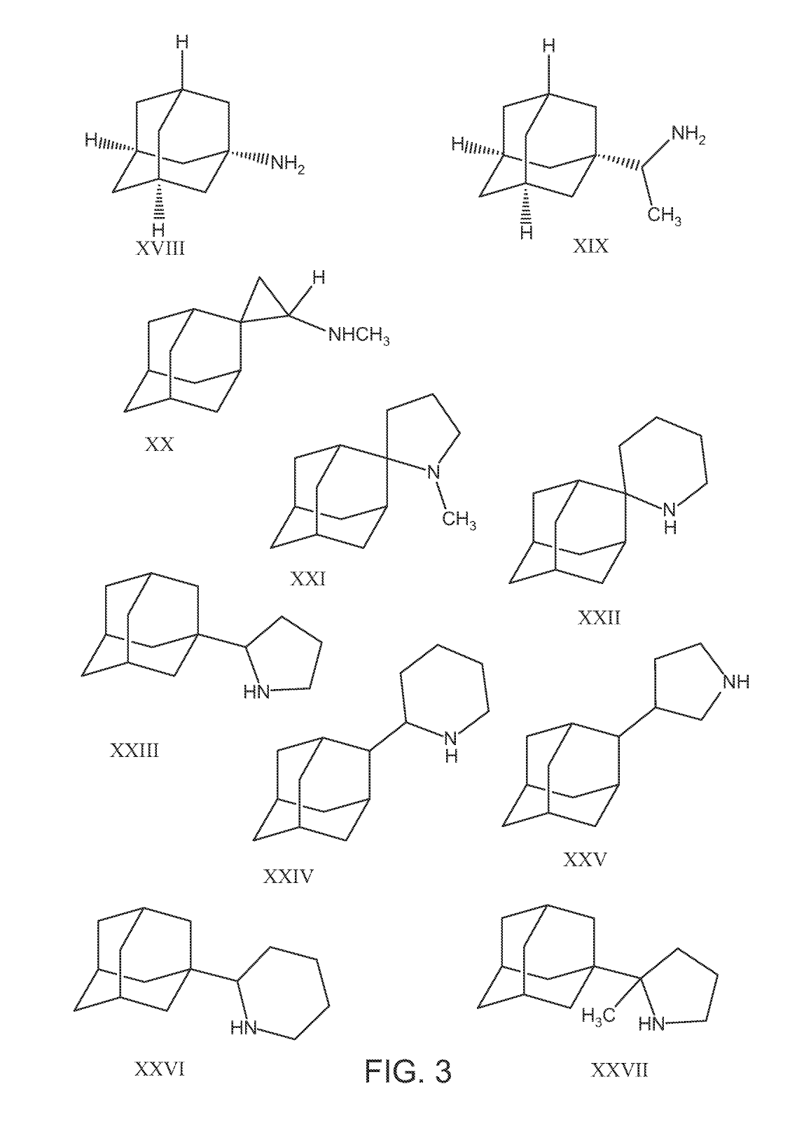

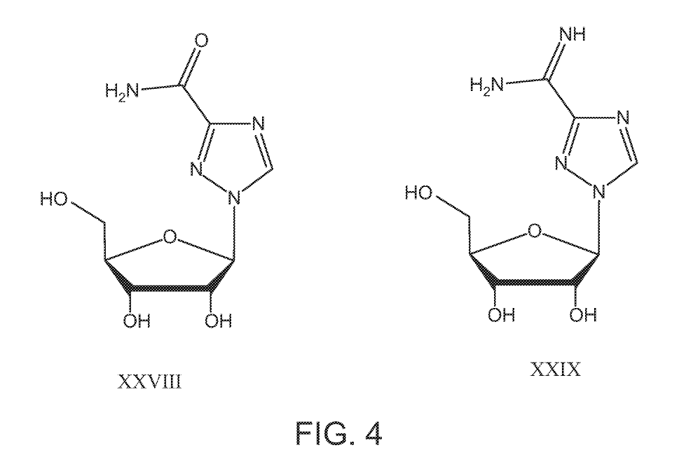

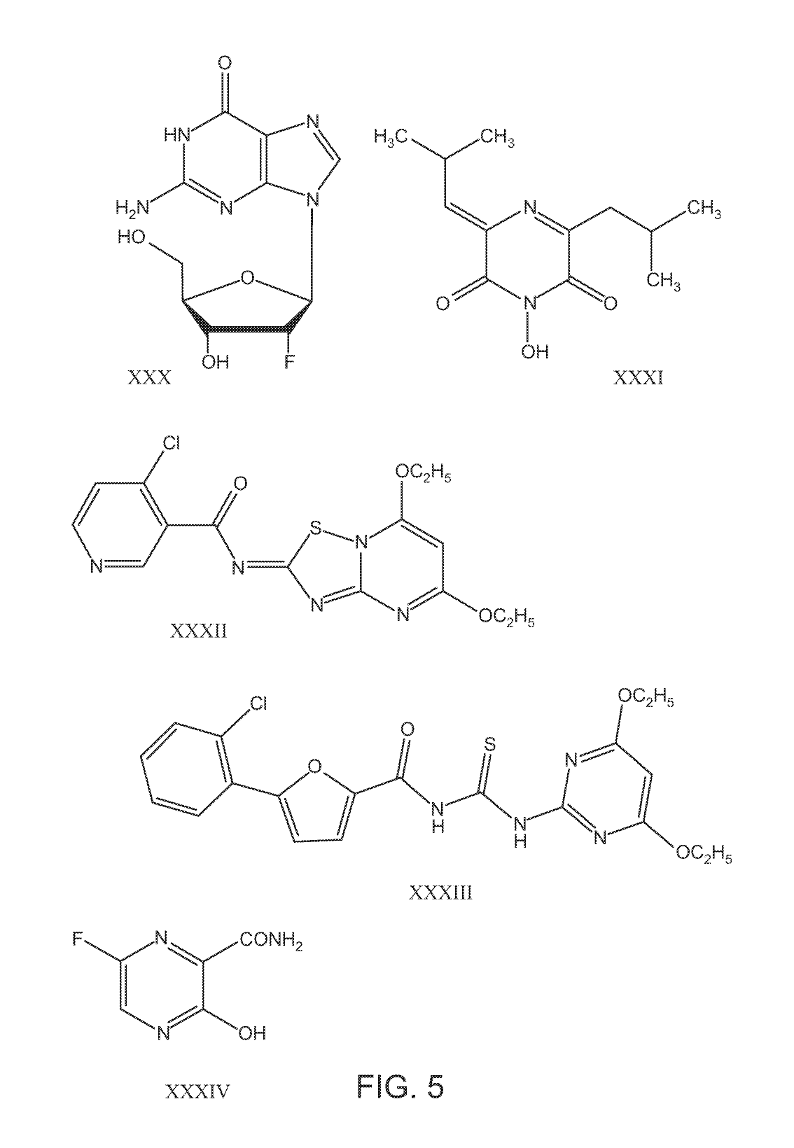

### DETAILED DESCRIPTION OF THE INVENTION

The invention relates to pharmaceutical compositions that contain a calcium salt and an anti-influenza agent, and to methods for treating or preventing influenza virus infection and influenza-like illness. As described herein, the results of studies into the inhibition of influenza virus using calcium salt formulations and anti-influenza agents, e.g. influenza virus neuraminidase inhibitors (NAIs), showed that both calcium salt formulations and anti-influenza agents inhibit the influenza virus. These studies also showed that calcium salt formulations and anti-influenza agents (e.g., NAIs) when used as combination therapy, unexpectedly resulted in greater inhibition of influenza virus replication than either the calcium salt formation or the anti-influenza agent alone. Certain doses of anti-influenza agent in combination with calcium salt formulation resulted in greater than 10-fold inhibition in comparison to the same does of anti-influenza agent in the absence of the calcium salt formulation. These results demonstrate that there is therapeutic synergy between calcium salts and anti-influenza agents, such as NAIs, that can produce superior efficacy at lower doses of anti-influenza agent. The pharmaceutical compositions provide additional benefits, for example, the formulations can be administered to treat influenza, or influenza-like illness and concomitant bacterial infections which are associated with influenza.

### DEFINITIONS

The term “salt formulation” as used herein refers to a formulation that contains an effective amount of a salt (e.g., calcium salt) as an active ingredient and is suitable for administration to the respiratory tract, e.g., by inhalation. Salt formulations do not contain any additional anti-influenza agents.

The term “pharmaceutical composition” as used herein refers to a formulation that contains an effective amount of a salt (e.g., calcium salt) as an active ingredient and an effective amount of an additional anti-influenza agent, and is suitable for administration to the respiratory tract, e.g., by inhalation.

The term “aerosol” as used herein refers to any preparation of a fine mist of particles (including liquid and non-liquid particles, e.g., dry powders), typically with a volume median geometric diameter of about 0.1 to about 30 microns or a mass median aerodynamic diameter of between about 0.5 and about 10 microns. Preferably the volume median geometric diameter for the aerosol particles is less than about 10 microns. The preferred volume median geometric diameter for aerosol particles is about 5 microns. For example, the aerosol can contain particles that have a volume median geometric diameter between about 0.1 and about 30 microns, between about 0.5 and about 20 microns, between about 0.5 and about 10 microns, between about 1.0 and about 3.0 microns, between about 1.0 and 5.0 microns, between about 1.0 and 10.0 microns, between about 5.0 and 15.0 microns. Preferably the mass median aerodynamic diameter is between about 0.5 and about 10 microns, between about 1.0 and about 3.0 microns, or between about 1.0 and 5.0 microns.

The term “synergistic effective amount” as used herein is an amount of a salt (e.g., calcium salt) and an amount of an anti-influenza agent that when administered to produce overlap in their therapeutic activities (e.g., administered substantially at the same time) produces a therapeutic or prophylactic effect that exceeds the highest single agent effect (“HSA synergy”) or exceeds Bliss independence (“Bliss synergy”). See, Boresy et al. Proc. Natl. Acad. Sci. USA 100:7977-7982 (2003) and Bliss, C. I., Ann. Appl. Biol., 26:585-615 (1939) regarding statistical analysis and synergy. For example, a synergistic effective amount can result in a therapeutic or prophylactic effect that exceeds additivism using the HSA prediction by at least about 5%, at least about 10%, at least about 15%, at least about 20%, at least about 25%, at least about 30%, at least about 35%, or at least about 40%. Alternatively or in addition, a synergistic effective amount can result in a therapeutic or prophylactic effect that exceeds additivism using the Bliss prediction by at least about 5%, at least about 10%, at least about 15%, at least about 20%, at least about 25%, at least about 30%, at least about 35%, or at least about 40%.

The term “respiratory tract” as used herein includes the upper respiratory tract (e.g., nasal passages, nasal cavity, throat, pharynx), respiratory airways (e.g., larynx, trachea, bronchi, bronchioles) and lungs (e.g., respiratory bronchioles, alveolar ducts, alveolar sacs, alveoli).

The term “dry powder” as used herein refers to a composition that contains finely dispersed respirable dry particles that are capable of being dispersed in an inhalation device and subsequently inhaled by a subject. Such dry powder or dry particle may contain up to about 15% water or other solvent, or be substantially free of water or other solvent, or be anhydrous.

As used herein, “1×” tonicity refers to a solution that is isotonic relative to normal human blood and cells. Solutions that are hypotonic or hypertonic in comparison to normal human blood and cells are described relative to a 1× solution using an appropriate multiplier. For example, a hypotonic solution may have 0.1×, 0.25× or 0.5× tonicity, and a hypertonic solution may have 2×, 3×, 4×, 5×, 6×, 7×, 8×, 9× or 10× tonicity.

The term “influenza-like illness” as used herein refers to illness that presents influenza-like symptoms, defined by the US Centers for Disease Control as fever and cough, or fever and sore throat. Influenza-like illness does not include influenza. Preferably, influenza-like illness is not RSV infection.

The invention relates to pharmaceutical compositions that contain at least one salt as an active ingredient and also contain an anti-influenza agent, and to methods for treatment, prophylaxis and for reducing contagion of influenza and influenza-like illness (e.g., parainfluenza) using the pharmaceutical compositions. The invention also relates to a method for treatment, prophylaxis and reducing contagion of influenza and influenza-like illness (e.g., parainfluenza) by administering a salt formulation to the respiratory tract, and also administering another anti-influenza agent to a subject in need thereof.

### Salt Formulations

Salt formulations for use in the invention contain at least one salt as an active ingredient, and can optionally contain additional salts or agents, and are intended for administration to the respiratory tract (e.g., by inhalation of salt formulation aerosols). Without wishing to be bound by a particular theory, it is believed that therapeutic and prophylactic benefits produced by the salt formulations and the methods described herein, result from an increase in the amount of cation (cation from the salt, such as Ca²⁺) in the respiratory tract, e.g., the lung mucus or airway lining fluid, after administration of the salt formulation.

The salt formulations can include any salt form of the elements sodium, potassium, magnesium, calcium, aluminum, silicon, scandium, titanium, vanadium, chromium, cobalt, nickel, copper, manganese, zinc, tin, silver and similar elements, that is non-toxic when administered to the respiratory tract. The salt formulation can be in any desired form, such as a solution, emulsion, suspension, or a dry powder (e.g., a dry powder). Preferred salt formulations, such as solutions and dry powders, can be aerosolized. Preferred salt formulations contain sodium salts (e.g., saline (0.15 M NaCl or 0.9% solution)), calcium salts, or mixtures of sodium salts and calcium salts. When the formulation comprises a calcium salt, a sodium salt or a combination of a calcium salt or a sodium salt, it can, if desired, also contain one or more other salts. The salt formulations can comprise multiple doses or be a unit dose composition as desired.

Suitable sodium salts include, for example, sodium chloride, sodium acetate, sodium bicarbonate, sodium carbonate, sodium sulfate, sodium stearate, sodium ascorbate, sodium benzoate, sodium biphosphate, sodium phosphate, sodium bisulfite, sodium citrate, sodium borate, sodium gluconate, sodium metasilicate, sodium lactate and the like, or a combination thereof.

Suitable calcium salts include, for example, calcium chloride, calcium carbonate, calcium acetate, calcium phosphate, calcium alginate, calcium stearate, calcium sorbate, calcium sulfate, calcium gluconate, calcium citrate, calcium lactate, and the like, or a combination thereof.

Suitable magnesium salts include, for example, magnesium carbonate, magnesium acetate, magnesium phosphate, magnesium alginate, magnesium sorbate, magnesium gluconate, magnesium citrate, magnesium lactate, magnesium sulfate, magnesium stearate, magnesium trisilicate, magnesium chloride, and the like, or a combination thereof.

Suitable potassium salts include, for example, potassium bicarbonate, potassium chloride, potassium citrate, potassium borate, potassium bisulfite, potassium biphosphate, potassium alginate, potassium benzoate, potassium lactate, potassium sulfate and the like, or a combination thereof. Additional suitable salts include cupric sulfate, chromium chloride, stannous chloride, and similar salts. Other suitable salts include zinc chloride, aluminum chloride and silver chloride.

The salt formulation is generally prepared in or comprises a physiologically acceptable carrier or excipient. For salt formulations in the form of solutions, suspensions or emulsions, any suitable carrier or excipient can be included. Suitable carriers include, for example, aqueous, alcoholic/aqueous, or alcohol solutions, emulsions or suspensions, including water, saline, ethanol/water solution, buffered media and the like. For salt formulations in the form of dry powders, suitable carrier or excipients include, for example, sugars (e.g., lactose, trehalose), sugar alcohols (e.g., mannitol, xylitol, sorbitol), amino acids (e.g., glycine, alanine, leucine, isoleucine), dipalmitoylphosphosphatidylcholine (DPPC), diphosphatidyl glycerol (DPPG), 1,2-Dipalmitoyl-sn-glycero-3-phospho-L-serine (DPPS),1,2-Dipalmitoyl-sn-glycero-3-phosphocholine (DSPC), 1,2-Distearoyl-sn-glycero-3-phosphoethanolamine (DSPE), 1-palmitoyl-2-oleoylphosphatidylcholine (POPC), fatty alcohols, polyoxyethylene-9-lauryl ether, surface active fatty, acids, sorbitan trioleate (Span 85), glycocholate, surfactin, poloxomers, sorbitan fatty acid esters, tyloxapol, phospholipids, alkylated sugars, sodium phosphate, maltodextrin, human serum albumin (e.g., recombinant human serum albumin), biodegradable polymers (e.g., PLGA), dextran, dextrin, and the like. If desired, the salt formulations can also contain additives, preservatives, or fluid, nutrient or electrolyte replenishers (See, generally, Remington's Pharmaceutical Sciences, 17th Edition, Mack Publishing Co., PA, 1985).

The salt formulation preferably contains a concentration of salt (e.g., calcium salt, sodium salt) that permits convenient administration of an effective amount of the formulation to the respiratory tract. For example, it is generally desirable that liquid formulations not be so dilute so as to require a large amount of the formulation to be nebulized in order to deliver an effective amount to the respiratory tract of a subject. Long administration periods are disfavored, and generally the formulation should be concentrated enough to permit an effective amount to be administered to the respiratory tract (e.g., by inhalation of aerosolized formulation, such as nebulized liquid or aerosolized dry powder) in no more than about 120 minutes, no more than about 90 minutes, no more than about 60 minutes, no more than about 45 minutes, no more than about 30 minutes, no more than about 25 minutes, no more than about 20 minutes, no more than about 15 minutes, no more than about 10 minutes, no more than about 7.5 minutes, no more than about 5 minutes, no more than about 4 minutes, no more than about 3 minutes, no more than about 2 minutes, no more than about 1 minute, no more than 45 seconds, or no more than about 30 seconds. For example, a liquid salt formulation (e.g., a calcium salt formulation) can contain about 0.01% to about 30% salt (w/v), between 0.1% to about 20% salt (w/v), between 0.1% to about 10% salt (w/v). Liquid formulations can contain about 0.001M to about 1.5M salt, about 0.01M to about 1.0M salt, about 0.01M to about 0.9M salt, about 0.01M to about 0.8M salt, about 0.01M to about 0.7M salt, about 0.01M to about 0.6M salt, about 0.01M to about 0.5M salt, about 0.01M to about 0.4M salt, about 0.01M to about 0.3M salt, about 0.01M to about 0.2M salt, about 0.1M to about 1.0M salt, about 0.1M to about 0.9M salt, about 0.1M to about 0.8M salt, about 0.1M to about 0.7M salt, about 0.1M to about 0.6M salt, about 0.1M to about 0.5M salt, about 0.1M to about 0.4M salt, about 0.1M to about 0.3M salt, or about 0.1M to about 0.2M salt.

Dry powder formulations can contain at least about 10% salt by weight, at least about 20% salt by weight, at least about 30% salt by weight, at least about 40% salt by weight, at least about 50% salt by weight, at least about 60% salt by weight, at least about 70% salt by weight, at least about 75% salt by weight, at least about 80% salt by weight, at least about 85% salt by weight, at least about 90% salt by weight, at least about 95% salt by weight, at least about 96% salt by weight, at least about 97% salt by weight, at least about 98% salt by weight, or at least about 99% salt by weight. For example, some dry powder formulations contain about 20% to about 80% salt by weight, about 20% to about 70% salt by weight, about 20% to about 60% salt by weight, or can consist substantially of salt(s).

Alternatively or in addition, such dry powder formulations may contain a calcium salt which provides Ca⁺² in an amount of at least about 5% Ca⁺² by weight, at least about 7% Ca⁺² by weight, at least about 10% Ca⁺² by weight, at least about 11% Ca⁺² by weight, at least about 12% Ca⁺² by weight, at least about 13% Ca⁺² by weight, at least about 14% Ca⁺² by weight, at least about 15% Ca⁺² by weight, at least about 17% Ca⁺² by weight, at least about 20% Ca⁺² by weight, at least about 25% Ca⁺² by weight, at least about 30% Ca⁺² by weight, at least about 35% Ca⁺² by weight, at least about 40% Ca⁺² by weight, at least about 45% Ca⁺² by weight, at least about 50% Ca⁺² by weight, at least about 55% Ca⁺² by weight, at least about 60% Ca⁺² by weight, at least about 65% Ca⁺² by weight or at least about 70% Ca⁺² by weight.

Alternatively or in addition, dry powder salt formulations may contain a sodium salt which provides Na⁺ in an amount of at least about 0.1% Na⁺ by weight, at least about 0.5% Na⁺ by weight, at least about 1% Na⁺ by weight, at least about 2% Na⁺ by weight, at least about 3% Na⁺ by weight, at least about 4% Na⁺ by weight, at least about 5% Na⁺ by weight, at least about 6% Na⁺ by weight, at least about 7% Na⁺ by weight, at least about 8% Na⁺ by weight, at least about 9% Na⁺ by weight, at least about 10% Na⁺ by weight, at least about 11% Na⁺ by weight, at least about 12% Na⁺ by weight, at least about 14% Na⁺ by weight, at least about 16% Na⁺ by weight, at least about 18% Na⁺ by weight, at least about 20% Na⁺ by weight, at least about 22% Na⁺ by weight, at least about 25% Na⁺ by weight, at least about 27% Na⁺ by weight, at least about 29% Na⁺ by weight, at least about 32% Na⁺ by weight, at least about 35% Na⁺ by weight, at least about 40% Na⁺ by weight, at least about 45% Na⁺ by weight, at least about 50% Na⁺ by weight, or at least about 55% Na⁺ by weight.

Preferred salt formulations contain a calcium salt. Certain calcium salts provide two or more moles of Ca²⁺ per mole of calcium salt upon dissolution. Such calcium salts may be particularly suitable to produce liquid or dry powder formulations that are dense in calcium, and therefore, can deliver an effective amount of cation (e.g., Ca²⁺, Na⁺, or Ca²⁺ and Na⁺). For example, one mole of calcium citrate provides three moles of Ca²⁺ upon dissolution. It is also generally preferred that the calcium salt is a salt with a low molecular weight and/or contains low molecular weight anion. Low molecular weight calcium salts, such as calcium salts that contain calcium ions and low molecular weight anions, are calcium dense relative to high molecular salts and calcium salts that contain high molecular weight anions. It is generally preferred that the calcium salt has a molecular weight of less than about 1000 g/mol, less than about 950 g/mol, less than about 900 g/mol, less than about 850 g/mol, less than about 800 g/mol, less than about 750 g/mol, less than about 700 g/mol, less than about 650 g/mol, less than about 600 g/mol, less than about 550 g/mol, less than about 510 g/mol, less than about 500 g/mol, less than about 450 g/mol, less than about 400 g/mol, less than about 350 g/mol, less than about 300 g/mol, less than about 250 g/mol, less than about 200 g/mol, less than about 150 g/mol, less than about 125 g/mol, or less than about 100 g/mol. In addition or alternatively, it is generally preferred that the calcium ion contributes a substantial portion of the weight to the overall weight of the calcium salt. It is generally preferred that the calcium ion weigh at least 10% of the overall calcium salt, at least 16%, at least 20%, at least 24.5%, at least 26%, at least 31%, at least 35%, or at least 38% of the overall calcium salt.

Some salt formulations contain a calcium salt in which the weight ratio of calcium to the overall weight of said calcium salt is between about 0.1 to about 0.5. For example, the weight ratio of calcium to the overall weight of said calcium salt is between about 0.15 to about 0.5, between about 0.18 to about 0.5, between about 0.2 to about 5, between about 0.25 to about 0.5, between about 0.27 to about 0.5, between about 0.3 to about 5, between about 0.35 to about 0.5, between about 0.37 to about 0.5, or between about 0.4 to about 0.5.

Some salt formulations contain a calcium salt and a sodium salt, for example 0.12 M calcium chloride in 0.15 M sodium chloride, or 1.3% (w/v) calcium chloride in 0.9% saline. Some salt formulations that contain a calcium salt and a sodium salt are characterized by the ratio of calcium:sodium (mole:mole). Suitable ratios of calcium:sodium (mole:mole) can range from about 0.1:1 to about 32:1, about 0.5:1 to about 16:1, about 1:1 to about 8:1, or about 4:1 to about 16:1. For example, the ratio of calcium:sodium (mole:mole) can be about 0.77:1, about 1:1, about 1:1.3, about 1:2, about 4:1, about 8:1 or about 16:1. In particular examples, the salt formulations contain calcium chloride and sodium chloride, and have a calcium:sodium ratio of about 8:1 (mole:mole).

In certain aspects, the salt formulation that contains a calcium salt and a sodium salt and the ratio of Ca⁺² to Na⁺ is from about 4:1 (mole:mole) to about 16:1 (mole:mole). For example, the formulations can contain a ratio of Ca⁺² to Na⁺ from about 5:1 (mole:mole) to about 16:1 (mole:mole), from about 6:1 (mole:mole) to about 16:1 (mole:mole), from about 7:1 (mole:mole) to about 16:1 (mole:mole), from about 8:1 (mole:mole) to about 16:1 (mole:mole), from about 9:1 (mole:mole) to about 16:1 (mole:mole), from about 10:1 (mole:mole) to about 16:1 (mole:mole), from about 11:1 (mole:mole) to about 16:1 (mole:mole), from about 12:1 (mole:mole) to about 16:1 (mole:mole), from about 13:1 (mole:mole) to about 16:1 (mole:mole), from about 14:1 (mole:mole) to about 16:1 (mole:mole), from about 15:1 (mole:mole) to about 16:1 (mole:mole).

In certain aspects, the salt formulation that contains a calcium salt and a sodium salt and the ratio of Ca⁺² to Na⁺ is from about 4:1 (mole:mole) to about 5:1 (mole:mole), from about 4:1 (mole:mole) to about 6:1 (mole:mole), from about 4:1 (mole:mole) to about 7:1 (mole:mole), from about 4:1 (mole:mole) to about 8:1 (mole:mole), from about 4:1 (mole:mole) to about 9:1 (mole:mole), from about 4:1 (mole:mole) to about 10:1 (mole:mole), from about 4:1 (mole:mole) to about 11:1 (mole:mole), from about 4:1 (mole:mole) to about 12:1 (mole:mole), from about 4:1 (mole:mole) to about 13:1 (mole:mole), from about 4:1 (mole:mole) to about 14:1 (mole:mole), from about 4:1 (mole:mole) to about 15:1 (mole:mole).

The salt formulations can contain a ratio of Ca⁺² to Na⁺ from about 4:1 (mole:mole) to about 12:1 (mole:mole), from about 5:1 (mole:mole) to about 11:1 (mole:mole), from about 6:1 (mole:mole) to about 10:1 (mole:mole), from about 7:1 (mole:mole) to about 9:1 (mole:mole).

In particular examples, the ratio of Ca⁺² to Na⁺ is about 4:1 (mole:mole), about 4.5:1 (mole:mole), about 5:1 (mole:mole), about 5.5:1 (mole:mole), about 6:1 (mole:mole), about 6.5:1 (mole:mole), 7:1 (mole:mole), about 7.5:1 (mole:mole), about 8:1 (mole:mole), about 8.5:1 (mole:mole), about 9:1 (mole:mole), about 9.5:1 (mole:mole), about 10:1 (mole:mole), about 10.5:1 (mole:mole), about 11:1 (mole:mole), about 11.5:1 (mole:mole), about 12:1 (mole:mole), about 12.5:1 (mole:mole), about 13:1 (mole:mole), about 13.5:1 (mole:mole), about 14:1 (mole:mole), about 14.5:1 (mole:mole), about 15:1 (mole:mole), about 15.5:1 (mole:mole), or about 16:1 (mole:mole).

In more particular examples, the ratio of Ca⁺² to Na⁺ is about 8:1 (mole:mole) or about 16:1 (mole:mole).

Aqueous liquid salt formulations of this type can vary in tonicity and in the concentrations of calcium salt and sodium salt that are present in the formulation. For example, the salt formulation can contain 0.053 M CaCl₂ and 0.007 M NaCl (0.59% CaCl₂, 0.04% NaCl) and be hypotonic, 0.106 M CaCl₂ and 0.013 M NaCl (1.18% CaCl₂, 0.08% NaCl) and be isotonic, 0.212 M CaCl₂ and 0.027 M NaCl (2.35% CaCl₂, 0.027% NaCl) and be hypertonic, 0.424 M CaCl₂ and 0.054 M NaCl (4.70% CaCl₂, 0.054% NaCl) and be hypertonic, or 0.849 M CaCl₂ and 0.106 M NaCl (9.42% CaCl₂, 0.62% NaCl) and be hypertonic.

The salt formulation can be hypotonic, isotonic or hypertonic as desired. For example, any of the salt formulations described herein may have about 0.1× tonicity, about 0.25× tonicity, about 0.5× tonicity, about 1× tonicity, about 2× tonicity, about 3× tonicity, about 4× tonicity, about 5× tonicity, about 6× tonicity, about 7× tonicity, about 8× tonicity, about 9× tonicity, about 10× tonicity, at least about 1× tonicity, at least about 2× tonicity, at least about 3× tonicity, at least about 4× tonicity, at least about 5× tonicity, at least about 6× tonicity, at least about 7× tonicity, at least about 8× tonicity, at least about 9× tonicity, at least about 10× tonicity, between about 0.1× to about 1×, between about 0.1× to about 0.5×, between about 0.5× to about 2×, between about 1× to about 4×, between about 1× to about 2×, between about 2× to about 10×, or between about 4× to about 8×.

If desired, the salt formulation can include one or more additional agents, such as mucoactive or mucolytic agents, surfactants, antibiotics, antivirals, antihistamines, cough suppressants, bronchodilators, anti-inflammatory agents, steroids, vaccines, adjuvants, expectorants, macromolecules, therapeutics that are helpful for chronic maintenance of CF.

Examples of suitable mucoactive or mucolytic agents include MUC5AC and MUC5B mucins, DNA-ase, N-acetylcysteine (NAC), cysteine, nacystelyn, dornase alfa, gelsolin, heparin, heparin sulfate, P2Y2 agonists (e.g. UTP, INS365), hypertonic saline, and mannitol.

Suitable surfactants include L-alpha-phosphatidylcholine dipalmitoyl (“DPPC”), diphosphatidyl glycerol (DPPG), 1,2-Dipalmitoyl-sn-glycero-3-phospho-L-serine (DPPS),1,2-Dipalmitoyl-sn-glycero-3-phosphocholine (DSPC), 1,2-Distearoyl-sn-glycero-3-phosphoethanolamine (DSPE),1-palmitoyl-2-oleoylphosphatidylcholine (POPC), fatty alcohols, polyoxyethylene-9-lauryl ether, surface active fatty, acids, sorbitan trioleate (Span 85), glycocholate, surfactin, poloxomers, sorbitan fatty acid esters, tyloxapol, phospholipids, and alkylated sugars.

If desired, the salt formulation can contain an antibiotic. For example, salt formulations for treating bacterial pneumonia or VAT, can further comprise an antibiotic, such as a macrolide (e.g., azithromycin, clarithromycin and erythromycin), a tetracycline (e.g., doxycycline, tigecycline), a fluoroquinolone (e.g., gemifloxacin, levofloxacin, ciprofloxacin and mocifloxacin), a cephalosporin (e.g., ceftriaxone, defotaxime, ceftazidime, cefepime), a penicillin (e.g., amoxicillin, amoxicillin with clavulanate, ampicillin, piperacillin, and ticarcillin) optionally with a β-lactamase inhibitor (e.g., sulbactam, tazobactam and clavulanic acid), such as ampicillin-sulbactam, piperacillin-tazobactam and ticarcillin with clavulanate, an aminoglycoside (e.g., amikacin, arbekacin, gentamicin, kanamycin, neomycin, netilmicin, paromomycin, rhodostreptomycin, streptomycin, tobramycin, and apramycin), a penem or carbapenem (e.g. doripenem, ertapenem, imipenem and meropenem), a monobactam (e.g., aztreonam), an oxazolidinone (e.g., linezolid), vancomycin, glycopeptide antibiotics (e.g. telavancin), tuberculosis-mycobacterium antibiotics and the like.

If desired, the salt formulation can contain an agent for treating infections with mycobacteria, such as Mycobacterium tuberculosis. Suitable agents for treating infections with mycobacteria (e.g., M. tuberculosis) include an aminoglycoside (e.g. capreomycin, kanamycin, streptomycin), a fluoroquinolone (e.g. ciprofloxacin, levofloxacin, moxifloxacin), isozianid and isozianid analogs (e.g. ethionamide), aminosalicylate, cycloserine, diarylquinoline, ethambutol, pyrazinamide, protionamide, rifampin, and the like.

If desired, the salt formulation can contain a suitable antiviral agent, such as oseltamivir, zanamavir amantidine or rimantadine, ribavirin, gancyclovir, valgancyclovir, foscavir, Cytogam® (Cytomegalovirus Immune Globulin), pleconaril, rupintrivir, palivizumab, motavizumab, cytarabine, docosanol, denotivir, cidofovir, and acyclovir. Salt formulation can contain a suitable anti-influenza agent, such as zanamivir, oseltamivir, amantadine, or rimantadine.

Suitable antihistamines include clemastine, asalastine, loratadine, fexofenadine and the like.

Suitable cough suppressants include benzonatate, benproperine, clobutinal, diphenhydramine, dextromethorphan, dibunate, fedrilate, glaucine, oxalamine, piperidione, opiods such as codine and the like.

Suitable brochodilators include short-acting beta₂ agonists, long-acting beta₂ agonists (LABA), long-acting muscarinic anagonists (LAMA), combinations of LABAs and LAMAs, methylxanthines, and the like. Suitable short-active beta₂ agonists include albuterol, epinephrine, pirbuterol, levalbuterol, metaproteronol, maxair, and the like. Suitable LABAs include salmeterol, formoterol and isomers (e.g. arformoterol), clenbuterol, tulobuterol, vilanterol (Revolair™), indacaterol, and the like. Examples of LAMAs include tiotroprium, glycopyrrolate, aclidinium, ipratropium and the like. Examples of combinations of LABAs and LAMAs include indacaterol with glycopyrrolate, indacaterol with tiotropium, and the like. Examples of methylxanthine include theophylline, and the like.

Suitable anti-inflammatory agents include leukotriene inhibitors, PDE4 inhibitors, other anti-inflammatory agents, and the like. Suitable leukotriene inhibitors include montelukast (cystinyl leukotriene inhibitors), masilukast, zafirleukast (leukotriene D4 and E4 receptor inhibitors), zileuton (5-lipoxygenase inhibitors), and the like. Suitable PDE4 inhibitors include cilomilast, roflumilast, and the like. Other anti-inflammatory agents include omalizumab (anti IgE immunoglobulin), IL-13 and IL-13 receptor inhibitors (such as AMG-317, MILR1444A, CAT-354, QAX576, IMA-638, Anrukinzumab, IMA-026, MK-6105,DOM-0910 and the like), IL-4 and IL-4 receptor inhibitors (such as Pitrakinra, AER-003,AIR-645, APG-201, DOM-0919 and the like), IL-1 inhibitors such as canakinumab, CRTh2 receptor antagonists such as AZD1981 (from AstraZeneca), neutrophil elastase inhibitor such as AZD9668 (from AstraZeneca), P38 kinase inhibitor such as losmapimed, and the like.

Suitable steroids include corticosteroids, combinations of corticosteroids and LABAs, combinations of corticosteroids and LAMAs, and the like. Suitable corticosteroids include budesonide, fluticasone, flunisolide, triamcinolone, beclomethasone, mometasone, ciclesonide, dexamethasone, and the like. Combinations of corticosteroids and LABAs include salmeterol with fluticasone, formoterol with budesonide, formoterol with fluticasone, formoterol with mometasone, indacaterol with mometasone, and the like.

Suitable expectorants include guaifenesin, guaiacolculfonate, ammonium chloride, potassium iodide, tyloxapol, antimony pentasulfide and the like.

Suitable vaccines such as nasally inhaled influenza vaccines and the like.

Suitable macromolecules include proteins and large peptides, polysaccharides and oligosaccharides, and DNA and RNA nucleic acid molecules and their analogs having therapeutic, prophylactic or diagnostic activities. Proteins can include antibodies such as monoclonal antibody. Nucleic acid molecules include genes, antisense molecules such as siRNAs that bind to complementary DNA, RNA, or ribosomes to inhibit transcription or translation.

Selected therapeutics that are helpful for chronic maintenance of CF include antibiotics/macrolide antibiotics, bronchodilators, inhaled LABAs, and agents to promote airway secretion clearance. Suitable examples of antibiotics/macrolide antibiotics include tobramycin, azithromycin, ciprofloxacin, colistin, and the like. Suitable examples of bronchodilators include inhaled short-acting beta₂ agonists such as albuterol, and the like. Suitable examples of inhaled LABAs include salmeterol, formoterol, and the like. Suitable examples of agents to promote airway secretion clearance include dornase alfa, hypertonic saline, and the like.

Dry powder formulations (e.g., dry powders) are prepared with the appropriate particle diameter, surface roughness, and tap density for localized delivery to selected regions of the respiratory tract. For example, higher density or larger particles may be used for upper airway delivery. Similarly, a mixture of different sized particles can be administered to target different regions of the lung in one administration.

As used herein, the phrase “aerodynamically light particles” refers to particles having a tap density less than about 0.4 g/cm³. The tap density of particles of a dry powder may be obtained by the standard USP tap density measurement. Tap density is a common measure of the envelope mass density. The envelope mass density of an isotropic particle is defined as the mass of the particle divided by the minimum sphere envelope volume in which it can be enclosed. Features contributing to low tap density include irregular surface texture and porous structure.

Dry powder formulations (“DPFs”) with large particle size have improved flowability characteristics, such as less aggregation (Visser, J., Dry powder Technology 58: 1-10 (1989)), easier aerosolization, and potentially less phagocytosis. Rudt, S, and R. H. Muller. J. Controlled Release, 22: 263-272 (1992); Tabata Y., and Y. Ikada. J. Biomed. Mater. Res. 22: 837-858 (1988). Dry powder aerosols for inhalation therapy are generally produced with mass median aerodynamic diameters primarily in the range of less than 5 microns, although dry powders that have any desired range in aerodynamic diameter can be produced. Ganderton D., J. Biopharmaceutical Sciences, 3:101-105 (1992); Gonda, I. “Plysico-Chemical Principles in Aerosol Delivery.” in Topics in Pharmaceutical Sciences 1991, Crommelin, D. J. and K. K. Midha, Eds., Medpharm Scientific Publishers, Stuttgart, pp. 95-115 (1992). Large “carrier” particles (containing no salt formulation) can be co-delivered with therapeutic aerosols to aid in achieving efficient aerosolization among other possible benefits. French, D. L., Edwards, D. A. and Niven, R. W., J. Aerosol Sci. 27: 769-783 (1996). Particles with degradation and release times ranging from seconds to months can be designed and fabricated by established methods in the art.

Generally, salt formulations that are dry powders may be produced by spray drying, freeze drying, jet milling, single and double emulsion solvent evaporation, and supercritical fluids. Preferably, salt formulations are produced by spray drying, which entails preparing a solution containing the salt and other components of the formulation, spraying the solution into a closed chamber, and removing the solvent with a heated gas stream.

Spray dried dry powders that contain salts with sufficient solubility in water or aqueous solvents, such as calcium chloride and calcium lactate, can be readily prepared using conventional methods. Some salts, such as calcium citrate and calcium carbonate, have low solubility in water and other aqueous solvents. Spray dried dry powders that contain such salts can be prepared using any suitable method. One suitable method involves combining other more soluble salts in solution and permitting reaction (precipitation reaction) to produce the desired salt for the dry powder formulation. For example, if a dry powder formulation comprising calcium citrate and sodium chloride is desired, a solution containing the high solubility salts calcium chloride and sodium citrate can be prepared. The precipitation reaction leading to calcium citrate is 3 CaCl₂+2 Na₃Cit→Ca₃Cit₂+6 NaCl. It is preferable that the sodium salt is fully dissolved before the calcium salt is added and that the solution is continuously stirred. The precipitation reaction can be allowed to go to completion or stopped before completion, e.g., by spray drying the solution, as desired.

Alternatively, two saturated or sub-saturated solutions are fed into a static mixer in order to obtain a saturated or supersaturated solution post-static mixing. Preferably, the post-spray drying solution is supersaturated. The two solutions may be aqueous or organic, but are preferably substantially aqueous. The post-static mixing solution is then fed into the atomizing unit of a spray dryer. In a preferable embodiment, the post-static mixing solution is immediately fed into the atomizer unit. Some examples of an atomizer unit include a two-fluid nozzle, a rotary atomizer, or a pressure nozzle. Preferably, the atomizer unit is a two-fluid nozzle. In one embodiment, the two-fluid nozzle is an internally mixing nozzle, meaning that the gas impinges on the liquid feed before exiting to the most outward orifice. In another embodiment, the two-fluid nozzle is an externally mixing nozzle, meaning that the gas impinges on the liquid feed after exiting the most outward orifice.

The resulting solution may appear clear with fully dissolved salts or a precipitate may form. Depending on reaction conditions, a precipitate may form quickly or over time. Solutions that are supersaturated or that contain a light precipitate that results in formation of a stable or metastable homogenous suspension can be spray dried.

Dry powder formulations can also be prepared by blending individual components into the final formulation. For example, a first dry powder that contains a calcium salt can be blended with a second dry powder that contains a sodium salt to produce a dry powder salt formulation that contains a calcium salt and a sodium salt. If desired, additional dry powders that contain excipients (e.g., lactose) and/or other active ingredients (e.g., antibiotic, antiviral) can be included in the blend. The blend can contain any desired relative amounts or ratios of salts, excipients and other ingredients (e.g., antibiotics, antivirals).

If desired, dry powders can be prepared using polymers, that are tailored to optimize particle characteristics including: i) interactions between the agent (e.g., salt) to be delivered and the polymer to provide stabilization of the agent and retention of activity upon delivery; ii) rate of polymer degradation and thus agent release profile; iii) surface characteristics and targeting capabilities via chemical modification; and iv) particle porosity. Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art.

Dry powder salt formulations that contain a calcium salt generally contain at least about 5% calcium salt by weight, 10% calcium salt by weight, about 15% calcium salt by weight, at least about 19.5% calcium salt by weight, at least about 20% calcium salt by weight, at least about 22% calcium salt by weight, at least about 25.5% calcium salt by weight, at least about 30% calcium salt by weight, at least about 37% calcium salt by weight, at least about 40% calcium salt by weight, at least about 48.4% calcium salt by weight, at least about 50% calcium salt by weight, at least about 60% calcium salt by weight, at least about 70% calcium salt by weight, at least about 75% calcium salt by weight, at least about 80% calcium salt by weight, at least about 85% calcium salt by weight, at least about 90% calcium salt by weight, or at least about 95% calcium salt by weight.

Alternatively or in addition, such dry powder formulations may contain a calcium salt which provides Ca⁺² in an amount of at least about 5% Ca⁺² by weight, at least about 7% Ca⁺² by weight, at least about 10% Ca⁺² by weight, at least about 11% Ca⁺² by weight, at least about 12% Ca⁺² by weight, at least about 13% Ca⁺² by weight, at least about 14% Ca⁺² by weight, at least about 15% Ca⁺² by weight, at least about 17% Ca⁺² by weight, at least about 20% Ca⁺² by weight, at least about 25% Ca⁺² by weight, at least about 30% Ca⁺² by weight, at least about 35% Ca⁺² by weight, at least about 40% Ca⁺² by weight, at least about 45% Ca⁺² by weight, at least about 50% Ca⁺² by weight, at least about 55% Ca⁺² by weight, at least about 60% Ca⁺² by weight, at least about 65% Ca⁺² by weight or at least about 70% Ca⁺² by weight.

When a dry powder salt formulation contains a calcium salt and a sodium salt the amount of sodium salt in the dry powder formulation can be dependent upon the desired calcium:sodium ratio. For example, the dry powder formulation may contain at least about 1.6% sodium salt by weight, at least about 5% sodium salt by weight, at least about 10% sodium salt by weight, at least about 13% sodium salt by weight, at least about 15% sodium salt by weight, at least about 20% sodium salt by weight, at least about 24.4% sodium salt by weight, at least about 28% sodium salt by weight, at least about 30% sodium salt by weight, at least about 30.5% sodium salt by weight, at least about 35% sodium salt by weight, at least about 40% sodium salt by weight, at least about 45% sodium salt by weight, at least about 50% sodium salt by weight, at least about 55% sodium salt by weight, or at least about 60% sodium salt by weight.

Alternatively or in addition, dry powder salt formulations may contain a sodium salt which provides Na⁺ in an amount of at least about 0.1% Na⁺ by weight, at least about 0.5% Na⁺ by weight, at least about 1% Na⁺ by weight, at least about 2% Na⁺ by weight, at least about 3% Na⁺ by weight, at least about 4% Na⁺ by weight, at least about 5% Na⁺ by weight, at least about 6% Na⁺ by weight, at least about 7% Na⁺ by weight, at least about 8% Na⁺ by weight, at least about 9% Na⁺ by weight, at least about 10% Na⁺ by weight, at least about 11% Na⁺ by weight, at least about 12% Na⁺ by weight, at least about 14% Na⁺ by weight, at least about 16% Na⁺ by weight, at least about 18% Na⁺ by weight, at least about 20% Na⁺ by weight, at least about 22% Na⁺ by weight, at least about 25% Na⁺ by weight, at least about 27% Na⁺ by weight, at least about 29% Na⁺ by weight, at least about 32% Na⁺ by weight, at least about 35% Na⁺ by weight, at least about 40% Na⁺ by weight, at least about 45% Na⁺ by weight, at least about 50% Na⁺ by weight, or at least about 55% Na⁺ by weight.

Preferred excipients for dry powder salt formulations (such as maltodextrin, mannitol or leucine) can be present in the formulations in an amount of about 50% or less (w/w). For example, a dry powder formulation may contain the amino acid leucine in an amount of about 50% or less by weight, about 45% or less by weight, about 40% or less by weight, about 35% or less by weight, about 30% or less by weight, about 25% or less by weight, about 20% or less by weight, about 18% or less by weight, about 16% or less by weight, about 15% or less by weight, about 14% or less by weight, about 13% or less by weight, about 12% or less by weight, about 11% or less by weight, about 10% or less by weight, about 9% or less by weight, about 8% or less by weight, about 7% or less by weight, about 6% or less by weight, about 5% or less by weight, about 4% or less by weight, about 3% or less by weight, about 2% or less by weight, or about 1% or less by weight. Exemplary excipients may include leucine, maltodextrin, mannitol, any combination of leucine, maltodextrin, and mannitol, or any other excipients disclosed herein or commonly used in the art.

For example, a liquid pharmaceutical formulation may contain from about 0.115 M to 1.15 M Ca²⁺ ion, from about 0.116 M to 1.15 M Ca²⁺ ion, from about 0.23 M to 1.15 M Ca²⁺ ion, from about 0.345 M to 1.15 M Ca²⁺ ion, from about 0.424 M to 1.15 M Ca²⁺ ion, from about 0.46 M to 1.15 M Ca²⁺ ion, from about 0.575 M to 1.15 M Ca²⁺ ion, from about 0.69 M to 1.15 M Ca²⁺ ion, from about 0.805 M to 1.15 M Ca²⁺ ion, from about 0.849 M to 1.15 M Ca²⁺ ion, or from about 1.035 M to 1.15 M Ca²⁺ ion. The solubility of certain calcium salts (e.g., calcium carbonate, calcium citrate) can limit the preparation of solutions. In such situations, the liquid formulation may be in the form of a suspension that contains the equivalent amount of calcium salt that would be needed to achieve the desired molar concentration.

When the salt formulation contains a sodium salt, such as a formulation that contains a calcium salt and a sodium salt, the Na⁺ ion in a liquid pharmaceutical formulation can be dependent upon the desired Ca²⁺: Na⁺ ratio. For example, the liquid formulation may contain from about 0.053 M to 0.3 M Na⁺ ion, from about 0.075 M to 0.3 M Na⁺ ion, from about 0.106 M to 0.3 M Na⁺ ion, from about 0.15 M to 0.3 M Na⁺ ion, from about 0.225 M to 0.3 M Na⁺ ion, from about 0.008 M to 0.3 M Na⁺ ion, from about 0.015 M to 0.3 M Na⁺ ion, from about 0.016 M to 0.3 M Na⁺ ion, from about 0.03 M to 0.3 M Na⁺ ion, from about 0.04 M to 0.3 M Na⁺ ion, from about 0.08 M to 0.3 M Na⁺ ion, from about 0.01875 M to 0.3 M Na⁺ ion, from about 0.0375 M to 0.3 M Na⁺ ion, from about 0.075 M to 0.6 M Na⁺ ion, from about 0.015 M to 0.6 M Na⁺ ion, or from about 0.3 M to 0.6 M Na⁺ ion.

The compositions of some preferred salt compositions are presented in Table 1. The compositions disclosed in Table 1 are non-limiting examples of salt compositions that can be administered in accordance with the methods of the invention.

TABLE 1
Liquid formulations of Calcium Chloride

| Formu- | Tonicity | | | | |
|---|---|---|---|---|---|
| lation | (1X = | CaCl₂ | CaCl₂ | NaCl | NaCl |
| # | isotonic) | (% w/v) | (M) | (% w/v) | (M) |
| 1 | 2X | 1.3 | 0.12 | 0.90 | 0.15 |
| 2 | 4X | 4.2 | 0.38 | 0.90 | 0.15 |
| 3 | 6X | 6.4 | 0.58 | 0.90 | 0.15 |
| 4 | 8X | 9.0 | 0.81 | 0.90 | 0.15 |
| 5 | 11X | 13 | 1.2 | 0.90 | 0.15 |
| 6 | 2X | 2.6 | 0.23 | n.a. | n.a. |
| 7 | 5X | 6.4 | 0.58 | n.a. | n.a. |
| 8 | 10X | 13 | 1.2 | n.a. | n.a. |
| 9 | 0.5X | 0.59 | 0.053 | 0.040 | 0.0070 |
| 10 | 1X | 1.2 | 0.11 | 0.080 | 0.013 |
| 11 | 2X | 2.4 | 0.21 | 0.16 | 0.027 |
| 12 | 4X | 4.7 | 0.42 | 0.31 | 0.053 |
| 13 | 8X | 9.4 | 0.85 | 0.62 | 0.11 |
| 14 | 0.5X | 0.32 | 0.029 | 0.23 | 0.039 |
| 15 | 1X | 0.65 | 0.058 | 0.45 | 0.077 |
| 16 | 4X | 2.6 | 0.23 | 1.8 | 0.31 |
| 17 | 8X | 5.2 | 0.47 | 3.6 | 0.62 |

Liquid formulations of Calcium Lactate

| | Tonicity | Ca- | Ca- | | |
|---|---|---|---|---|---|
| Formu- | (1X = | lactate | lactate | NaCl | NaCl |
| lation | isotonic) | (%) | (M) | (%) | (M) |
| 18 | 0.5X | 0.76 | 0.035 | 0.23 | 0.039 |
| 19 | 1X | 1.5 | 0.070 | 0.45 | 0.077 |
| 20 | 2X | 3.0 | 0.14 | 0.90 | 0.15 |
| 21 | 4X | 6.1 | 0.28 | 1.8 | 0.31 |
| 22 | 6X | 9.1 | 0.42 | 2.7 | 0.46 |
| 23 | 8X | 12 | 0.56 | 3.6 | 0.62 |
| 24 | 0.5X | 1.4 | 0.065 | 0.048 | 0.0082 |
| 25 | 1X | 2.9 | 0.13 | 0.10 | 0.016 |
| 26 | 2X | 5.7 | 0.26 | 0.19 | 0.033 |
| 27 | 3X | 9.0 | 0.41 | 0.30 | 0.052 |
| 28 | 4X | 11 | 0.52 | 0.38 | 0.065 |
| 29 | 8X | 23 | 1.0 | 0.77 | 0.13 |

Powder formulations

| | Formulation composition | | | | | |
|---|---|---|---|---|---|---|
| Formu- | | Excip- | | Calcium | | Sodium |
| lation | Excip- | ient | Calcium | salt | Sodium | salt |
| # | ient | (wt %) | salt | (wt %) | salt | (wt %) |
| 30 | Leucine | 50.0 | Calcium | 29.5 | Sodium | 20.5 |
| | | | chloride | | chloride | |
| 31 | Leucine | 50.0 | Calcium | 33.8 | Sodium | 16.2 |
| | | | acetate | | chloride | |
| 32 | Leucine | 50.0 | Calcium | 37.0 | Sodium | 13.0 |
| | | | lactate | | chloride | |
| 33 | Leucine | 50.0 | Calcium | 22.0 | Sodium | 28.0 |
| | | | chloride | | sulfate | |
| 34 | Leucine | 50.0 | Calcium | 19.5 | Sodium | 30.5 |
| | | | chloride | | citrate | |
| 35 | Leucine | 10.0 | Calcium | 66.6 | Sodium | 23.4 |
| | | | lactate | | chloride | |
| 36 | Leucine | 10.0 | Calcium | 39.6 | Sodium | 50.4 |
| | | | chloride | | sulfate | |
| 37 | Leucine | 10.0 | Calcium | 35.1 | Sodium | 54.9 |
| | | | chloride | | citrate | |
| 38 | n.a. | n.a. | Calcium | 74.0 | Sodium | 26.0 |
| | | | lactate | | chloride | |
| 39 | n.a. | n.a. | Calcium | 44.0 | Sodium | 56.0 |
| | | | chloride | | sulfate | |
| 40 | n.a. | n.a. | Calcium | 39.0 | Sodium | 61.0 |
| | | | chloride | | citrate | |
| 41 | Leucine | 10.0 | Calcium | 58.6 | Sodium | 31.4 |
| | | | lactate | | chloride | |
| 42 | Maltodextrin | 10.0 | Calcium | 58.6 | Sodium | 31.4 |
| | | | lactate | | chloride | |
| 43 | Mannitol | 10.0 | Calcium | 58.6 | Sodium | 31.4 |
| | | | lactate | | chloride | |
| 44 | Lactose | 10.0 | Calcium | 58.6 | Sodium | 31.4 |
| | | | lactate | | chloride | |
| 45 | Half leucine | 10.0 | Calcium | 58.6 | Sodium | 31.4 |
| | and half | | lactate | | chloride | |
| | maltodextrin | | | | | |
| | (wt basis) | | | | | |
| 46 | Half leucine | 20.0 | Calcium | 52.1 | Sodium | 27.9 |
| | and half | | lactate | | chloride | |
| | maltodextrin | | | | | |
| | (wt basis) | | | | | |
| 47 | Leucine | 20.0 | Calcium | 52.1 | Sodium | 27.9 |
| | | | lactate | | chloride | |
| 48 | Leucine | 12.0 | Calcium | 57.3 | Sodium | 30.7 |
| | | | lactate | | chloride | |
| 49 | Leucine | 8.0 | Calcium | 59.9 | Sodium | 32.1 |
| | | | lactate | | chloride | |

n.a. not applicable

### Pharmaceutical Compositions Containing an Anti-Influenza Agent

In one aspect, the invention is a pharmaceutical composition that comprises a salt formulation as described herein and further comprises an anti-influenza agent. The pharmaceutical compositions are intended for administration to the respiratory tract, for example by inhalation. Preferably, the salt formulation comprises a calcium salt and a sodium salt. For example, the pharmaceutical composition can comprise calcium chloride, calcium lactate or calcium citrate and also comprise sodium chloride. Particularly preferred pharmaceutical compositions contain a calcium salt and a sodium salt, wherein the ratio of calcium:sodium (mole:mole) is about 8:1, and further comprise an anti-influenza agent. Generally, the pharmaceutical compositions comprise an effective amount of salt (e.g., calcium salt) and anti-influenza agent. In some embodiments, the salt (e.g., calcium salt) and the anti-influenza agent are present in the pharmaceutical composition in a synergistic effective amount.

Many anti-influenza agents are well-known in the art, and include, for example, NAIs including long acting NAIs (LANIs), influenza M2 channel inhibitors, IMP dehydrogenase inhibitors, nucleoside analogs, influenza RNA-polymerase inhibitors, sialidase fusion proteins, sialyl multimers and polymers (e.g., sialylglycopolymers), siRNAs, oligonucleotides (e.g., phosphorothioate oligonucleotides, phosphorodiamidate morpholino oligomers), interferon alpha (e.g., PEGylated interferon alpha) and interferon inducers, such as double stranded RNA (poly(I)•poly(C)), and signal transduction inhibitors (e.g., inhibitors of Raf kinase, MEK kinase, ERK kinase, PKCalpha). (See, e.g., E. De Clercq, Nat Rev Drug Discov., 5:1015-1025 (2006)). Other anti-influenza agents can be identified by conventional screening.

Suitable examples of anti-influenza agents that can be present in a pharmaceutical composition of the invention are described herein below, as are particular embodiments of the pharmaceutical compositions. The pharmaceutical compositions of the invention can comprise any of the salt formulations and any of the anti-influenza agents described herein, for example in a liquid or dry powder formulation.

In some embodiments, the pharmaceutical composition comprises a calcium salt and an anti-influenza agent selected from the group consisting of an NAI, an M2 channel inhibitor, an IMP dehydrogenase inhibitor, an influenza RNA polymerase inhibitor, a sialidase fusion protein, a sialyl multimer or polymer, a siRNA that targets expression of influenza genes, an oligonucleotide that targets expression of influenza genes, interferon alpha, an interferon inducer, and a signal transduction inhibitor. Optionally, the pharmaceutical combination further comprises a sodium salt.

In other embodiments, the pharmaceutical composition comprises a calcium salt and an anti-influenza agent selected from the group consisting of an NAI, an influenza RNA polymerase inhibitor, a sialidase fusion protein, a sialyl multimer or polymer, a siRNA that targets expression of influenza genes, an oligonucleotide that targets expression of influenza genes, interferon alpha, an interferon inducer, and a signal transduction inhibitor. Optionally, the pharmaceutical combination further comprises a sodium salt.

In a particular embodiment, the pharmaceutical composition comprises a synergistic effective amount of a calcium salt and an anti-influenza agent selected from the group consisting of an NAI (e.g., zanamivir, laninamivir, peramivir, oseltamivir phosphate and oseltamivir carboxylate) and sialidase, wherein the composition is suitable for administration to the respiratory tract. Optionally, the pharmaceutical combination further comprises a sodium salt.

In another particular embodiment, the pharmaceutical composition comprises a synergistic effective amount of a calcium salt and an NAI (e.g., zanamivir, peramivir, oseltamivir phosphate and oseltamivir carboxylate), wherein the composition is suitable for administration to the respiratory tract. Optionally, the pharmaceutical combination further comprises a sodium salt.

In another particular embodiment, the pharmaceutical composition comprises a synergistic effective amount of a calcium salt and sialidase, wherein the composition is suitable for administration to the respiratory tract. Optionally, the pharmaceutical combination further comprises a sodium salt.

### NAIs

NAIs inhibit the influenza virus neuraminidase and inhibit release of new virions from infected cells, thereby inhibiting the infection of new host cells and spread of infection. Suitable NAIs can be identified using any suitable method. Several suitable methods are well known in the art. For example, the neuraminadase assay disclosed in Yamashita et al., Antimicrob. Agents Chemothera., 53:186-192 (2009) at page 187 can be used to identify an NAI.

NAIs suitable for use in the invention include compounds of formula (I) or formula (Ia):

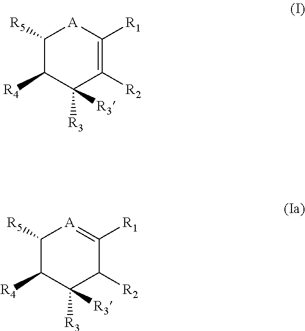

wherein, in formula (I), A is oxygen, carbon or sulfur, and in formula (Ia), A is nitrogen or carbon;

R₁ is COOH, P(O)(OH)₂, NO₂,SOOH, SO₃H, tetrazol, CH₂CHO, CHO or CH(CHO)₂,

R₂ is H, OR₆, F, Cl, Br, CN, NHR₆, SR₆ or CH₂×, wherein X is NHR₆, halogen or OR₆;

R₆ is hydrogen; an acyl group having 1 to 4 carbon atoms; a linear or cyclic alkyl group having 1 to 6 carbon atoms, or a halogen-substituted analogue thereof; an allyl group or an unsubstituted aryl group or an aryl substituted by a halogen, an OH group, an NO₂ group, an NH₂ group or a COOH group;

R₃ and R_{3′}, are the same or different, and each denotes hydrogen, CN, NHR₆, N₃, 5R₆, ═N—OR₆, OR₆, guanidino, N(R₆)₂

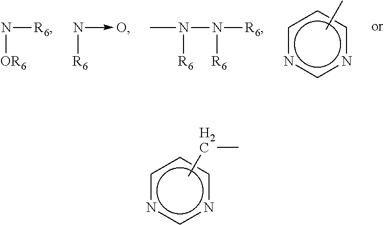

R₄ is NHR₆, SR₆, OR₆, COOR₆, NO₂, C(R₆)₃, CH₂COOR₆, CH₂NO₂ or CH₂NHR₆;

R₅ is CH₂YR₆, CHYR₆CH₂YR₆, CHYR₆CHYR₆CH₂CN, or CHYR₆CHYR₆CH₂YR₆, wherein Y is O, S, NH or H, and successive Y moieties in an R₅ group are the same or different;

and salts (e.g., physiologically or pharmaceutically acceptable salts) or derivatives thereof,

provided that in formula (I)
(i) when R₃ or R_{3′} is OR₆ or hydrogen, and A is oxygen or sulphur, then the compound cannot have both
   (a) an R₂ that is hydrogen and
   (b) an R₄ that is O-acyl or NH-acyl, and
(ii) R₆ represents a covalent bond when Y is hydrogen, and

provided that in formula (Ia),
(i) when R₃ or R_{3′}, is OR₆ or hydrogen, and A is nitrogen, then the compound cannot have both
   (a) an R₂ that is hydrogen, and
   (b) an R₄ that is NH-acyl; and
(ii) R₆ represents a covalent bond when Y is hydrogen.

Preferred NAIs of formulas I or Ia have the formula

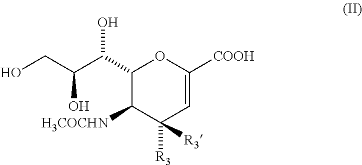

wherein R₃ is hydrogen or R_{3′};

R_{3′} is —N3, —CN, —CH₂NH₂ or —NR₈R₉; and

R₈ and R₉ are independently hydrogen, a linear or cyclic alkyl group of 1 to 6 carbon atoms, an acyl group or substituted acyl group of 1 to 6 carbon atoms, —C(NH)NH₂, —CH₂COOH, —CH₂CH₂—OH or —CH₂CH(R₁₀)R₁₁,

R₁₀ and R₁₁ are independently oxygen or ═NR₁₂, and

R₁₂ is hydrogen, —OH, —OCH₃, —NH₂ or —N(CH₃)₂. Suitable methods for producing compounds of formulas I, Ia and II are disclosed in U.S. Pat. No. 5,648,379, the substituents for compounds of formulas I, Ia and II are further described on columns 2-5 of U.S. Pat. No. 5,648,379.

More preferred NAIs are of formula II wherein R3 is —NH—C(═NH)—NH2. A particularly preferred NAI is 5-(acetylamino)-4-[(aminoiminomethyl)-amino]-2,6-anhydro-3,4,5-trideoxy-D-glycero-D-galacto-non-2-enoic acid (zanamivir, formula III). A suitable method for producing the compound of formula III is disclosed as Example 3 on column 16 of U.S. Pat. No. 5,360,817.

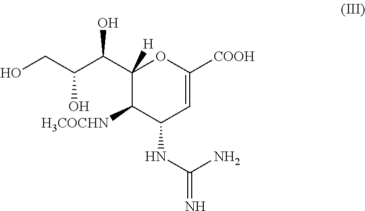

NAIs suitable for use in the invention also include compounds of formula IV or V:

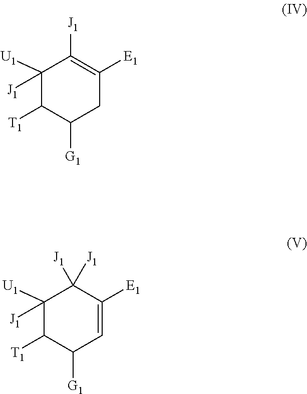

E₁ is —(CR₁₃R₁₃)ₘW₁; G₁ is N3, —CN, —OH, —OR₁₈ₐ, —NO₂, or —(CR₁₃R₁₃)ₘW₂;

T₁ is —NR₁₃W₃, a heterocycle, or is taken together with U₁ or G₁ to form a group having the structure

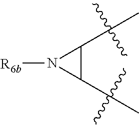

U₁ is H or —X₁W₆;

each J₁ is independently H, F or Cl;

R₁₃ is H or alkyl of 1 to 6 carbon atoms;

R₁₄ is R₁₅ or R₁₆ wherein each R₁₆ is independently substituted with 0 to 3 R₁₅ groups;

R₁₅ is F, Cl, Br, I, —CN, N₃, —OR₁₈ₐ, —OR₁₃, —N(R₁₃)₂, —N(R₁₃)(R18b), —N(R_{18b})₂, —SR₁₃, —SR₁₈ₐ, —C(O)OR₁₃, —C(O)OR₁₈ₐ, —OC(O)R₁₃, —NR₁₃C(O)R₁₃, —N(R_{18b})C(O)R₁₃, —C(O)N(R₁₃)₂, —C(O)N(R_{18b})(R₁₃), —C(O)N(R_{18b})₂, —C(NR₁₃)(N(R₁₃)₂), —C(N(R_{18b}))(N(R_{18b})₂), .═O, .═S, ═N(R_{18b}) or ═N(R₁₃);

R₁₆ is alkyl of 1 to 6 carbon atoms, alkenyl of 2 to 6 carbon atoms, or alkynyl of 2 to 6 carbon atoms;

R₁₇ is R₁₆ wherein each R₁₆ is substituted with 0 to 3 R₁₅ groups;

R₁₈ₐ is H or a protecting group for hydroxyl or thio;

R_{18b} is H or a protecting group for amino;

W₁ is a group comprising an acidic hydrogen or an R₁₈ₐ-protected acidic group;

W₂ is a group comprising a basic heteroatom or an R_{18b}-protected basic heteroatom;

W₃ is W₄ or W₅;

W₄ is R₁₇ or —C(O)R₁₇, —C(O)W₅, —SO₂R₁₇, or —SO₂W₅;

W₅ is carbocycle or heterocycle wherein each W₅ is independently substituted with 0 to 3 R₁₄ groups;

W₆ is R₁₃, W₅, —C(O)OR₁₈ₐ, —C(O)NR_{18b}R_{18b}, —C(NR_{18b})NR_{18b}R_{18b}, —C(S)NR_{18b}R_{18b}, —C(O)R₁₃, —CHR₁₃W₇, —CH(R₁₃)ₐW₇ or —C(O)W₇, where a is 0 or 1, but is 0 when W₇ is divalent;

W₇ is R₁₅ or an alkyl of 1 to 4 carbons substituted with 1 to 3 R₁₅ groups;

X₁ is a bond, —CR₁₃R₁₃—, —(CR₁₃R₁₃)₂—, —O—, —NR₁₃—, —N(OR₁₃)—, —N(NR₁₃R₁₃)—, —S—, —SO—, or —SO₂—; and

each m1 is independently an integer from 0 to 2; with the proviso that when:

(a) E₁ is —CO₂H, —P(O)(OH)₂, —NO₂, —SO₂H, —SO₃H, tetrazolyl, —CH₂CHO, —CHO, or —CH(CHO)₂;

(b) G₁ is —CN, —NHR₂₀, —OR₂₀, guanidino, —N(R₂₀)(OR₂₀), —N(H)(R₂₀)N(R₂₀)₂, unsubstituted pyrimidinyl, or unsubstituted (pyrimidinyl)methyl;

(c) T₁ is —NHR₂₀, —SR₂₀, —OR₂₀, —CO₂R₂₀, —NO₂, —C(R₂₀)₃, —CH₂CO₂R₂₀, —CH₂NO₂, or —CH₂NHR₂₀; and R₂₀ is H; an acyl group having 1 to 4 carbon atoms; a linear or cyclic alkyl group having 1 to 6 carbon atoms, or a halogen-substituted analogue thereof; an allyl group or an unsubstituted aryl group or an aryl substituted by a halogen, an OH group, an NO₂ group, an NH₂ group or a COOH group;

(d) each J₁ is H; and

(e) X₁ is a bond, —CH₂— or —CH₂CH₂—;

then W₆ is not H, W₇ or —CH₂W₇, wherein W₇ is H, —OR₁₈ₐ, —OR₁₃, —N(R₁₃)₂, —N(R₁₃)(R_{18b}), —N(R_{18b})₂, —SR₁₃, or —SR₁₈ₐ; and salts (e.g., physiologically or pharmaceutically acceptable salts) or derivatives thereof.

Some preferred NAIs have the formula

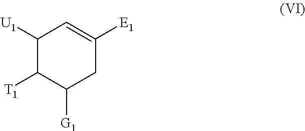

E₁ is —CO₂H, —CO₂R₁₇, —CO₂R₁₇ₐW₅ or —CO₂W₅;

G₁ is —N(R₁₉)₂, —N(R₁₉)C(N(R₁₉))(N(R₁₉)₂), or —C(R₁₉)₂—N(R₁₉)₂;

T₁ is —NH(C(O)CH₃), —NH(C(O)CH₂F), —NH(C(O)CHF₂), or —NH(C(O)CF₃);

U₁ is —OR₁₆, —SR₁₆, NHR₁₆ or N(R₁₆)₂;

R₁₇ₐ is independently alkylene of 1 to 12 carbon atoms, alkenylene of 2 to 12 carbon atoms, or alkynylene of 2-12 carbon atoms any one of which alkylene, alkenylene or alkynylene is substituted with 0-3 R₁₅ groups;

R₁₉ is independently H or R₁₇;

R₁₅, R₁₆, R₁₇, and W₅ are as defined for formulas IV and V, and the salts, solvates, resolved enantiomers and purified diastereomers thereof. Suitable methods for producing compounds of formulas IV, V and VI are disclosed in U.S. Pat. No. 5,866,601, the substituents for compounds of formulas IV, V and VI are further described on columns 3-22 of U.S. Pat. No. 5,866,601.

Particularly preferred NAI are (3R,4R,5S)-4-acetylamino-5-amino-3(1-ethylpropoxy)-1-cyclohexene-1-carboxylic acid ethyl ester (oseltamivir, formula VII) and (3R,4R,5S)-4-acetylamino-5-amino-3(1-ethylpropoxy)-1-cyclohexene-1-carboxylic acid and salts thereof (oseltamivir carboxylate, formula VIII). Suitable methods for producing the compounds of formula VII and VIII are disclosed as scheme 34 on column 71 and scheme 28 on column 67 of U.S. Pat. No. 5,763,483.

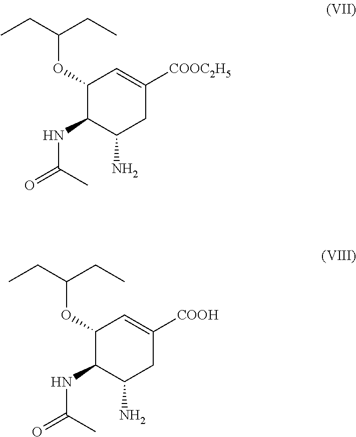

Other NAIs suitable for use in the invention include the compounds shown in Formulas IX-XVI, and salts (e.g., physiologically or pharmaceutically acceptable salts) or derivatives thereof.

If desired, the pharmaceutical composition can comprise a dimeric NAI. Suitable methods for preparing dimeric compounds, such as NAIs, are conventional and well-known in the art. For example, Macdonald et al., Antimicrob. Agents Cheother., 48:4542-4549 (2004) discloses a series of dimers of zanamivir (XVII) and suitable methods for their production at page 4545. In some embodiments, the pharmaceutical composition comprises an NAI of formula XVII, wherein x is about 4 to about 14 (e.g., X is 4, 5, 6, 7, 8, 9, 10, 11, 12, 13 or 14) and salts (e.g., physiologically or pharmaceutically acceptable salts) or derivatives thereof.

In some embodiments, the pharmaceutical composition comprises a salt formulation, as described herein, and an NAI, such as an NAI of any one of formulas I-XVII. For example, the pharmaceutical composition can comprise an NAI of formula I or Ia, an NAI of formula II, an NAI of formula III, an NAI of formula IV or V, an NAI of formula VI, an NAI or formula VII or VIII, an NAI of any one of formulas IX-XIV(preferably of formula IX), an NAI of formula XV or XVI, or an NAI of formula XVII. Preferably the salt formulation is a calcium salt formulation, such as a calcium chloride, calcium lactate or calcium citrate formulation. More preferably, the salt formulation comprises a calcium salt and a sodium salt. For example, the pharmaceutical composition can comprise calcium chloride, calcium lactate or calcium citrate, and also comprise sodium chloride. In particular embodiments, the pharmaceutical composition contains a calcium salt and a sodium salt, wherein the ratio of calcium:sodium (mole:mole) is about 8:1, and further comprises an NAI, such as an NAI of any one of formulas I-XVII.

In particular embodiments, the pharmaceutical composition comprises a calcium salt (e.g., calcium chloride, calcium lactate, calcium citrate), a sodium salt (e.g., sodium chloride) and the NAI of formula III (zanamivir). The ratio of calcium:sodium (mole:mole) can be about 8:1.

In other particular embodiments, the pharmaceutical composition comprises a calcium salt (e.g., calcium chloride, calcium lactate, calcium citrate), sodium chloride and the NAI of formula VII (oseltamivir). The ratio of calcium:sodium (mole:mole) can be about 8:1.

In other particular embodiments, the pharmaceutical composition comprises a calcium salt (e.g., calcium chloride, calcium lactate, calcium citrate), a sodium salt (e.g., sodium chloride) and the NAI of formula VIII (oseltamivir carboxylate). The ratio of calcium:sodium (mole:mole) can be about 8:1.

In other particular embodiments, the pharmaceutical composition comprises a calcium salt (e.g., calcium chloride, calcium lactate, calcium citrate), a sodium salt (e.g., sodium chloride) and the NAI of formula IX (peramivir). The ratio of calcium:sodium (mole:mole) can be about 8:1.

In other particular embodiments, the pharmaceutical composition comprises a calcium salt (e.g., calcium chloride, calcium lactate, calcium citrate), a sodium salt (e.g., sodium chloride) and the NAI of formula XVI (laninamivir). The ratio of calcium:sodium (mole:mole) can be about 8:1.

Generally, the pharmaceutical compositions comprise an effective amount of salt (e.g., calcium salt) and of NAI. In some embodiments, the salt (e.g., calcium salt) and the NAI are present in the pharmaceutical composition in a synergistic effective amount.

### M2 Channel Inhibitors

M2 channel inhibitors inhibit the influenza virus M2 (matrix 2) channel and block the transport of protons into the interior of the virus particle within host cell endosomes, and thereby inhibit viral uncoating. Suitable M2 channel inhibitors can be identified using any suitable method. Several suitable methods are well known in the art, such as the assay disclosed in Giffin et al., FEBS Letters 357:269-274 (1995).

M2 channel inhibitors suitable for use in the invention include compounds of formulas XVII-XXVII and salts (e.g., physiologically or pharmaceutically acceptable salts) or derivatives thereof.

In some embodiments, the pharmaceutical composition comprises a salt formulation, as described herein, and an M2 channel inhibitor, such as a compound of any one of formulas XVIII-XXVII. Preferably the salt formulation is a calcium salt formulation, such as a calcium chloride, calcium lactate or calcium citrate formulation. More preferably, the salt formulation comprises a calcium salt and a sodium salt. For example, the pharmaceutical composition can comprise, calcium chloride, calcium lactate or calcium citrate and also comprise a sodium salt (e.g., sodium chloride). In particular embodiments, the pharmaceutical composition contains a calcium salt and a sodium salt, wherein the ratio calcium:sodium (mole:mole) is about 8:1, and further comprises an M2 channel inhibitor, such as an M2 channel inhibitor of any one of formulas XVIII-XXVII. Generally, the pharmaceutical compositions comprise an effective amount of salt (e.g., calcium salt) and of an M2 channel inhibitor. In some embodiments, the salt (e.g., calcium salt) and the M2 channel inhibitor are present in the pharmaceutical composition in a synergistic effective amount.

### IMP Dehydrogenase Inhibitors

IMP dehydrogenase inhibitors inhibit the cellular enzyme IMP dehydrogenase which is important for the biosynthesis of viral RNA, and thereby inhibit viral replication. Suitable IMP dehydrogenase inhibitors can be identified using any suitable method. Several suitable methods are well known in the art, such as the assays disclosed in Hatakeyama et al., J. Biol. Chem., 267:20734-20739 (1992); and in Example 6 of U.S. Pat. No. 5,358,855. Several inhibitors of IMP dehydrogenase are known in the art, such as tiazofurin, mycophenolic acid, 2-amino-1,3,4-thiadiazole, VX-497 (see, Markland et al., Antimicrob. Agents Chemother., 44:859-866 (2000)). IMP dehydrogenase inhibitors suitable for use in the invention include the forgoing compounds, and the compounds of formulas XXVIII and XXIX and salts (e.g., physiologically or pharmaceutically acceptable salts) or derivatives thereof.

In some embodiments, the pharmaceutical composition comprises a salt formulation, as described herein, and an IMP dehydrogenase inhibitor, such as tiazofurin, mycophenolic acid, 2-amino-1,3,4-thiadiazole, VX-497, or a compound of formula XXVIII or XXIX. Preferably the salt formulation is a calcium salt formulation, such as a calcium chloride, calcium lactate or calcium citrate formulation. More preferably, the salt formulation comprises a calcium salt and a sodium salt. For example, the pharmaceutical composition can comprise, calcium chloride, calcium lactate or calcium citrate and also comprise a sodium salt (e.g., sodium chloride). In particular embodiments, the pharmaceutical composition contains a calcium salt and a sodium salt, wherein the ratio of calcium:sodium (mole:mole) is about 8:1, and further comprises an IMP dehydrogenase inhibitor, such as tiazofurin, mycophenolic acid, 2-amino-1,3,4-thiadiazole, VX-497, or a compound of formula XXVIII or XXIX.

In another particular embodiment, the pharmaceutical composition comprises a calcium salt (e.g., calcium chloride, calcium lactate, calcium citrate, calcium sulfate), a sodium salt (e.g., sodium chloride) and the IMP dehydrogenase inhibitor of formula XXVIII (ribavirin) or formula XXIX (viramidine).

Generally, the pharmaceutical compositions comprise an effective amount of salt (e.g., calcium salt) and of an IMP dehydrogenase inhibitor. In some embodiments, the salt (e.g., calcium salt) and the IMP dehydrogenase inhibitor are present in the pharmaceutical composition in a synergistic effective amount.

### Influenza RNA Polymerase Inhibitors

Influenza RNA polymerase inhibitors inhibit the RNA polymerase (RNA replicase) of the virus and inhibit transcription and replication of the viral RNA. Influenza RNA polymerase inhibitors can be identified using any suitable method. Several suitable methods are well known in the art, such as the influenza virus transcription assay disclosed in Tomassine et al., Antimocrob. Agents Chemother. 40:1189-1193 (1996) at page 1190.

Influenza RNA polymerase inhibitors suitable for use in the invention include compounds of formulas XXX-XXXIV and salts (e.g., physiologically or pharmaceutically acceptable salts) or derivatives thereof.

In some embodiments, the pharmaceutical composition comprises a salt formulation, as described herein, and an influenza RNA polymerase inhibitor, such as any one of the compounds of formulas XXX-XXXIV. Preferably the salt formulation is a calcium salt formulation, such as a calcium chloride, calcium lactate, calcium sulfate or calcium citrate formulation. More preferably, the salt formulation comprises a calcium salt and a sodium salt. For example, the pharmaceutical composition can comprise calcium chloride, calcium lactate, calcium sulfate or calcium citrate and also comprise a sodium salt (e.g., sodium chloride). In particular embodiments, the pharmaceutical composition contains a calcium salt and a sodium salt, wherein the ratio of calcium:sodium (mole:mole) is about 8:1, and further comprises an influenza RNA polymerase inhibitor, such as any one of the compounds of formulas XXX-XXXIV.

In another particular embodiment, the pharmaceutical composition comprises a calcium salt (e.g., calcium chloride, calcium lactate, calcium citrate, calcium sulfate), a sodium salt (e.g., sodium chloride) and the influenza RNA polymerase inhibitor of formula XXX (2′-deoxy-2′-fluoroguanosine), formula XXXI (flutimide), or formula XXXIV (T-705).

Generally, the pharmaceutical compositions comprise an effective amount of salt (e.g., calcium salt) and of an influenza RNA polymerase inhibitor. In some embodiments, the salt (e.g., calcium salt) and the influenza RNA polymerase inhibitor are present in the pharmaceutical composition in a synergistic effective amount.

### Sialidase and Sialidase Fusion Proteins

Sialidase fusion proteins that contain a sialidase catalytic domain and a portion that anchors or targets the fusion protein to respiratory epithelium can be administered by inhalation to remove terminal sialic acid residues from glycoproteins on the respiratory epithelium, thereby inhibiting infection by influenza virus. The fusion protein can contain the catalytic domain from any suitable sialidase. For example, the fusion protein can contain the catalytic domain from the known sialidase of Arthrobacter ureafociens, Clostridium perfringes, Vibrio cholerae or Actinomyces viscosus. The portion that anchors or targets the fusion protein to respiratory epithelium can be any protein or portion of a protein that binds to respiratory epithelium, such as a protein that binds glycosaminoglycans on respiratory epithelium. Suitable portions that anchor or target the fusion protein to respiratory epithelium include heparin binding domains. Many proteins are known to contain heparin binding domains, such as antithrombin, L-selectin, P-selectin, fibronectin, amphiregulin and the like. Fusion proteins can be readily prepared by a person of skill in the art using conventional recombinant DNA methodologies, or other suitable methods. A preferred sialidase fusion protein contains the catalytic domain of the sialidase of A. viscosus and the heparin-binding sequence of human amphiregulin. The preparation of one such sialidase fusion protein, DAS181, which contains amino acid residues 125-145 of amphiregulin (GenBank entry AAH09799) fused via its amino terminus to the catalytic domain of A. viscous sialidase (amino acids 274-667 in GenBank entry X62276), is described in Malakhov et al., Antimicrob. Agents Chemother., 50:1470-1479 (2006) at pages 1471-1474.

In some embodiments, the pharmaceutical composition comprises a salt formulation, as described herein, and a sialidase fusion protein as described herein, such as DAS181. Preferably the salt formulation is a calcium salt formulation, such as a calcium chloride, calcium lactate, calcium sulfate or calcium citrate formulation. More preferably, the salt formulation comprises a calcium salt and a sodium salt. For example, the pharmaceutical composition can comprise calcium chloride, calcium lactate, calcium sulfate or calcium citrate and also comprise a sodium salt (e.g., sodium chloride). In particular embodiments, the pharmaceutical composition contains a calcium salt and a sodium salt, wherein the ratio of calcium:sodium (mole:mole) is about 8:1, and further comprises sialidase fusion protein, such as DAS181.

Generally, the pharmaceutical compositions comprise an effective amount of salt (e.g., calcium salt) and of sialidase fusion protein. In some embodiments, the salt (e.g., calcium salt) and the sialidase fusion protein are present in the pharmaceutical composition in a synergistic effective amount.

### Sialic Acid Containing Multimers and Polymers

Sialic acid containing multimers and polymers can inhibit influenza virus attachment to cells by binding to the virus surface protein hemagglutinin (HA), which is the receptor for host cell sialooligosaccharides. Sialic acid containing multimers that inhibit HA binding to host cells can be identified using any suitable method, such as assays that assess whether a compound inhibits influenza virus-induced agglutination of red blood cells, such as the assay described in Reuter et al. Biochong. Chem. 10:271-278 (1999). Suitable agents include oligomers and polymers, including dendromers, that contain sialyl groups (e.g., 6′-sialyl(N-acetylylactosamine), Neu5Acα2-6Galβ1-4GlcNAc, Neu5Ac3αF and the like; See Gambaryan et al, Antiviral Research 55:201-205 (2002) and Chao-Tan et al., Glycobiology, 12:183-190 (2002)). Suitable sialic acid containing multimers and polymers include, for example, 6′SLN-PAA, disclosed at page 117 of Gambaryan et al, Antiviral Research 55:201-205 (2002), and Neu5Ac3αF-DSP disclosed at page 184 of Chao-Tan et al., Glycobiology, 12:183-190 (2002).

In some embodiments, the pharmaceutical composition comprises a salt formulation, as described herein, and a sialic acid containing multimer or polymer as described herein, such as 6′SLN-PAA or Neu5Ac3αF-DSP. Preferably the salt formulation is a calcium salt formulation, such as a calcium chloride, calcium lactate, calcium sulfate or calcium citrate formulation. More preferably, the salt formulation comprises a calcium salt and a sodium salt. For example, the pharmaceutical composition can comprise, calcium chloride, calcium lactate, calcium sulfate or calcium citrate and also comprise a sodium salt (e.g., sodium chloride). In particular embodiments, the pharmaceutical composition contains a calcium salt and a sodium salt, wherein the ratio of calcium:sodium (mole:mole) is about 8:1, and further comprises a sialic acid containing multimer or polymer, such as 6′SLN-PAA or Neu5Ac3αF-DSP.

In other particular embodiments, the pharmaceutical composition comprises a calcium salt (e.g., calcium chloride, calcium lactate, calcium citrate, calcium sulfate), a sodium salt (e.g., sodium chloride) and 6′SLN-PAA or Neu5Ac3αF-DSP.

Generally, the pharmaceutical compositions comprise an effective amount of salt (e.g., calcium salt) and of sialic acid containing multimer or polymer. In some embodiments, the salt (e.g., calcium salt) and the sialic acid containing multimer or polymer are present in the pharmaceutical composition in a synergistic effective amount.

siRNA and Oligonucleotides

Small interfering RNA (siRNA), double stranded RNA 20-25 nucleotides in length, can direct sequence specific degradation of mRNA or viral RNA to interfere with gene expression and/or replication. Antisense oligonucleotides that form duplexes with target mRNA sequences can also inhibit gene expression, although through a different mechanism. To be effective to inhibit influenza virus infection, siRNA and antisense oligonucleotides must be delivered into the epithelial cells of the respiratory tract. Several suitable methods for delivery of these types of agents into the epithelial cells of the respiratory tract are well-known in the art, for example, by fast intravenous injection of a large volume of solution containing siRNA or oligonucleotides (hydrodynamic transfection) and various in vivo transfection techniques, such as by administering complexes containing polyethyleneimine or other cationic polymers and siRNA or oligonucleotides directly to the lung or intravenously. (See, e.g., Ge et al., Proc. Natl. Acad. Sci. USA, 101:8676-8681 (2004).) If desired, oligonucleotides that are nuclease resistant can be used, such as oligos that contain phosphorothioate linkages (S-oligos) or a phosphorodiamidate morpholino backbone (PMO).

Some oligonucleotides that can be used in the invention are not antisense oligonucleotides, but have direct anti-influenza activity. Suitable examples of such oligonucleotides include the non-sequence complementary oligonucleotides disclosed in U.S. Pat. No. 7,358,068, which discloses, inter alia, phosphorothioated oligonucleotides that contain the sequence (c)₄₀ and (ac)₂₀ (See, SEQ ID NOS:22 and 24 of U.S. Pat. No. 7,358,068, respectively).

Suitable siRNAs and oligonucleotides include those that target the influenza virus Hemagglutinin (HA), neuraminidase (NA), nucleoprotein (NP), matrix channel 1 (MP1), matrix channel 2 (MP2), nonstructural gene 1 (NS1), nonstructural gene 2 (NS2), polymerase subunit A (PA), polymerase subunit B1 (PB1), polymerase subunit B2 (PB2), or gene PB1-F2. Particular siRNAs that are suitable include (NP)-1496, sense 5′-GGAUCUUAUUUCUUCGGAGdTdT-3′, antisense 5′-dTdTCCUAGAAUAAAGAAGCCUC-3′; and (PA)-2087, sense 5′-GCAAUUGAGGAGUGCCUGAdTdT-3′, antisense 5′-dTdTCGUUAACUCCUCACGGACU-3′.

In some embodiments, the pharmaceutical composition comprises a salt formulation, as described herein, and an siRNA or oligonucleotide as described herein, such as an siRNA or oligonucleotide that targets expression of Hemagglutinin

(HA), neuraminidase (NA), nucleoprotein (NP), matrix channel 1 (MP1), matrix channel 2 (MP2), nonstructural gene 1 (NS1), nonstructural gene 2 (NS2), polymerase subunit A (PA), polymerase subunit B1 (PB1), polymerase subunit B2 (PB2), or gene PB1-F2. Preferably the salt formulation is a calcium salt formulation, such as a calcium chloride, calcium lactate, calcium sulfate or calcium citrate formulation. More preferably, the salt formulation comprises a calcium salt and a sodium salt. For example, the pharmaceutical composition can comprise calcium chloride, calcium lactate, calcium sulfate or calcium citrate and also comprise a sodium salt (e.g., sodium chloride). In particular embodiments, the pharmaceutical composition contains a calcium salt and a sodium salt, wherein the ratio of calcium:sodium (mole:mole) is about 8:1, and further comprises a siRNA or oligonucleotide as described herein, such as an siRNA or oligonucleotides that targets expression of Hemagglutinin (HA), neuraminidase (NA), nucleoprotein (NP), matrix channel 1 (MP1), matrix channel 2 (MP2), nonstructural gene 1 (NS1), nonstructural gene 2 (NS2), polymerase subunit A (PA), polymerase subunit B1 (PB1), polymerase subunit B2 (PB2), or gene PB1-F2. In particular embodiments, the pharmaceutical composition contains a calcium salt and a sodium salt, wherein the ratio of calcium:sodium (mole:mole) is about 8:1, and further comprises oligonucleotides that targets a non-coding sequence of the influenza virus and may direct the degradation of the viral genome itself.

In other particular embodiments, the pharmaceutical composition comprises a calcium salt (e.g., calcium chloride, calcium lactate, calcium citrate, calcium sulfate), a sodium salt (e.g., sodium chloride) and an siRNA selected from the group consisting of (NP)-1496 and (PA)-2087.

Generally, the pharmaceutical compositions comprise an effective amount of salt (e.g., calcium salt) and of a siRNA or oligonucleotide. In some embodiments, the salt (e.g., calcium salt) and the siRNA or oligonucleotide are present in the pharmaceutical composition in a synergistic effective amount.

In some embodiments, the pharmaceutical composition comprises a salt formulation, as described herein, and interferon or an interferon inducer as described herein, such as double stranded RNA (poly(I)•poly(C)). Preferably the salt formulation is a calcium salt formulation, such as a calcium chloride, calcium lactate, calcium sulfate or calcium citrate formulation. More preferably, the salt formulation comprises a calcium salt and a sodium salt. For example, the pharmaceutical composition can comprise calcium chloride, calcium lactate, calcium sulfate or calcium citrate and also comprise a sodium salt (e.g., sodium chloride). In particular embodiments, the pharmaceutical composition contains a calcium salt and a sodium salt, wherein the ratio of calcium:sodium (mole:mole) is about 8:1, and further comprises an interferon or interferon inducer as described herein, such as double stranded RNA (poly(I)•poly(C)).

Generally, the pharmaceutical compositions comprise an effective amount of salt (e.g., calcium salt) and of an interferon or interferon inducer. In some embodiments, the salt (e.g., calcium salt) and the interferon or interferon inducer are present in the pharmaceutical composition in a synergistic effective amount.

In some embodiments, the pharmaceutical composition comprises a salt formulation, as described herein, and a signal transduction inhibitor described herein, such as an inhibitor of Raf kinase, MEK kinase, ERK kinase, PKCalpha. Preferably the salt formulation is a calcium salt formulation, such as a calcium chloride, calcium lactate, calcium sulfate or calcium citrate formulation. More preferably, the salt formulation comprises a calcium salt and a sodium salt. For example, the pharmaceutical composition can comprise calcium chloride, calcium lactate, calcium sulfate or calcium citrate and also comprise a sodium salt (e.g., sodium chloride). In particular embodiments, the pharmaceutical composition contains a calcium salt and a sodium salt, wherein the ratio of calcium:sodium (mole:mole) is about 8:1, and further comprises a signal transduction inhibitor described herein, such as an inhibitor of Raf kinase, MEK kinase, ERK kinase, PKCalpha.

Generally, the pharmaceutical compositions comprise an effective amount of salt (e.g., calcium salt) and of a signal transduction inhibitor. In some embodiments, the salt (e.g., calcium salt) and the signal transduction inhibitor are present in the pharmaceutical composition in a synergistic effective amount.

### Administering Salt Formulations and Pharmaceutical Compositions

In another aspect the invention relates to methods for treatment, prophylaxis and for reducing contagion of influenza. The methods comprise administering an effective amount of a salt formulation or pharmaceutical composition to the respiratory tract of an individual suspected of having influenza, with confirmed influenza or at risk for influenza (e.g., at risk for infection by influenza virus). The methods also comprise administering an effective amount of a salt formulation or pharmaceutical composition to the respiratory tract of an individual with influenza-like illness (e.g., parainfluenza). Advantageously, when a pharmaceutical composition of the invention is administered to an individual, the individual receives the beneficial effect of the anti-influenza agent and the beneficial effect of the salt formulation, which has its own therapeutic benefits and also can potentiate and synergize with the anti-influenza agent to produce superior therapy.

The salt formulations and pharmaceutical compositions are intended for administration to the respiratory tract (e.g., to the mucosal surface of the respiratory tract), and can be administered in any suitable form, such as a solution, a suspension, a spray, a mist, a foam, a gel, a vapor, droplets, particles, or a dry powder form. Preferably the salt formulation is aerosolized for administration to the respiratory tract. Salt formulations can be aerosolized for administration via the oral airways using any suitable method and/or device, and many suitable methods and devices are conventional and well-known in the art. For example, salt formulations can be aerosolized using a metered dose inhaler (e.g., a pressurized metered dose inhaler (pMDI) including HFA propellant, or a non-HFA propellant) with or without a spacer or holding chamber, a nebulizer, an atomizer, a continuous sprayer, an oral spray or a dry powder inhaler (DPI). Salt formulations can be aerosolized for administration via the nasal airways using a nasal pump or sprayer, a metered dose inhaler (e.g., a pressurized metered dose inhaler (pMDI) including HFA propellant, or a non-HFA propellant) with or without a spacer or holding chamber, a nebulizer with or without a nasal adapter or prongs, an atomizer, a continuous sprayer, or a dry powder inhaler (DPI). Salt formulations can also be delivered to the nasal mucosal surface via, for example, nasal wash and to the oral mucosal surfaces via, for example, an oral wash. Salt formulations can be delivered to the mucosal surfaces of the sinuses via, for example, nebulizers with nasal adapters and nasal nebulizers with oscillating or pulsatile airflows.

The geometry of the airways is an important consideration when selecting a suitable method for producing and delivering aerosols of salt formulations and pharmaceutical compositions to the lungs. The lungs are designed to entrap particles of foreign matter that are breathed in, such as dust. There are three basic mechanisms of deposition: impaction, sedimentation, and Brownian motion (J. M. Padfield. 1987. In: D. Ganderton & T. Jones eds. Drug Delivery to the Respiratory Tract, Ellis Harwood, Chicherster, U.K.). Impaction occurs when particles are unable to stay within the air stream, particularly at airway branches. Impacted particles are adsorbed onto the mucus layer covering bronchial walls and eventually cleared from the lungs by mucociliary action. Impaction mostly occurs with particles over 5 μm in diameter. Smaller particles (those less than about 3 μm in diameter) tend to stay within the air stream and to be transported deep into the lungs by sedimentation. Sedimentation often occurs in the lower respiratory system where airflow is slower. Very small particles (those less than about 0.6 μm) can deposit by Brownian motion.

Influenza virus typically replicates initially in the upper airways and later in the lung epithelia. Therefore, the salt formulations and pharmaceutical compositions can be delivered to the upper respiratory airway and/or the lung (e.g., deep lung). Delivery to the upper respiratory airways is advantageous for prophylaxis or to prevent early infection from spreading.

For administration, a suitable method (e.g., nebulization, dry powder inhaler) is selected to produce aerosols with the appropriate particle size for preferential delivery to the desired region of the respiratory tract, such as the deep lung (generally particles between about 0.6 microns and 5 microns in diameter), the upper airway (generally particles of about 3 microns or larger diameter), or the deep lung and the upper airway.

An “effective amount” of salt formulation, pharmaceutical compositions and/or anti-influenza agent is administered to an individual in need thereof, such as an individual who has influenza, has an influenza-like illness (e.g., parainfluenza), is experiencing influenza-like symptoms or who is at risk for infection by influenza virus. An effective amount is an amount that is sufficient to achieve the desired therapeutic or prophylactic effect, such as an amount sufficient to reduce influenza, influenza-like illness (e.g., parainfluenza) or influenza-like symptoms, to reduce duration of illness, to reduce influenza virus titer in an individual, to reduce parainfluenza virus titer in an individual, to inhibit influenza virus passing through the lung mucus or airway lining fluid, to inhibit parainfluenza virus passing through the lung mucus or airway lining fluid, to reduce the number of days that infected individuals experience flu-like symptoms, to decrease the incidence or rate of influenza virus infection and/or to increase mucociliary clearance (Groth et al, Thorax, 43(5):360-365 (1988)). Because the salt formulations and pharmaceutical compositions are administered to the lungs, generally by inhalation, the dose that is administered is related to the composition of the salt formulation (e.g., calcium salt concentration), the rate and efficiency of aerosolization (e.g., nebulization rate and efficiency), and the time of exposure (e.g., nebulization time). For example, substantially equivalent doses can be administered using a concentrated liquid salt formulation and a short (e.g., 5 minutes) nebulization time, or using a dilute liquid salt formulation and a long (e.g., 30 minutes or more) nebulization time, or using a dry powder formulation and a dry powder inhaler. The clinician of ordinary skill can determine appropriate dosage of salt (cation) and anti-influenza agent based on these considerations and other factors, for example, the individual's age, sensitivity, tolerance and overall well-being. The salt formulations can be administered in a single dose or multiple doses as indicated.

In some aspects, the invention is a therapeutic method that comprises administering to an individual suspected of having influenza or at risk of having influenza an effective amount of a pharmaceutical composition of the invention. For example, in some embodiments the individual is suspected of having influenza and has one or more symptoms of influenza. Symptoms of influenza are well-known and include fever and cough, or fever and sore throat. Additional symptoms of influenza include headache, tiredness, runny or stuffy nose, body aches, diarrhea and vomiting.

In some embodiments, the method is for treating influenza infection, and comprises administering to an individual in need thereof an effective amount of a pharmaceutical composition of the invention. In other embodiments, the method is for the prophylaxis of influenza infection and comprises administering to an individual at risk for infection by influenza virus an effective amount of a pharmaceutical composition of the invention. In other embodiments, the method is for reducing the spread of influenza infection comprising administering to an individual infected by influenza virus or at risk for infection by influenza virus an effective amount of a pharmaceutical composition of the invention.

In the methods of the invention, it is generally preferred that the pharmaceutical composition is administered by inhalation, for example, as an aerosol.

As described herein, it is believed that the therapeutic and prophylactic effects of the salt formulations are the result of an increased amount of cation (the cation of the salt, such as Ca²⁺) in the lung following administration of a salt formulation. It is also believed that the increased amount of cation in the respiratory tract (e.g., lung) augments and synergizes with anti-influenza agents, such as zanamivir. Accordingly, since the amount of cation provided can vary depending upon the particular salt selected, dosing can be based on the desired amount of cation to be delivered to the lung. For example, one mole of calcium chloride (CaCl₂) dissociates to provide one mole of Ca²⁺, but one mole of tricalcium phosphate (Ca₃(PO₄)₂) can provide three moles of Ca²⁺.

Generally, an effective amount of a pharmaceutical formulation will deliver a dose of about 0.001 mg Ca⁺²/kg body weight/dose to about 2 mg Ca⁺²/kg body weight/dose, about 0.002 mg Ca⁺²/kg body weight/dose to about 2 mg Ca⁺²/kg body weight/dose, about 0.005 mg Ca⁺²/kg body weight/dose to about 2 mg Ca⁺²/kg body weight/dose, about 0.01 mg Ca⁺²/kg body weight/dose to about 2 mg Ca⁺²/kg body weight/dose, about 0.01 mg Ca⁺²/kg body weight/dose to about 60 mg Ca⁺²/kg body weight/dose, about 0.01 mg Ca⁺²/kg body weight/dose to about 50 mg Ca⁺²/kg body weight/dose, about 0.01 mg Ca⁺²/kg body weight/dose to about 40 mg Ca⁺²/kg body weight/dose, about 0.01 mg Ca⁺²/kg body weight/dose to about 30 mg Ca⁺²/kg body weight/dose, about 0.01 mg Ca⁺²/kg body weight/dose to about 20 mg Ca⁺²/kg body weight/dose, about 0.01 mg Ca⁺²/kg body weight/dose to about 10 mg Ca⁺²/kg body weight/dose, about 0.01 mg Ca⁺²/kg body weight/dose to about 5 mg Ca⁺²/kg body weight/dose, about 0.01 mg Ca⁺²/kg body weight/dose to about 2 mg Ca⁺² cation/kg body weight/dose, about 0.02 mg Ca⁺²/kg body weight/dose to about 2 mg Ca⁺²/kg body weight/dose, about 0.03 mg Ca⁺²/kg body weight/dose to about 2 mg Ca⁺²/kg body weight/dose, about 0.04 mg Ca⁺²/kg body weight/dose to about 2 mg Ca⁺²/kg body weight/dose, about 0.05 mg Ca⁺²/kg body weight/dose to about 2 mg Ca⁺²/kg body weight/dose, about 0.1 mg Ca⁺²/kg body weight/dose to about 2 mg Ca⁺²/kg body weight/dose, or about 0.1 mg Ca⁺²/kg body weight/dose to about 1 mg Ca⁺²/kg body weight/dose, or about 0.1 mg Ca⁺²/kg body weight/dose to about 0.5 mg Ca⁺²/kg body weight/dose, or about 0.2 mg Ca⁺²/kg body weight/dose to about 0.5 mg Ca⁺²/kg body weight/dose, or about 0.18 mg Ca⁺²/kg body weight/dose, about 0.001 mg Ca⁺²/kg body weight/dose, about 0.005 mg Ca⁺²/kg body weight/dose, about 0.01 mg Ca⁺²/kg body weight/dose, about 0.02 mg Ca⁺²/kg body weight/dose, or about 0.5 mg Ca⁺²/kg body weight/dose.

In some embodiments, a salt formulation that comprises a calcium salt (e.g., calcium chloride, calcium lactate, calcium citrate) is administered in an amount sufficient to deliver a dose of about 0.1 mg Ca²⁺/kg body weight/dose to about 2 mg Ca²⁺/kg body weight/dose, or about 0.1 mg Ca²⁺/kg body weight/dose to about 1 mg Ca²⁺/kg body weight/dose, or about 0.1 mg Ca²⁺/kg body weight/dose to about 0.5 mg Ca²⁺/kg body weight/dose, or about 0.18 mg Ca²⁺/kg body weight/dose.

In some embodiments the amount of calcium delivered to the respiratory tract (e.g., lungs, respiratory airway) is about 0.001 mg Ca⁺²/kg body weight/dose to about 2 mg Ca⁺²/kg body weight/dose, about 0.002 mg Ca⁺²/kg body weight/dose to about 2 mg Ca⁺²/kg body weight/dose, about 0.005 mg Ca⁺²/kg body weight/dose to about 2 mg Ca⁺²/kg body weight/dose, about 0.01 mg Ca⁺²/kg body weight/dose to about 2 mg Ca⁺²/kg body weight/dose, about 0.01 mg Ca⁺²/kg body weight/dose to about 60 mg Ca⁺²/kg body weight/dose, about 0.01 mg Ca⁺²/kg body weight/dose to about 50 mg Ca⁺²/kg body weight/dose, about 0.01 mg Ca⁺²/kg body weight/dose to about 40 mg Ca⁺²/kg body weight/dose, about 0.01 mg Ca⁺²/kg body weight/dose to about 30 mg Ca⁺²/kg body weight/dose, about 0.01 mg Ca⁺²/kg body weight/dose to about 20 mg Ca⁺²/kg body weight/dose, about 0.01 mg Ca⁺²/kg body weight/dose to about 10 mg Ca⁺²/kg body weight/dose, about 0.01 mg Ca⁺²/kg body weight/dose to about 5 mg Ca⁺²/kg body weight/dose, about 0.01 mg Ca⁺²/kg body weight/dose to about 2 mg Ca⁺²/kg body weight/dose, about 0.02 mg Ca⁺²/kg body weight/dose to about 2 mg Ca⁺²/kg body weight/dose, about 0.03 mg Ca⁺²/kg body weight/dose to about 2 mg Ca⁺²/kg body weight/dose, about 0.04 mg Ca⁺²/kg body weight/dose to about 2 mg Ca⁺²/kg body weight/dose, about 0.05 mg Ca⁺²/kg body weight/dose to about 2 mg Ca⁺²/kg body weight/dose, about 0.1 mg Ca⁺²/kg body weight/dose to about 2 mg Ca⁺²/kg body weight/dose, about 0.1 mg Ca⁺²/kg body weight/dose to about 1 mg Ca⁺²/kg body weight/dose, about 0.1 mg Ca⁺²/kg body weight/dose to about 0.5 mg Ca⁺²/kg body weight/dose, about 0.2 mg Ca⁺²/kg body weight/dose to about 0.5 mg Ca⁺²/kg body weight/dose, about 0.18 mg Ca⁺²/kg body weight/dose, about 0.001 mg Ca⁺²/kg body weight/dose, about 0.005 mg Ca⁺²/kg body weight/dose, about 0.01 mg Ca⁺²/kg body weight/dose, about 0.02 mg Ca⁺²/kg body weight/dose, or about 0.5 mg Ca⁺²/kg body weight/dose about 0.01 mg/kg body weight to about 60 mg/kg body weight/dose, or about 0.01 mg/kg body weight/dose to about 50 mg/kg body weight/dose, about 0.01 mg/kg body weight/dose to about 40 mg/kg body weight/dose, about 0.01 mg/kg body weight/dose to about 30 mg/kg body weight/dose, about 0.01 mg/kg body weight/dose to about 20 mg/kg body weight/dose, 0.01 mg/kg body weight/dose to about 10 mg/kg body weight/dose, about 0.1 mg/kg body weight/dose to about 10 mg/kg body weight/dose, or about 1 mg/kg body weight/dose to about 10 mg/kg body weight/dose, or about 0.01 mg/kg body weight/dose to about 1 mg/kg body weight/dose, or about 0.1 mg/kg body weight/dose to about 1 mg/kg body weight/dose, or about 0.2 mg/kg body weight/dose to about 0.5 mg/kg body weight/dose.

In other embodiments the amount of calcium delivered to the upper respiratory tract (e.g., nasal cavity) is of about 0.001 mg Ca⁺²/kg body weight/dose to about 2 mg Ca⁺²/kg body weight/dose, about 0.002 mg Ca⁺²/kg body weight/dose to about 2 mg Ca⁺²/kg body weight/dose, about 0.005 mg Ca⁺²/kg body weight/dose to about 2 mg Ca⁺²/kg body weight/dose, about 0.01 mg Ca⁺²/kg body weight/dose to about 2 mg Ca⁺²/kg body weight/dose, about 0.01 mg Ca⁺²/kg body weight/dose to about 60 mg Ca⁺²/kg body weight/dose, about 0.01 mg Ca⁺²/kg body weight/dose to about 50 mg Ca⁺²/kg body weight/dose, about 0.01 mg Ca⁺²/kg body weight/dose to about 40 mg Ca⁺²/kg body weight/dose, about 0.01 mg Ca⁺²/kg body weight/dose to about 30 mg Ca⁺²/kg body weight/dose, about 0.01 mg Ca⁺²/kg body weight/dose to about 20 mg Ca⁺²/kg body weight/dose, about 0.01 mg Ca⁺²/kg body weight/dose to about 10 mg Ca⁺²/kg body weight/dose, about 0.01 mg Ca⁺²/kg body weight/dose to about 5 mg Ca⁺²/kg body weight/dose, about 0.01 mg Ca⁺²/kg body weight/dose to about 2 mg Ca⁺²/kg body weight/dose, about 0.02 mg Ca⁺²/kg body weight/dose to about 2 mg Ca⁺²/kg body weight/dose, about 0.03 mg Ca⁺²/kg body weight/dose to about 2 mg Ca⁺²/kg body weight/dose, about 0.04 mg Ca⁺²/kg body weight/dose to about 2 mg Ca⁺²/kg body weight/dose, about 0.05 mg Ca⁺²/kg body weight/dose to about 2 mg Ca⁺²/kg body weight/dose, about 0.1 mg Ca⁺²/kg body weight/dose to about 2 mg Ca⁺²/kg body weight/dose, about 0.1 mg Ca⁺²/kg body weight/dose to about 1 mg Ca⁺²/kg body weight/dose, about 0.1 mg Ca⁺²/kg body weight/dose to about 0.5 mg Ca⁺²/kg body weight/dose, about 0.2 mg Ca⁺²/kg body weight/dose to about 0.5 mg Ca⁺²/kg body weight/dose, about 0.18 mg Ca⁺²/kg body weight/dose, about 0.001 mg Ca⁺²/kg body weight/dose, about 0.005 mg Ca⁺²/kg body weight/dose, about 0.01 mg Ca⁺²/kg body weight/dose, about 0.02 mg Ca⁺²/kg body weight/dose, or about 0.5 mg Ca⁺²/kg body weight/dose about 0.01 mg/kg body weight/dose to about 60 mg/kg body weight/dose, or about 0.01 mg/kg body weight/dose to about 50 mg/kg body weight/dose, about 0.01 mg/kg body weight/dose to about 40 mg/kg body weight/dose, about 0.01 mg/kg body weight/dose to about 30 mg/kg body weight/dose, about 0.01 mg/kg body weight/dose to about 20 mg/kg body weight/dose, 0.01 mg/kg body weight/dose to about 10 mg/kg body weight/dose, about 0.1 mg/kg body weight/dose to about 10 mg/kg body weight/dose, or about 1 mg/kg body weight/dose to about 10 mg/kg body weight/dose, or about 0.01 mg/kg body weight/dose to about 1 mg/kg body weight/dose, or about 0.1 mg/kg body weight/dose to about 1 mg/kg body weight/dose, or about 0.2 mg/kg body weight/dose to about 0.5 mg/kg body weight/dose. In other embodiments the amount of calcium delivered to the upper respiratory tract (e.g., nasal cavity) is about 0.01 mg/kg body weight/dose to about 60 mg/kg body weight/dose, or about 0.01 mg/kg body weight/dose to about 50 mg/kg body weight/dose, about 0.01 mg/kg body weight/dose to about 40 mg/kg body weight/dose, about 0.01 mg/kg body weight/dose to about 30 mg/kg body weight/dose, about 0.01 mg/kg body weight/dose to about 20 mg/kg body weight/dose, 0.01 mg/kg body weight/dose to about 10 mg/kg body weight/dose, about 0.1 mg/kg body weight/dose to about 10 mg/kg body weight/dose, or about 1 mg/kg body weight/dose to about 10 mg/kg body weight/dose, or about 0.01 mg/kg body weight/dose to about 1 mg/kg body weight/dose, or about 0.1 mg/kg body weight/dose to about 1 mg/kg body weight/dose.

In some embodiments, a salt formulation that comprises a sodium salt (e.g., sodium chloride) is administered in an amount sufficient to deliver a dose of about 0.001 mg Na⁺/kg body weight/dose to about 10 mg Na⁺/kg body weight/dose, or about 0.01 mg Na⁺/kg body weight/dose to about 10 mg Na⁺/kg body weight/dose, or about 0.1 mg Na⁺/kg body weight/dose to about 10 mg Na⁺/kg body weight/dose, or about 1.0 mg Na⁺/kg body weight/dose to about 10 mg Na⁺/kg body weight/dose, or about 0.001 mg Na⁺/kg body weight/dose to about 1 mg Na⁺/kg body weight/dose, or about 0.01 mg Na⁺/kg body weight/dose to about 1 mg Na⁺/kg body weight/dose, or about 0.1 mg Na⁺/kg body weight/dose to about 1 mg Na⁺/kg body weight/dose.

In some embodiments the amount of sodium delivered to the respiratory tract (e.g., lungs, respiratory airway) is about 0.001 mg Na⁺/kg body weight/dose to about 10 mg Na⁺/kg body weight/dose, or about 0.01 mg Na⁺/kg body weight/dose to about 10 mg Na⁺/kg body weight/dose, or about 0.1 mg Na⁺/kg body weight/dose to about 10 mg Na⁺/kg body weight/dose, or about 1.0 mg Na⁺/kg body weight/dose to about 10 mg Na⁺/kg body weight/dose, or about 0.001 mg Na⁺/kg body weight/dose to about 1 mg Na⁺/kg body weight/dose, or about 0.01 mg Na⁺/kg body weight/dose to about 1 mg Na⁺/kg body weight/dose, about 0.1 mg Na⁺/kg body weight/dose to about 1 mg Na⁺/kg body weight/dose, about 0.2 mg Na⁺/kg body weight/dose to about 0.8 mg Na⁺/kg body weight/dose, about 0.3 mg Na⁺/kg body weight/dose to about 0.7 mg Na⁺/kg body weight/dose, or about 0.4 mg Na⁺/kg body weight/dose to about 0.6 mg Na⁺/kg body weight/dose about 0.001 mg/kg body weight/dose to about 10 mg/kg body weight/dose, or about 0.01 mg/kg body weight/dose to about 10 mg/kg body weight/dose, or about 0.1 mg/kg body weight/dose to about 10 mg/kg body weight/dose, or about 1 mg/kg body weight/dose to about 10 mg/kg body weight/dose, or about 0.001 mg/kg body weight/dose to about 1 mg/kg body weight/dose, or about 0.01 mg/kg body weight/dose to about 1 mg/kg body weight/dose, or about 0.1 mg/kg body weight/dose to about 1 mg/kg body weight/dose.

In some embodiments the amount of sodium delivered to the respiratory tract (e.g., lungs, respiratory airway) is about 0.001 mg/kg body weight/dose to about 10 mg/kg body weight/dose, or about 0.01 mg/kg body weight/dose to about 10 mg/kg body weight/dose, or about 0.1 mg/kg body weight/dose to about 10 mg/kg body weight/dose, or about 1 mg/kg body weight/dose to about 10 mg/kg body weight/dose, or about 0.001 mg/kg body weight/dose to about 1 mg/kg body weight/dose, or about 0.01 mg/kg body weight/dose to about 1 mg/kg body weight/dose, or about 0.1 mg/kg body weight/dose to about 1 mg/kg body weight/dose.

In other embodiments the amount of sodium delivered to the upper respiratory tract (e.g., nasal cavity) is about 0.001 mg Na⁺/kg body weight/dose to about 10 mg Na⁺/kg body weight/dose, or about 0.01 mg Na⁺/kg body weight/dose to about 10 mg Na⁺/kg body weight/dose, or about 0.1 mg Na⁺/kg body weight/dose to about 10 mg Na⁺/kg body weight/dose, or about 1.0 mg Na⁺/kg body weight/dose to about 10 mg Na⁺/kg body weight/dose, or about 0.001 mg Na⁺/kg body weight/dose to about 1 mg Na⁺/kg body weight/dose, or about 0.01 mg Na⁺/kg body weight/dose to about 1 mg Na⁺/kg body weight/dose, about 0.1 mg Na⁺/kg body weight/dose to about 1 mg Na⁺/kg body weight/dose, about 0.2 mg Na⁺/kg body weight/dose to about 0.8 mg Na⁺/kg body weight/dose, about 0.3 mg Na⁺/kg body weight/dose to about 0.7 mg Na⁺/kg body weight/dose, or about 0.4 mg Na⁺/kg body weight/dose to about 0.6 mg Na⁺/kg body weight/dose about 0.001 mg/kg body weight/dose to about 10 mg/kg body weight/dose, or about 0.01 mg/kg body weight/dose to about 10 mg/kg body weight/dose, or about 0.1 mg/kg body weight/dose to about 10 mg/kg body weight/dose, or about 1 mg/kg body weight/dose to about 10 mg/kg body weight/dose, or about 0.001 mg/kg body weight/dose to about 1 mg/kg body weight/dose, or about 0.01 mg/kg body weight/dose to about 1 mg/kg body weight/dose, or about 0.1 mg/kg body weight/dose to about 1 mg/kg body weight/dose.

In other embodiments the amount of sodium delivered to the upper respiratory tract (e.g., nasal cavity) is about 0.001 mg/kg body weight/dose to about 10 mg/kg body weight/dose, or about 0.01 mg/kg body weight/dose to about 10 mg/kg body weight/dose, or about 0.1 mg/kg body weight/dose to about 10 mg/kg body weight/dose, or about 1 mg/kg body weight/dose to about 10 mg/kg body weight/dose, or about 0.001 mg/kg body weight/dose to about 1 mg/kg body weight/dose, or about 0.01 mg/kg body weight/dose to about 1 mg/kg body weight/dose, or about 0.1 mg/kg body weight/dose to about 1 mg/kg body weight/dose.

Suitable intervals between doses that provide the desired therapeutic effect can be determined based on the severity of the condition (e.g., infection), overall well being of the subject and the subject's tolerance to the salt formulations and pharmaceutical compositions, and other considerations. Based on these and other considerations, a clinician can determine appropriate intervals between doses. Generally, a salt formulation or pharmaceutical composition is administered once, twice or three times a day, as needed.

### Administering Salt Formulation and Co-Therapeutic Formulations

In another aspect the invention relates to methods for treatment, prophylaxis and for reducing contagion of influenza or influenza-like illness (e.g., parainfluenza) comprising administering an effective amount of a salt formulation to the respiratory tract of an individual suspected of having influenza, with confirmed influenza, at risk for influenza, or with influenza-like illness, and administering an anti-influenza agent to the individual by any suitable route of administration. For example, the anti-influenza agent can be administered orally, parenterally (e.g., intravenous, intraarterial, intramuscular, or subcutaneous injection), topically, by inhalation (e.g., intrabronchial, intranasal or oral inhalation, intranasal drops), and the like. Any anti-influenza agent can be administered in accordance with this aspect of the invention, such as any one of the anti-influenza agents described herein. The salt formulation can be administered before, substantially concurrently with, or subsequent to administration of the anti-influenza agent. Preferably, the salt formulation and the anti-influenza agent are administered so as to provide substantial overlap of their pharmacologic activities. Advantageously, when a salt formulation as described herein and an anti-influenza agent are administered to an individual, so that there is overlap of their pharmacologic activities, the individual receives the beneficial effect of the anti-influenza agent and the beneficial effect of the salt formulation, which has its own therapeutic benefits and also can potentiate and synergize with the anti-influenza agent to produce superior therapy. In some embodiments, the salt formulation and the anti-influenza agent are administered in a synergistic effective amount.

In some embodiments, the method comprises administering an effective amount of a salt formulation (e.g., a formulation comprising a calcium salt and sodium chloride) to the respiratory tract of a patient with suspected of having influenza, with confirmed influenza, at risk for influenza, or with influenza-like illness, and also administering an effective amount of an NAI, an M2 channel inhibitor, an IMP dehydrogenase inhibitor, an influenza RNA polymerase inhibitor, a sialidase fusion protein, a sialyl multimer or polymer (e.g., sialylglycopolymer), an siRNA or oligonucleotide that targets expression of influenza genes, interferon alpha (e.g., PEGylated interferon alpha), an interferon inducer, such as double stranded RNA (poly(I)•poly(C)), or a signal transduction inhibitor to the individual by a suitable route of administration.

In particular embodiments, the method comprises administering an effective amount of a salt formulation (e.g., a formulation comprising a calcium salt and sodium chloride) to the respiratory tract of a patient suspected of having influenza, with confirmed influenza, at risk for influenza, or with influenza-like illness, and also administering an effective amount of zanamivir to the individual. Generally, the zanamivir is administered by inhalation.

In other embodiments, the method comprises administering an effective amount of a salt formulation (e.g., a formulation comprising a calcium salt and sodium chloride) to the respiratory tract of a patient suspected of having influenza, with confirmed influenza, at risk for influenza, or with influenza-like illness, and also administering an effective amount of oseltamivir or oseltamivir carboxylate to the individual. Generally, the oseltamivir or oseltamivir carboxylate is administered orally.

In some aspects, the invention is a therapeutic method that comprises administering to an individual suspected of having influenza or at risk of having influenza an effective amount of a calcium salt formulation and an effective amount of an anti-influenza agent, wherein the calcium salt formulation is administered to the respiratory tract. In some embodiments, the individual is suspected of having influenza and has one or more symptoms of influenza, such as fever and cough, or fever and sore throat. In other embodiments, the individual has influenza, e.g., is confirmed to be infected with influenza virus. Preferably, a synergistically effective amount of the calcium salt formulation and the anti-influenza agent are administered. Any suitable anti-influenza agent can be used, such as an NAI, an M2 channel inhibitor, an IMP dehydrogenase inhibitor, an influenza RNA polymerase inhibitor, a sialidase fusion protein, a sialyl multimer or polymer, an siRNA that targets expression of influenza genes, an oligonucleotide that targets expression of influenza genes, interferon alpha, an interferon inducer, and a signal transduction inhibitor, and combinations thereof. In some embodiments, the anti-influenza agent is selected from the group consisting of a NAI, a sialidase, a sialidase fusion protein and combinations thereof. In more particular embodiments an NAI, such as zanamivir, oseltamivir phosphate, and oseltamivir carboxylate and combinations thereof, is administered in accordance with the method.

In certain embodiment, influenza is caused by either the influenza A or the influenza B virus.

In certain embodiments, an influenza-like illness is caused by RSV, rhinovirus, adenovirus, parainfluenza, human coronaviruses (including the virus that causes severe acute respiratory syndrome) and metapneumovirus.

Any of the calcium salt formulations described herein can be administered in accordance with the method. For example, the calcium salt formulation can include a calcium salt selected from the group consisting of calcium chloride, calcium carbonate, calcium acetate, calcium phosphate, calcium alginate, calcium stearate, calcium sorbate, calcium sulfate, calcium gluconate, calcium lactate, calcium citrate and as combinations thereof. The calcium salt formulation can further comprise a sodium salt, such as a sodium salt selected from the group consisting of sodium chloride, sodium acetate, sodium bicarbonate, sodium carbonate, sodium sulfate, sodium stearate, sodium ascorbate, sodium benzoate, sodium biphosphate, sodium phosphate, sodium bisulfite, sodium citrate, sodium borate, sodium gluconate, sodium metasilicate, sodium lactate and the like, as well as combinations thereof. When the salt formulation contains a calcium salt and a sodium salt, the formulation can have any desired ratio of calcium:sodium, such as those ratios described here. Preferably the ratio of calcium to sodium in the calcium salt formulation is about 8:1 (mole:mole).

The therapeutic methods and uses of the invention provide particular benefits when the individual suspected of having influenza, with confirmed influenza, at risk for influenza, or with influenza-like illness also has a pulmonary disease, such as asthma (e.g., allergic/atopic, childhood, late-onset, cough-variant, or chronic obstructive), airway hyperresponsiveness, allergic rhinitis (seasonal or non-seasonal), bronchiectasis, chronic bronchitis, emphysema, chronic obstructive pulmonary disease, cystic fibrosis, early life wheezing, and the like. These patient populations are particularly susceptible to influenza, ILI and other respiratory infections, and these infections are frequent causes of acute exacerbation of the underlying pulmonary disease. Accordingly, the methods and therapeutic uses described herein can provide additional benefit in these patient populations by reducing the incidence, duration and/or severity of acute exacerbations of the underlying pulmonary disease.

### EXEMPLIFICATION

### Methods

A cell culture model of Influenza infection was used to study the effects of different nebulized salt formulations, anti-influenza agent formulations, or salt formulations that contained an anti-influenza agent on viral infection. Calu-3 cells (American Type Culture Collection, Manassas, Va.) were cultured on permeable membranes (12 mm Transwells; 0.4 μm pore size, Corning Lowell, Mass.) until confluent (the membrane was fully covered with cells) and air-liquid interface (ALI) cultures were established by removing the apical media and culturing at 37° C./5% CO₂. Cells were cultured for >2 weeks at ALI before each experiment. Prior to each experiment the apical surface of each Transwell was washed 3× with PBS (Hyclone, Logan, Utah). Cells were subsequently exposed to nebulized formulations using a sedimentation chamber and Series 8900 nebulizers (Slater Labs). Immediately after exposure, the basolateral media (media on the bottom side of the Transwell) was replaced with fresh media. Triplicate wells were exposed to each formulation in each test. A second cell culture plate was exposed to the same formulations to quantify the delivery of total salt or calcium to cells. One hour after exposure, cells were infected with 10 μL of Influenza A/WSN/33/1 at a multiplicity of infection of 0.1-0.01 (0.1-0.01 virions per cell). Four hours after aerosol treatment, the apical surfaces were washed to remove excess formulation and unattached virus and cells were cultured for an additional 20 h at 37° C. plus 5% CO₂. Twenty-four hours after aerosol treatment, virus released onto the apical surface of infected cells was collected in culture media or PBS and the concentration of virus in the apical wash was quantified by TCID₅₀ (50% Tissue Culture Infectious Dose) assay. The TCID₅₀ assay is a standard endpoint dilution assay that is used to quantify how much of a virus is present in a sample.

### Example 1

### Preparation of Test Formulations

1.3% CaCl₂ in 0.9% NaCl was made by dissolving 1.7 g of calcium chloride dihydrate (Spectrum Chemicals, Gardena, Calif.) in isotonic saline (0.9% NaCl; Cardinal Health, McGraw Park, Ill.) for a final concentration of 1.3% CaCl₂ in 0.9% NaCl. Liquid formulations of calcium chloride and sodium chloride at an 8:1 molar ratio of calcium:sodium (mole:mole) and at different tonicities were made by dissolving the appropriate amounts of each dry powder in sterile deionized (DI) water. The specific concentrations of each solution are shown in Table 2.

A stock solution of zanamivir was made by dissolving 25 mg of Relenza® dry powder consisting of 20 mg lactose and 5 mg zanamivir in sterile phosphate buffered saline (PBS).

A 10 mM stock solution of oseltamivir was made by dissolving 51.25 mg of oseltamivir dry powder (Roche #U2073) in 12.5 mL sterile PBS.

A 10 mM stock solution of Ribavirin was made by dissolving 12.2 mg of Ribavirin dry powder (Sigma #R9644) in 5 mL sterile PBS.

A 10 mM stock solution of Rimantadine was made by dissolving 10.86 mg of Ribavirin dry powder (Sigma # 390593) in 5 mL sterile PBS.

A 0.15 U/mL solution of sialidase was made by serially diluting a stock vial (50U/μL; New England Biolabs #P0720S) 1:10,000 in either sterile PBS or 1×G1 Buffer (50 mM Sodium citrate; New England Biolabs #B17235).

Solutions were subsequently diluted in either PBS or the appropriate Ca:Na salt formulations to the desired concentration (0.01 to 1 nM zanamivir; 1 nM oseltamivir; 1 or 10 nM of rimantadine; or 10 nM Ribavirin) and stored at 4° C. until use.

TABLE 2

Formulation

| Tonicity | CaCl2 | | NaCl | |
|---|---|---|---|---|
| 1X = isotonic | (% w/v) | CaCl2 (M) | (% w/v) | NaCl (M) |
| 0.5X | 0.59 | 0.053 | 0.04 | 0.007 |
| 1X | 1.2 | 0.11 | 0.08 | 0.013 |
| 2X | 2.4 | 0.21 | 0.16 | 0.027 |
| 4X | 4.7 | 0.43 | 0.31 | 0.053 |
| 8X | 9.4 | 0.85 | 0.62 | 0.11 |

Dry powder formulations were made with leucine (Spectrum Chemicals, Gardena, Calif.), calcium chloride dihydrate (Spectrum Chemicals, Gardena, Calif.), sodium chloride (Sigma-Aldrich Co., St. Louis, Mo.) and the neuraminidase inhibitor zanamivir (Relenza; GlaxoSmithKline, Research Triangle Park, N.C.) and were prepared on a spray dryer (Büchi B-290 mini spray dryer; New Castle, Del.). The system used a dehumidifier (Büchi B-296 dehumidifier; New Castle, Del.) to ensure stable temperature and humidity of the process drying air. Two feed solutions were prepared with the following components and ratios (weight percentage):
1) Leucine: Sodium Chloride: Zanamivir: Lactose 50.0000:49.9990:0.0019:0.0076
2) Leucine: Calcium Chloride: Sodium Chloride: Zanamivir: Lactose 50.000:29.730:20.265:0.001:0.003

Both solutions had a solids concentration of 5 g/L, where 1.25 g of salts and excipient were dissolved in 250 mL of deionized (DI) water. To add such a small amount of zanamivir, one 5 mg dose of zanamivir was dissolved into 1 L of DI water and the appropriate volume of this solution was added to the rest of the formulation. The formulations were each spray dried using the settings: inlet temperature of 220° C., outlet temperature 103-107° C., liquid flow rate of approximately 10 mL/min, room conditions at 24.4° C. and 17% RH, and dehumidifier air at 3-5° C. and 30% RH. The standard cyclone was used with the aspirator settings at 100%.

A third dry powder formulation containing leucine, calcium chloride dihydrate and sodium chloride was previously manufactured using similar processes. This dry powder consisted of leucine, calcium chloride, and sodium chloride at ratios of 50:29.5:20.5 (wt %).

For the delivery of formulations, capsules (QUALI-V-I, hypromellose, Size 2; Qualicaps, Europe S.A., Madrid, Spain) were filled with different amounts of each formulation such that equivalent amounts of zanamivir and calcium would be delivered with each formulation. The weight of each capsule before and after exposure was recorded to determine the emitted dose for each capsule preparation. Capsules were punctured with a 2-prong puncture fork and immediately loaded into a dry powder inhaler fitted to a dry powder sedimentation chamber. Dry powder was pulled into the sedimentation chamber from capsules using an automated vacuum system in which the vacuum was turned on for 0.3 seconds in three sequential intervals spaced 1 minute apart. Infections and washes were performed as described above for liquid formulations.

### Example 2

### Dry Powder Ca:Na Formulations Reduce Multiple Strains of Influenza Infection Dose-Dependently

Dry powders were made with leucine, a calcium salt (lactate or chloride), and sodium salt (chloride, sulfate, citrate or carbonate). Dry powders listed as 1 through 3 below were spray dried on a Büchi B-290 mini spray dryer. The system used the Büchi B-296 dehumidifier to ensure stable temperature and humidity of the air used to spray dry. Dry powder 4 was spray dried on a Niro Mobile Minor Spray Dryer in an open cycle with nitrogen.

Four liquid feed stocks were prepared with the following components and ratios (weight percentage):

1) Leucine: Calcium Lactate: Sodium Chloride//50:37:13

2) Leucine: Calcium Chloride: Sodium Sulfate//50:22:28

3) Leucine: Calcium Chloride: Sodium Citrate//50:19.5:30.5

4) Leucine: Calcium Chloride: Sodium Carbonate//50:25.5:24.4

Solutions 1-3 had a solids concentration of 5 g/L, while Solution 4 had a solids concentration of 2.5 g/L. The exact amount of salts and excipient dissolved in deionized (DI) water and its specific volume varied.

Dry Powders 1-3 were spray dried on the Büchi B-290 spray dryer with the following settings: inlet temperature of 220° C., liquid flow rate of approximately 10 mL/min, room conditions at 23.2-24.6° C. and 19-21% RH, and dehumidifier air at 3-5° C. and 30% RH. The outlet temperature, cyclone and aspirator rate varied. The dry powder in 1) was spray dried using a high performance cyclone with the aspirator at 80% and an outlet temperature of 93° C. Dry powders 2) and 3) were made with the regular cyclone, an aspirator at 100% and an outlet temperature of 111-115° C. Formulation 4) was spray dried on the Niro Mobile Minor spray dryer with the following settings: inlet temperature of 140° C., outlet temperature of 75° C., liquid flow rate of 30 mL/min, atomization gas rate of 30 g/min, drying gas flow rate of 100 kg/hr and drying chamber pressure of −2″WC.

To evaluate the efficacy of dry powder formulations, the Influenza viral replication model was used. This model utilizes Calu-3 cells grown at air-liquid interface as a model of influenza infection of airway epithelial cells. Calu-3 cells were exposed to dry powders using a dry powder sedimentation chamber. In order to expose cells to equivalent doses of calcium, capsules were filled with different amounts of each dry powder. The high, medium, and low fill weights were calculated based on matching the amount of calcium delivered by each dry powder (4.23 mg, 1.06 mg, and 0.35 mg). For each dry powder condition tested, two capsules were weighed as empty, filled, and after exposure in order to determine emitted dose of the dry powder. Table 3 shows the capsule fill weights before and after exposure and the concentration of calcium delivered to cells as determined by HPLC measurements.

One hour after exposure, cells were infected with 10 μL of Influenza A/WSN/33/1 or Influenza A/Panama/2007/99 at a multiplicity of infection of 0.1-0.01 (0.1-0.01 virions per cell). Four hours after dry powder treatment, the apical surfaces were washed to remove excess formulation and unattached virus and cells were cultured for an additional 20 hours at 37° C. plus 5% CO2. The next day (24 hours after infection) virus released onto the apical surface of infected cells was collected in culture media and the concentration of virus in the apical wash was quantified by TCID₅₀ (50% Tissue Culture Infectious Dose) assay. The TCID₅₀ assay is a standard endpoint dilution assay that is used to quantify how much of a given virus is present in a sample.

TABLE 3
Dry powder formulations tested to evaluate their effect on
Influenza A/WSN/33/1 infection in a cell culture model.

| | | | | | | Calcium |
|---|---|---|---|---|---|---|
| | | | | | Capsule | concentration |
| | | Intended | Empty | Filled | after | determined |
| Dry powder | | Fill | Capsule | Capsule | Exposure | by HPLC |
| Formulation | Lot # | (mg) | (mg) | (mg) | (mg) | (μg/cm²) |
| 50 Leucine::22 | 27.155.1 | 53.18 | 31.7 | 83.0 | 31.9 | 20.489 ± 0.745^{a} |
| Calcium Chloride::28 | 27.155.1 | 13.29 | 32.5 | 45.9 | 33.9 | 5.7560^{b} |
| Sodium Sulfate | 27.155.1 | 4.43 | 33.3 | 38.4 | 33.9 | 2.7950^{b} |
| 50 Leucine::37 | 45.6.1 | 62.17 | 64.972, | 99.649, | 64.994, | 50.8500 ± 1.114^{a} |
| Calcium Lactate::13 | | | 63.122* | 98.881* | 63.679* | |
| Sodium Chloride | 45.6.1 | 15.54 | 63.525 | 81.926 | 68.141 | 12.740 ± 1.702^{a} |
| | 45.6.1 | 5.18 | 62.453 | 67.796 | 62.49 | 3.9800^{b} |
| 50 Leucine::19.5 | 27.156.1 | 60.0 | 64.4 | 123.6 | 81.994 | 20.489 ± 5.720^{a} |
| Calcium Chloride::30.5 | 27.156.1 | 14.99 | 64.0 | 78.5 | 65.388 | 7.550 ± 0.880^{a} |
| Sodium Citrate | 27.156.1 | 5.00 | 63.5 | 70.3 | 63.829 | 3.610 ± 1.49^{a} |
| 50 Leucine::25.5 | 26.019.1 | 45.88 | 64.6 | 104.7 | 66.685 | 28.0600 ± 7.327^{a} |
| Calcium Chloride::24.5 | 26.019.1 | 11.47 | 61.5 | 72.0 | 63.186 | 8.1330 ± 2.582^{a} |
| Sodium Carbonate | 26.019.1 | 3.82 | 61.8 | 62.6 | 63.341 | 5.628 ± 2.696^{a} |

A. Dry Powder Formulations Reduce Influenza A/WSN/33/1 Infection in a Dose-Dependent Manner

Calu-3 cells were exposed to four different dry powder formulations each consisting of 50% leucine, a calcium salt and sodium chloride. Viral infection was assessed by quantifying the amount of viral replication over a 24 hour period. The specific dry powders tested are listed above in Table 3 and included carbonate, lactate, sulfate and citrate salts. To expose cells to approximately equivalent amounts of calcium of each of the four calcium containing dry powders, capsules were filled to appropriate fill weights prior to dosing. Cells exposed to no formulation (Air) were used as control cells.

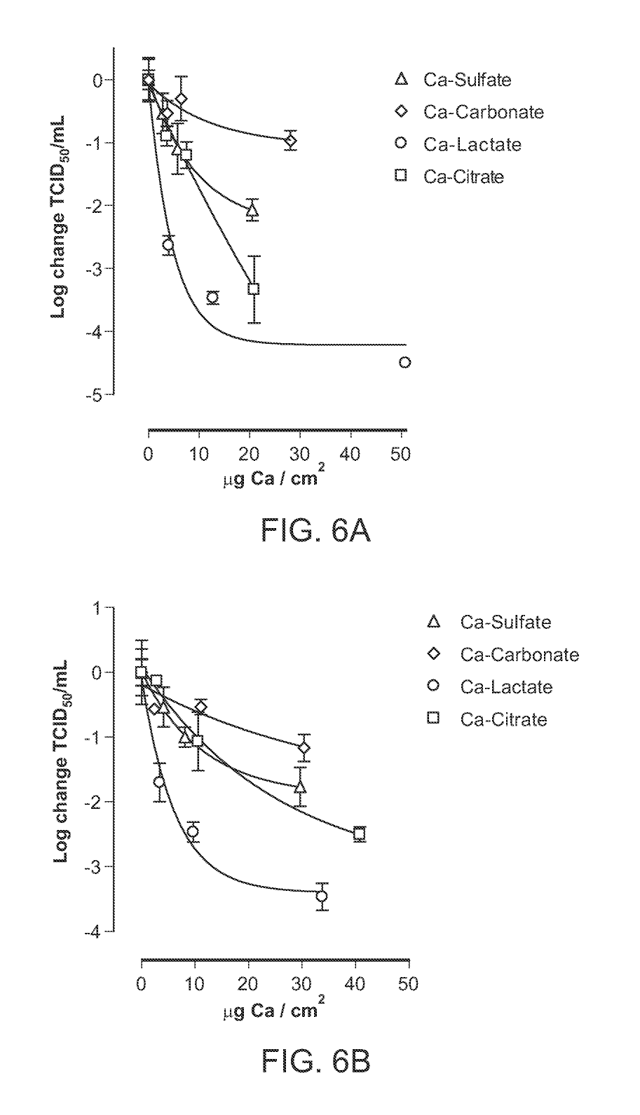

Each dry powder exhibited a dose-responsive reduction in influenza infection, however, the magnitude of the effect was different among the four dry powders tested (FIG. 6A). At low calcium concentrations calcium lactate was most efficacious, suggesting that it was the most potent of the dry powders tested. At higher concentrations of calcium, the calcium lactate and calcium citrate dry powders exhibited similar efficacy. The calcium sulfate dry powder exhibited an intermediate effect and was comparable to calcium citrate at several concentrations. Calcium carbonate had only a minimal effect on viral replication even at the highest concentration (less than 10-fold). Of note, calcium carbonate is the least soluble of the dry powders tested.

B. Dry Dry Powder Formulations Reduce Influenza A/Panama/2007/99 Infection in a Dose-Dependent Manner

The same dry powders were tested with a second influenza strain, Influenza A/Panama/2007/99 (H3N2). As described above, Calu-3 cells were exposed to four different dry powder formulations each consisting of 50% leucine, a calcium salt and sodium chloride. Viral infection was assessed by quantifying the amount of viral replication over a 24 hour period. The specific dry powders tested are listed in Table 4 and included carbonate, lactate, sulfate and citrate salts. To expose cells to approximately equivalent amounts of calcium of each of the four calcium containing dry powders, capsules were filled to appropriate fill weights prior to dosing. Cells exposed to no formulation (Air) were used as control cells.

Similar inhibition of viral replication was observed for each dry powder: calcium lactate was the most effective, calcium citrate and calcium sulfate exhibited intermediate efficacy and the calcium carbonate dry powder was only minimally efficacious (FIG. 6B). These data show that the Ca:Na dry powder formulations have anti-viral activity against multiple influenza strains.

TABLE 4
Dry dry powder formulations tested to evaluate their effect on
Influenza A/Panama/99/2007 infection in a cell culture model

| | | | | | | Calcium |
|---|---|---|---|---|---|---|
| | | | | | Capsule | concentration |
| | | Desired | Empty | Filled | after | determined |
| Dry powder | | Fill | Capsule | Capsule | Exposure | by HPLC |
| Formulation | Lot # | (mg) | (mg) | (mg) | (mg) | (μg/cm² ± SD) ^{a} |
| 50 Leucine::22 | 27.155.1 | 53.18 | 61.358 | 121.417 | 62.591 | 40.780 ± 4.99 |
| Calcium Chloride::28 | 27.155.1 | 13.29 | 60.602 | 76.804 | 62.167 | 10.544 ± 2.25 |
| Sodium Sulfate | 27.155.1 | 4.43 | 65.102 | 70.789 | 65.670 | 2.870 ± 0.61 |
| 50 Leucine::37 | 45.6.1 | 62.17 | 64.037 | 125.465 | 67.043 | 33.770 ± 3.45 |
| Calcium Lactate::13 | 45.6.1 | 15.54 | 65.358 | 82.474 | 65.632 | 9.650 ± 1.42 |
| Sodium Chloride | 45.6.1 | 5.18 | 66.046 | 72.455 | 66.324 | 3.415 ± 0.924 |
| 50 Leucine::19.5 | 27.156.1 | 60.0 | 62.581 | 108.035 | 63.841 | 29.620 ± 10.12 |
| Calcium Chloride::30.5 | 27.156.1 | 14.99 | 63.393 | 75.770 | 64.085 | 8.100 ± 1.38 |
| Sodium Citrate | 27.156.1 | 5.00 | 65.910 | 70.062 | 66.204 | 4.090 ± 0.823 |
| 50 Leucine::25.5 | 26.019.1 | 45.88 | 64.506 | 115.876 | 65.004 | 30.380 ± 11.89 |
| Calcium Chloride::24.5 | 26.019.1 | 11.47 | 64.319 | 77.627 | 65.080 | 11.070 ± 4.34 |
| Sodium Carbonate | 26.019.1 | 3.82 | 66.495 | 71.398 | 66.698 | 2.370 ± 1.01 |

### Example 3

### Ca:Na Liquid Formulations Improve the Course of Influenza Infection

Mouse Model

Mice (Balb/c) were treated with saline or calcium:sodium formulations at 8:1 molar ratios of Ca²⁺:Na⁺ at different tonicities (1×, 2×, 4× or 8×; Table 2) concentration beginning three hours before infection, three hours after infection and then BID for up to 11 days. Mice were lightly anesthetized with ketamine/xylazine and a lethal dose of virus (Influenza A/PR/8) was delivered intranasally. The primary endpoint of the study was animal survival for up to 21 days after infection. Animal body temperatures and body weights were monitored throughout the study. Animals with body temperatures below 95° F. were euthanized.

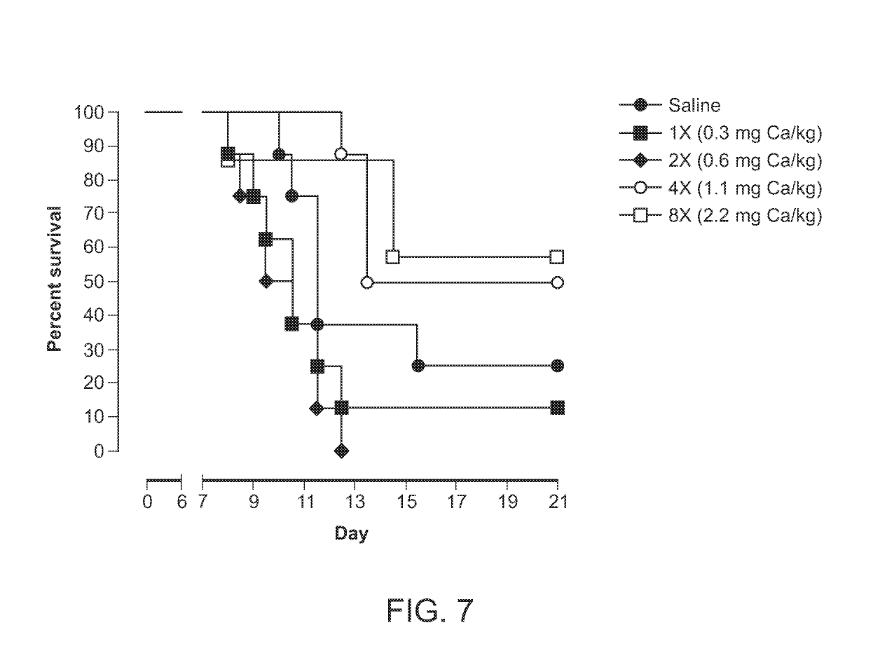

To study the effects of calcium:sodium (8:1 molar ratio of Ca²⁺:Na⁺) formulations on influenza infection, survival studies were performed. Mice were administered a lethal dose of influenza and treated with Ca:Na formulations at different tonicities. The dose time for each formulation was constant, resulting in increased doses of calcium as the tonicity of the formulation was increased. Saline treated animals were used as controls. FIG. 7 shows the survival data for each group over time. In this study, 75% of the control animals died before the end of the study on day 21. In contrast, 50% and 42.9% of the 4× and 8× treated animals died, demonstrating that treatment with these formulations improved the fraction of surviving animals.

Ferret Model

The ferret model of influenza is a standard model for the evaluation of influenza vaccines or antivirals. Using this model we tested the efficacy of 1.3% CaCl₂, 0.9% NaCl (Formulation 1) and two formulations that were optimized in vitro for enhanced activity against influenza replication. The formulations tested are shown in Table 5. Control ferrets were exposed to inhalation grade water for the same duration (6.5 minutes) and under the same exposure conditions. Aerosol formulations were generated from two PariLC Sprint nebulizers and ferrets were exposed to nebulized formulations using a FlowPast exposure system (TSE systems). Ferrets were dosed 1 hour before infection, 4 hours after infection and then BID for 6 days until the termination of the study. Nasal wash samples were collected once daily beginning on day 1 of the study and body temperatures and body weights were determined twice a day beginning on day 0 of the study. The number of inflammatory cells and the viral titer in nasal wash samples were determined.

TABLE 5

| | Calcium | Sodium | | |
|---|---|---|---|---|
| | chloride | chloride | | Delivered |
| | concentration | concentration | Ratio | dose (mg |
| Formulation | (M) | (M) | Ca:Na | CaCl₂/kg)* |
| Formulation 1 | 0.116 | 0.15 | 1:1.3 | 0.56 |
| 4X | 0.424 | 0.053 | 8:1 | 1.99 |
| 8X | 0.849 | 0.106 | 8:1 | 3.44 |

*The delivered dose was determined from measurements made from the sample port of the nose-only exposure system and the calcium concentration was determined by HPLC methods.

Ca:Na Formulations Prevent the Onset and Severity of Fever

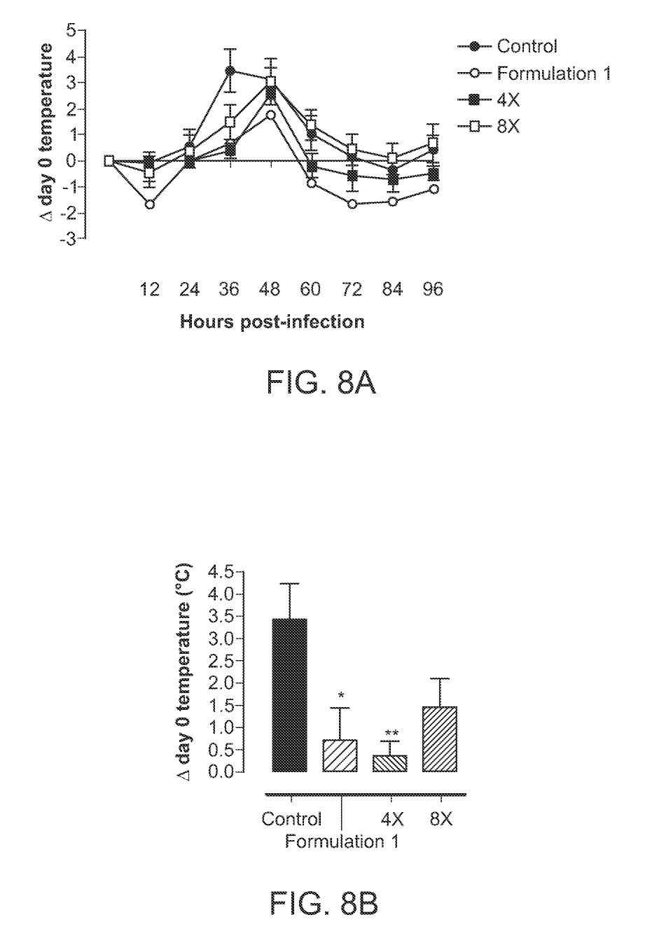

Compared to control ferrets, 1.3% CaCl₂, 0.9% NaCl (Formulation 1), 4× and 8× treated ferrets exhibited a delayed onset of fever and reduced peak body temperature relative to the control group (FIG. 8). At the time of peak fever (36 hours post infection) the 1.3% CaCl₂, 0.9% NaCl (Formulation 1) and 4× treatment groups exhibited significantly reduced body temperatures compared to the control group (mean increase of 3.4° C. in the control group compared to 0.7° C. in the 1.3% CaCl₂, 0.9% NaCl (Formulation 1) and 0.4° C. in the 4× group; p<0.05 and p<0.01, respectively Mann-Whitney U test; FIG. 8). Notably, the 8× treatment group also exhibited reduced body temperatures compared to control animals (mean increase 1.45° C.), however, the difference was not statistically significant (p=0.065). These data suggest that Ca:Na formulation can effectively reduce the severity and onset of fever following influenza infection in ferrets.

Ca:Na Formulations Reduce Body Weight Loss.

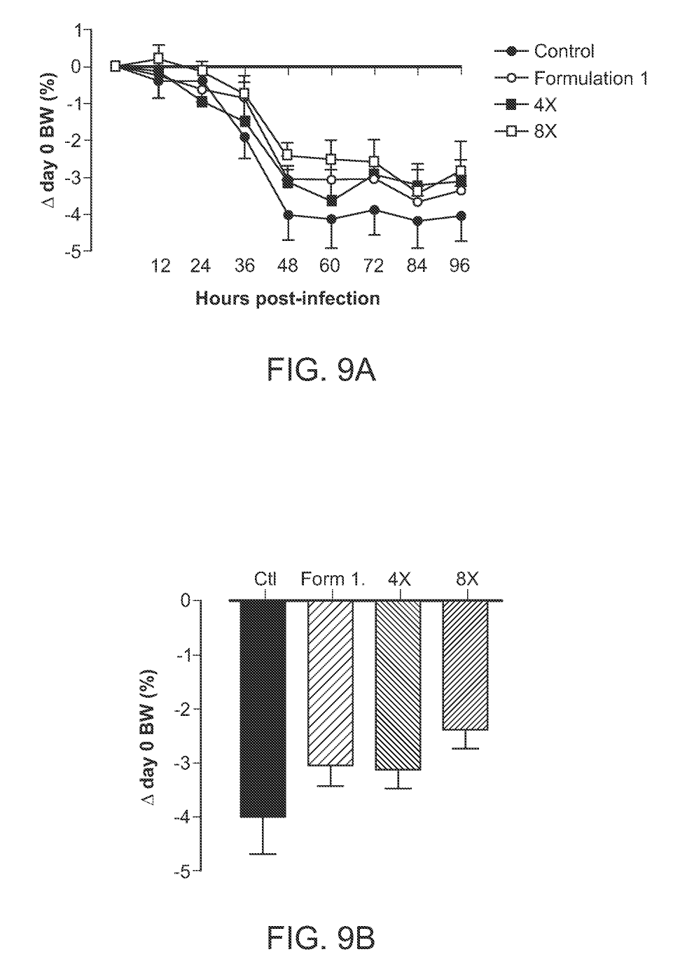

Control ferrets lost weight more rapidly and exhibited a greater percentage of body weight loss 48 hours post-infection compared to treated animals (FIG. 9). The body weight loss in the 4× and 8× groups was statistically significant from control animals (4.0% weight loss in the control group compared to 3.1% and 2.4% in the 4× and 8× groups, respectively; FIG. 9). The data also suggest a dose-responsive reduction of weight loss as the 8× treated animals lost the least amount of weight.

Ca:Na Formulations Reduce Nasal Inflammatory Cell Counts.

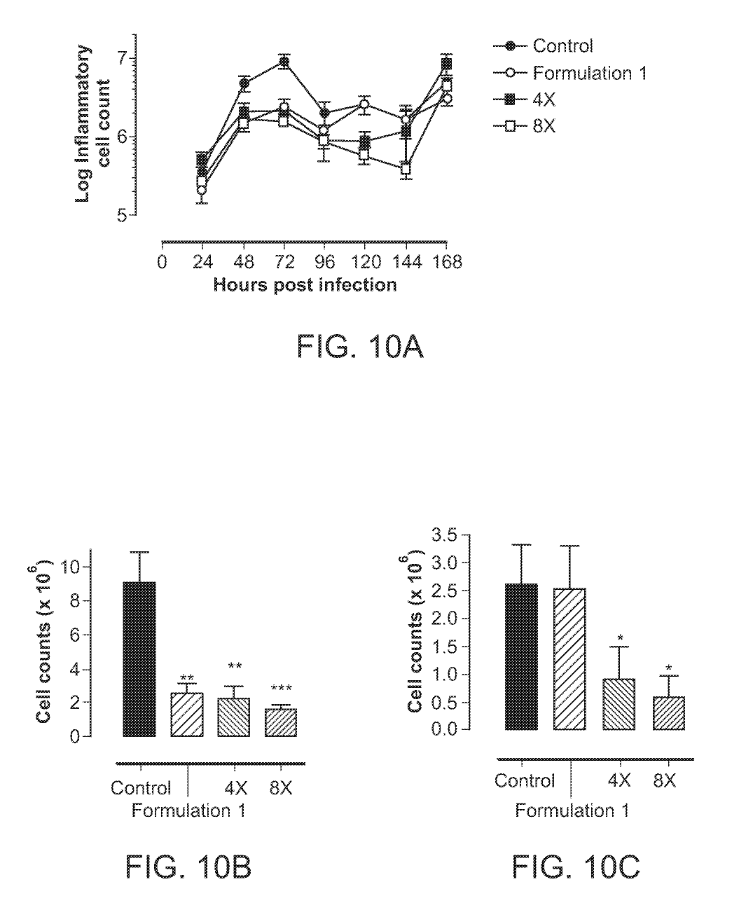

Influenza infection in the upper airways is associated with an infiltration of inflammatory cells aimed at resolving the infection. This inflammation is also a primary cause of the clinical symptoms associated with infection. To determine if Ca:Na formulation treatment reduced inflammation following influenza infection, the number of inflammatory cells in nasal washes from control or treated ferrets were determined. Inflammatory cell counts were significantly lower in the Ca:Na formulation treated groups compared to the control group over the course of the study (FIG. 10; p<0.0001 Two-way ANOVA). The total number of inflammatory cells recovered from 1.3% CaCl₂, 0.9% NaCl (Formulation 1), 4× and 8× treated ferrets was significantly lower than in control ferrets 72 hours after infection (p<0.01 for 1.3% CaCl₂, 0.9% NaCl (Formulation 1) and 4×; p<0.001 for the 8× treatment; Mann-Whitney U test). Furthermore, the 4× and 8× treatments resulted in a statistically significant difference in inflammatory cell counts 120 hours after infection (p<0.05 Mann-Whitney U test). The latter result suggests that at higher doses of Ca:Na formulation treatment, inflammation may be inhibited for longer periods of time. Collectively, these data demonstrate that Ca:Na formulations reduce the clinical symptoms and dampen the inflammatory response associated with influenza infection.

Ca:Na formulation treatments improve the clinical course of influenza infection and dampen the inflammatory response to influenza infection in ferrets. Differences in body weight loss and inflammatory cell counts suggest that increased doses of calcium chloride can be associated with improved outcomes.

### Example 4

The combination of calcium and zanamivir is more effective at reducing influenza infection than either compound alone.

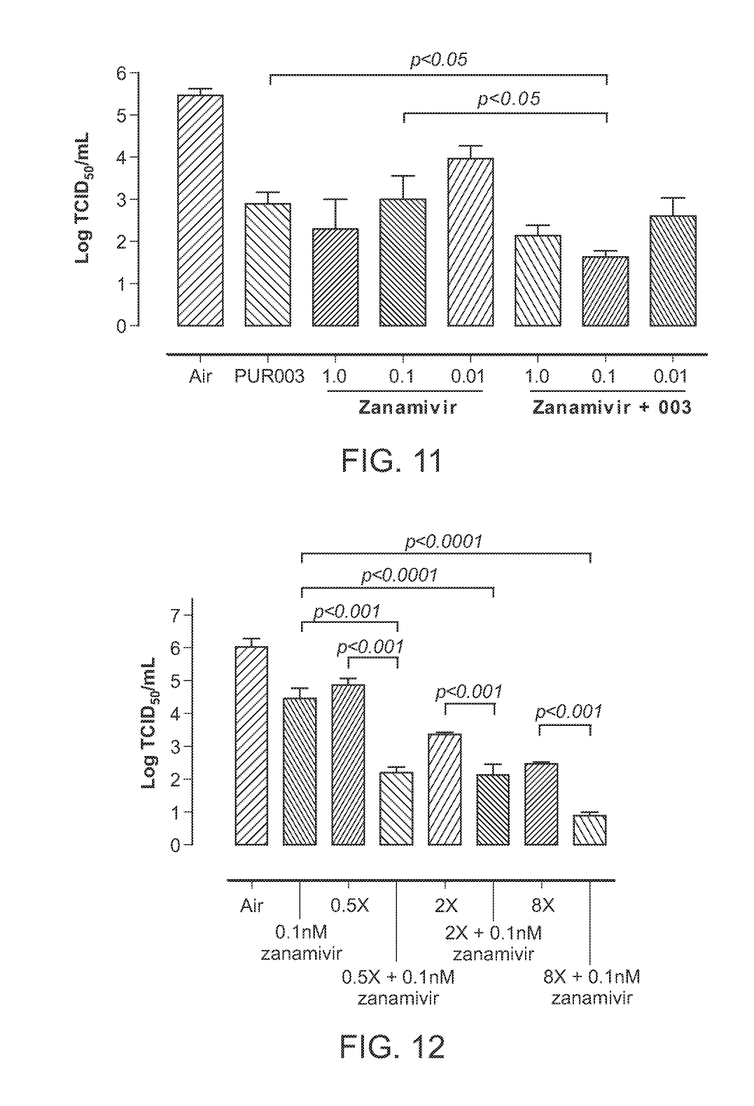

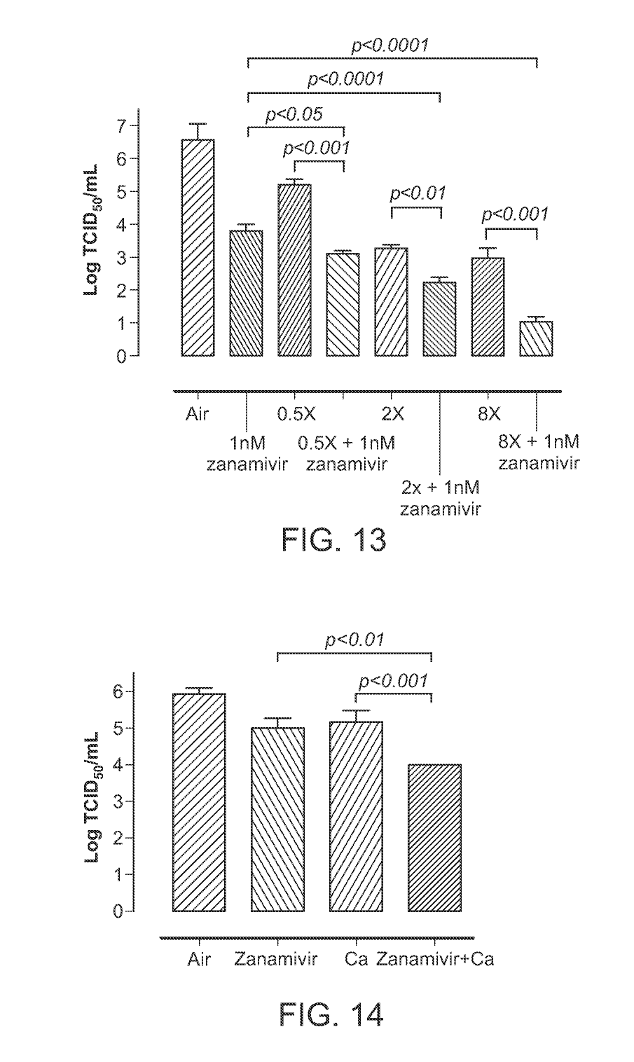

Calu-3 cells were exposed to liquid aerosols of either zanamivir (0.01 to 1.0 nM in PBS) or 1.3% CaCl₂ in 0.9% saline and infected with Influenza A/WSN/33/1 1 h after exposure. The viral titer on the apical surface of cells was determined 24 h after dosing. Zanamivir reduced viral infection in a dose responsive manner (p<0.01 compared to untreated (Air) control; one way ANOVA with Tukey's multiple comparison test). Similarly, 1.3% CaCl₂ in 0.9% NaCl significantly reduced viral titers approximately 300-fold compared to untreated controls, a level that was comparable to the 0.1 nM concentration of zanamivir (FIG. 11).

To test whether the combination of zanamivir and calcium would further reduce viral infection over zanamivir or 1.3% CaCl₂ in 0.9% NaCl alone, Calu-3 cells were exposed to the same concentrations of zanamivir in 1.3% CaCl₂ in 0.9% NaCl. The combination formulations each significantly reduced Influenza titers compared to the untreated controls (p<0.001 compared to untreated (Air) control; one way ANOVA with Tukey's multiple comparison test). Statistical analysis of the data (one way ANOVA with Tukey's multiple comparison test) revealed that the combination of 1.3% CaCl₂ in 0.9% NaCl with 0.01 nM zanamivir resulted in a statistically significant reduction in viral infection compared to the single treatment of 1.3% CaCl₂ in 0.9% NaCl or the matched zanamivir concentration. The combined treatment effect was ˜20-fold greater than either 0.1 nM zanamivir or 1.3% CaCl₂ in 0.9% NaCl alone (FIG. 11) showing that the combination of 1.3% CaCl₂ in 0.9% NaCl and 0.01 nM zanamivir provided the greatest reduction in viral titer.

### Example 5

The combination of zanamivir with different concentrations of CaCl₂ is more effective at reducing influenza infection than either compound alone.

To determine if the combined effects of calcium and zanamivir were specific to 1.3% CaCl₂ in 0.9% NaCl and 0.1 nM zanamivir or could be generalized to other calcium concentrations, dose response studies of CaCl₂ in NaCl with and without zanamivir were performed. Calcium formulations containing calcium and sodium at an 8:1 molar ratio of calcium:sodium (mole:mole), but at different tonicities (and therefore different calcium concentrations), reduced influenza infection in a dose-responsive manner. The 0.5×, 2×, and 8× formulations (see Table 2) reduced viral titers compared to the untreated control by approximately 16-, 400-, and 3000-fold, respectively (FIG. 12). Exposure of cells to 0.1 nM zanamivir similarly reduced viral titers 40-fold compared to the untreated control. When 0.1 nM zanamivir was delivered in combination with each of the calcium formulations, a greater reduction in viral titer was observed compared to each of the matched single treatments (FIG. 12).

The fold-difference in Influenza titer for each combination treatment compared to the respective single treatment was calculated and shown in Table 6. Unexpectedly, the 0.5× formulation with zanamivir exhibited comparable efficacy to the 2.0× formulation with zanamivir (Lines 1 and 3; Table 6). This was evident despite the finding that the 2× formulation without zanamivir was more than 30-fold more effective than the 0.5× formulation without zanamivir (FIG. 12). This is further evident by comparing the effect of the combined formulations with each of the calcium treatments alone. The 0.5× formulation plus zanamivir reduced viral titers more than 400-fold compared to the 0.5× treatment alone. The 2× formulation plus zanamivir was about 17-fold more effective than the 2× formulation in reducing viral titer (Line 2; Table 6). These findings show that the 0.5× salt formulation and zanamivir produced superior reduction in viral titer in comparison to other tested combinations.

TABLE 6
Fold-difference of combination treatments relative to [symbol]
similar relationship between the 0.5X formulation and [symbol]
of the

| | | 0.5X + | 2X + | 8X + |
|---|---|---|---|---|
| | | Zanamivir | Zanamivir | Zanamivir |
| Line | | (0.1 nM) | (0.1 nM) | (0.1 nM) |
| 1 | Untreated | 6,807 | 7,943 | 135,831 |
| | control | | | |
| 2 | Matched | 463 | 17 | 37 |
| | calcium | | | |
| | formulation | | | |
| 3 | 1.0 nM | 184 | 215 | 3,681 |
| | zanamivir | | | |

[symbol]

indicates data missing or illegible when filed

combination formulations tested significantly reduced viral titers compared to the untreated control, 1.0 nM zanamivir, and to each of the respective calcium formulations alone (Table 7). As seen with formulations that contained 0.1 nM zanamivir, the 0.5× formulation with 1 nM zanamivir exhibited the greatest reduction in titer compared to the respective calcium formulation alone (Line 2; Table 7). Likewise, the combination of 8× and 1.0 nM zanamivir was 86-fold more effective than the 8× formulation alone (Line 2; Table 7). The combination of 2× formulation with zanamivir was 11-fold more effective than the 2× formulation alone. Thus, some combinations of zanamivir with calcium chloride were more efficacious than others in reducing Influenza infection.

TABLE 7
Fold-difference of combination treatments relative to
single treatments

| | | 0.5X + | 2X + | 8X + |
|---|---|---|---|---|
| | | Zanamivir | Zanamivir | Zanamivir |
| Line | | (1 nM) | (1 nM) | (1 nM) |
| 1 | Untreated | 2,931 | 21,528 | 341,193 |
| | control | | | |
| 2 | Matched | 126 | 11 | 86 |
| | calcium | | | |
| | formulation | | | |
| 3 | 0.1 nM | 5 | 37 | 583 |
| | zanamivir | | | |

### Example 6

The combination of zanamivir and calcium chloride in dry powder form is more effective at reducing viral infection than either compound alone.

Zanamivir is typically delivered in dry powder form. To determine if the enhanced efficacy of calcium and zanamivir would be evident in dry powder formulations, dry powder formulations were prepared that consisted of zanamivir alone (with NaCl), calcium chloride alone (with NaCl), and the combination of the zanamivir and calcium chloride. The dry powder formulations consisting of either zanamivir alone or calcium chloride alone reduced Influenza titers to similar levels, 8.6- and 5.8-fold respectively. (FIG. 14) When zanamivir and calcium chloride were co-delivered in the same dry powder formulation, viral titers were further reduced. This reduction was 86-fold compared to the air-control and at least 10-fold greater than either of the single treatments alone. Thus, the combined effects of zanamivir and calcium chloride resulted in enhanced effectiveness in reducing influenza infection in both liquid and dry powder form.

### Example 7

Analysis of data on linear scale identifies synergistic combinations of calcium chloride and zanamivir.

Considering whether the combined effects are considered synergistic in nature, two models of additivism have been used (Borisy et al., Proc. Natl. Acad. Sci. USA 100: 7977-7982 (2003)). The highest single agent (HSA) model states that additivism is the larger of the effects produced by each of the single agents at the same concentration in the mixture. A second model to demonstrate addvitism is the Bliss additivism model. (Bliss, C. I., Ann. Appl. Biol., 26:585-615 (1939).) It predicts that the combined response C for two compounds with effects A and B is C=A+B−A×B, where each effect is expressed as the fractional inhibition. Each of these models was used with the data presented above using the data on a linear scale. This data is summarized in Tables 8 and 9. Using the HSA prediction, the combination of 0.1 nM zanamivir and calcium chloride exceeds additivism by greater than 20% at all three doses of calcium tested. Similarly, the 8× calcium formulation with 1.0 nM zanamivir exceeds this prediction model. Using the Bliss additivism prediction, the 0.5× calcium formulation with zanamivir exceeded the prediction model. This formulation also exceeded the HSA prediction to the greatest degree indicating that this combination exhibits synergistic activity.

TABLE 8
Excess inhibition over Highest Single Agent prediction

| | | | |
|---|---|---|---|
| Calcium | 8X | 26% | 29% |
| formulation | 2X | 21% | 16% |
| | 0.5X | 38% | 11% |
| | 0 | 0.1 nM | 1.0 nM |

Zanamivir concentration

TABLE 9
Excess inhibition over Bliss Addvitism prediction

| | | | |
|---|---|---|---|
| Calcium | 8X | 15% | −1% |
| formulation | 2X | 6% | −5% |
| | 0.5X | 24% | 10% |
| | 0 | 0.1 nM | 1.0 nM |

Zanamivir concentration

### Example 8

The combination of oseltamivir with different concentrations of CaCl₂ is more effective at reducing influenza infection than either compound alone.

To determine if the combined effects of calcium formulations and oseltamivir were more efficacious than either single treatment, cells were exposed to both therapies in combination or as standalone treatments and influenza viral titers were measured. The Ca:Na formulations were delivered by aerosol and the oseltamivir was delivered in the basolateral media of the Calu-3 cells. The 1 nM oseltamivir reduced influenza titers 5-fold compared to untreated (air) control and at higher concentrations of oseltamivir greater reductions in influenza infections were observed.

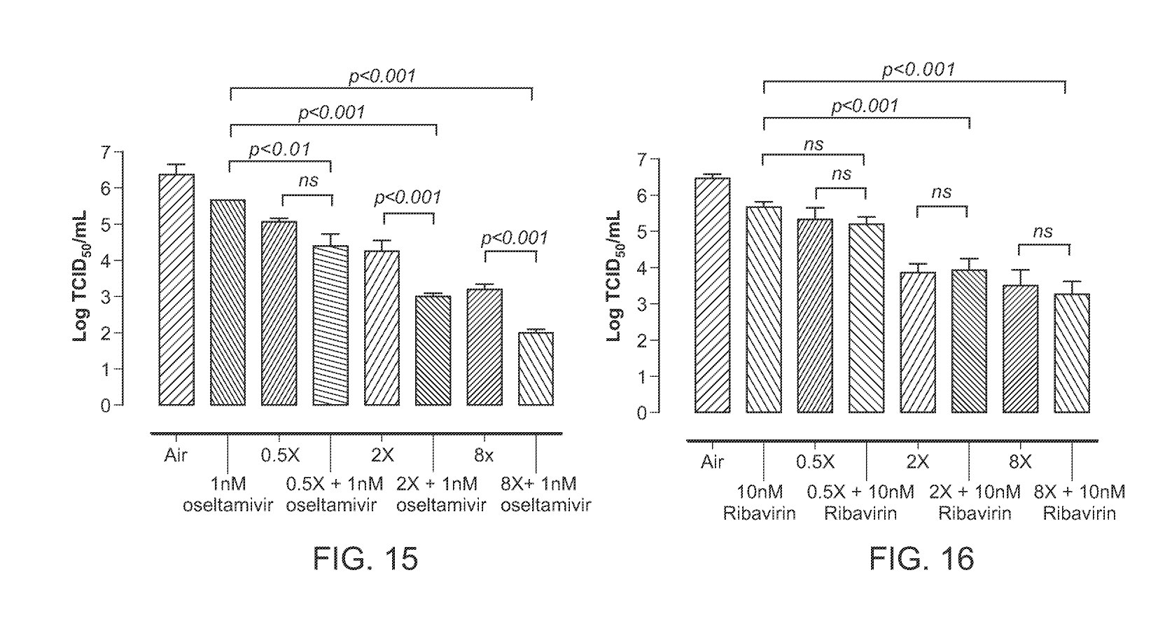

Formulations containing calcium and sodium at an 8:1 molar ratio, but at different tonicities (and therefore different calcium concentrations; Table 2), reduced Influenza infection in a dose-responsive manner. The combined treatment of oseltamivir with either the 8× or 2× formulations was 15.8- and 17.8-fold greater than the respective calcium formulation alone (FIG. 15; p<0.001; one-way ANOVA with Tukey's multiple comparison post-test). Surprisingly, a similar combination effect was not seen with the 0.5× formulation and oseltamivir. Previously, the 0.5× formulation was shown to act synergistically with zanamivir to reduce influenza infection. Together these data support the findings made with combinations of calcium and zanamivir and show that Ca:Na formulations can be used together with neuraminidase inhibitors to provide a maximal therapeutic benefit that cannot be attained with similar concentrations of the monotherapies.

### Example 9

The combined effects of 10 nM ribavirin and calcium show no enhanced efficacy over either treatment alone.

To determine if another class of antivirals resulted in a similar combination effect as the neuraminidase inhibitors with Ca:Na formulations, a similar study was performed with 10 nM ribavirin. Cells were exposed to a dose response of Ca:Na formulations at an 8:1 molar ratio with and without ribavirin added to the basolateral media. In this assay, ribavirin reduced influenza titers 6.3-fold compared to untreated (air) control. (FIG. 16; p>0.05 compared to untreated control; one-way ANOVA with Tukey's multiple comparison post-test). Each of the combination treatments were more efficacious than the ribavirin treatment alone, however, there was no statistical difference between any of the combination treatments with the respective Ca:Na exposure. Thus, any decrease in viral titer was driven primarily by the calcium formulations, showing that the addition of ribavirin does not increase the therapeutic effect of calcium formulations.

Ribavirin, rimantidine (See Example 12), and the neuraminidase inhibitors (zanamivir and oseltamivir) act through different mechanisms and inhibit influenza infection at different steps in the replication process. These differences in mechanism may account for the observed differences when used in combination with Ca:Na formulations.

### Example 10

Liquid combinations of sialidase and 2× 8:1 Ca:Na formulations (8:1 molar ratios) reduce influenza infection to a greater extent than either standard therapy.

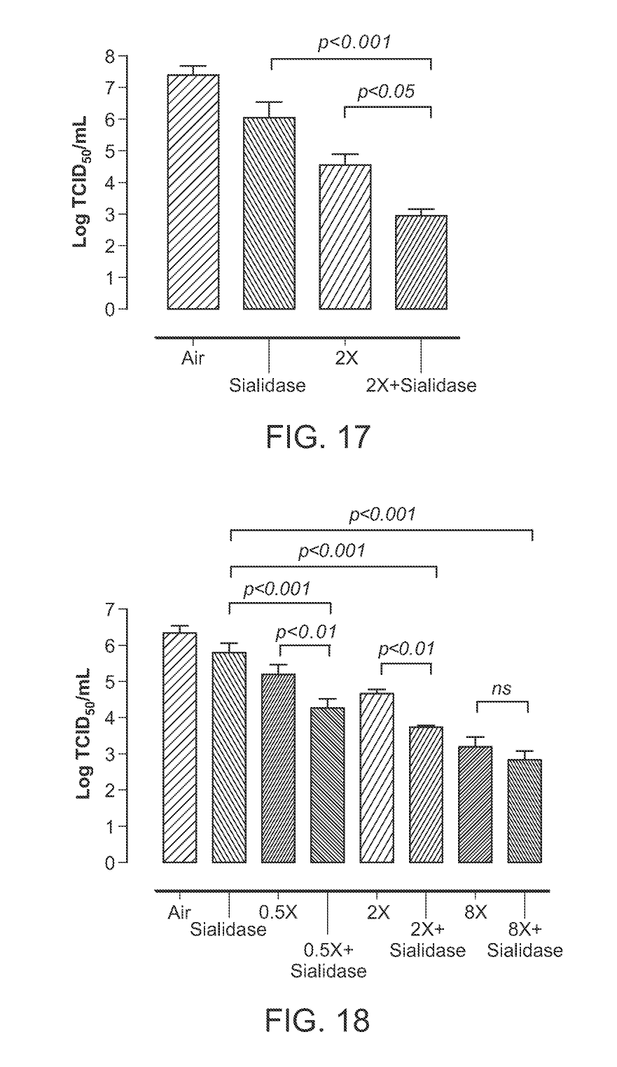

Initial experiments determined the effect of sialidase treatment on the viral replication cell culture model of Example 3. In this study cells were exposed to 0.150 U/mL of sialidase (New England Biolabs #P0720S) in G1 buffer (50 mM Sodium citrate, pH 6.0; New England Biolabs) with or without 2× Ca:Na formulation (Table 2). Cells were subsequently infected with Influenza A/Panama/2007/99 and viral titers were measured. The Ca:Na formulations were delivered by aerosol and the neuraminidase was applied to the apical surface of the Calu-3 cells. Treatment of cells with 0.150 U/mL sialidase alone significantly reduced influenza titer compared to untreated (air) control (p<0.05; one-way ANOVA with Tukey's multiple comparison post-test; FIG. 17).

To test whether the combined effects of sialidase and 2× Ca:Na formulation were more efficacious than either of the matched treatments alone, cells were exposed to both the 2× Ca:Na formulation treatment was combined with sialidase. This combination reduced Influenza infection 39.8-fold compared to the Ca:Na formulation alone (p<0.05; one-way ANOVA with Tukey's multiple comparison post-test) and 1259-fold compared to the sialidase treated cells (p<0.001; one-way ANOVA with Tukey's multiple comparison post-test). Together these data show that the combination of sialidase treatment with Ca:Na treatment exhibits a greater effect than either single treatment.

### Example 11

The combined effect of sialidase and specific 8:1 Ca:Na formulations reduce influenza infection to a greater extent than either standard therapy.

To determine if the combination effect of sialidase and 2× Ca:Na formulation also applied to combinations with other 8:1 Ca:Na formulations, dose response studies of CaCl₂ in NaCl formulations with and without sialidase were performed. In this study, specified cells were apically exposed to 0.150 U/mL of sialidase in appropriate G1 buffer with or without a dose response of Ca:Na formulations at an 8:1 molar ratio (Table 2). Cells were infected with Influenza A/Panama/2007/99 and viral titers were measured. The Ca:Na formulations were delivered by aerosol.

In this assay, both the 0.5× and 2× calcium formulations combined with sialidase reduced influenza titers 8.57-fold compared to their respective calcium formulation alone (FIG. 18; p<0.01; one-way ANOVA with Tukey's multiple comparison post-test). However, there was no statistical difference between the 8× calcium formulation combined with sialidase and the 8× formulation alone showing some specificity to the combination effects (p>0.05; one-way ANOVA with Tukey's multiple comparison post-test).

### Example 12

### Ca:Na Formulations Enhance the Efficacy of 10 nM Rimantadine

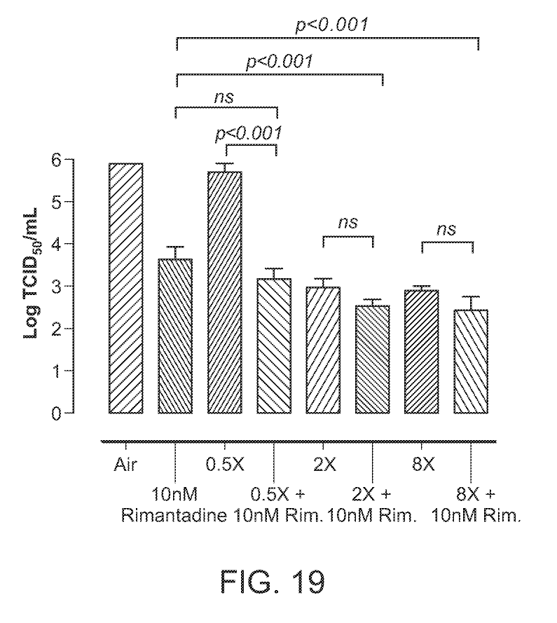

To determine if Ca:Na formulations could be combined with other anti-influenza therapies to provide enhanced efficacy, combinations of calcium formulations and rimantadine were tested. Cells were exposed to both therapies in combination or as standalone treatments and viral titers were measured. Ca:Na formulations were delivered by aerosol and the rimantadine was delivered in the basolateral media of the Calu-3 cells, similar to the protocol used for oseltamivir. Exposure of cells to 10 nM rimantadine reduced viral titers 185-fold compared to the untreated control (FIG. 19; p<0.001 compared to untreated control; one-way ANOVA with Tukey's multiple comparison post-test). When 10 nM rimantadine was added in combination with each of the 2× and 8× formulations, a greater reduction in viral titer was observed compared to the rimantadine alone treatment. However, none of these combinations were statistically different from the respective Ca:Na formulations (p>0.05; one-way ANOVA with Tukey's multiple comparison post-test; FIG. 19). Similarly, the combination of the 0.5× formulation and rimantidine significantly reduced viral titers compared to the 0.5× treatment alone, however this reduction was not statistically different from the rimantadine alone condition. Thus, none of the Ca:Na and rimantadine combination treatments provided an additive or synergistic benefit over the respective standalone treatments.

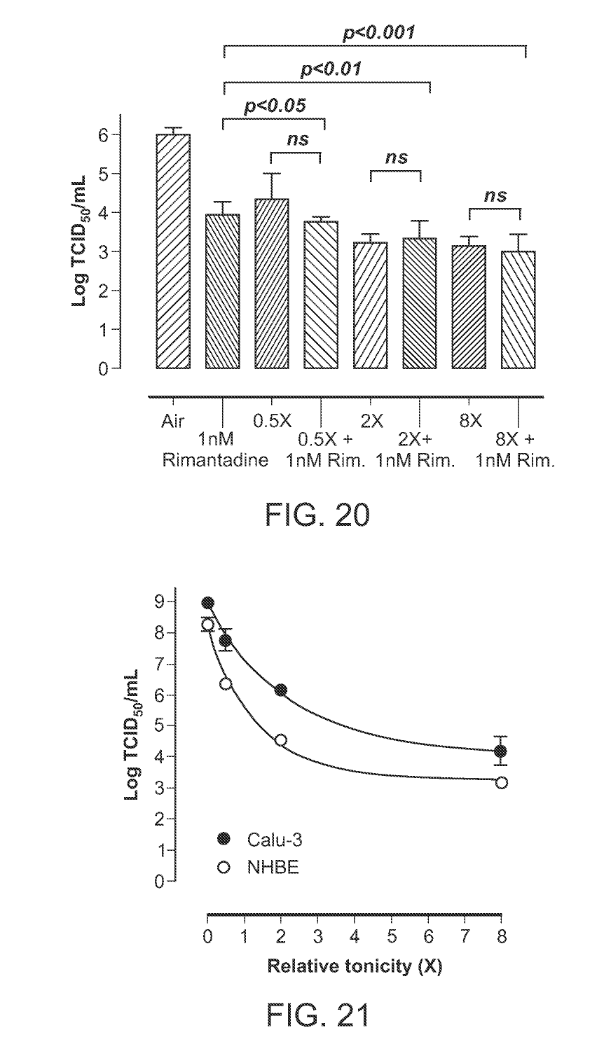

To further test this, a second concentration of rimantadine (1 nM) was tested in combination with the same Ca:Na formulations. Cells were exposed to a similar dose range of Ca:Na formulations with and without rimantadine added to the basolateral media. The lower concentration of rimantadine reduced influenza titers 9.6-fold compared to untreated (air) control and was less efficacious than the 10 nM concentration in the previous study (FIG. 20; p>0.05 compared to untreated control; one-way ANOVA with Tukey's multiple comparison post-test). Each of the combination treatments were more efficacious than the rimantadine treatment alone, however, there was no statistical difference between any of the combination treatments with the respective Ca:Na exposure (FIG. 20; p>0.05 compared to untreated control; one-way ANOVA with Tukey's multiple comparison post-test). These data suggest that the observed decrease in viral titer following the combination treatment was primarily caused by the calcium formulations. Thus, unlike combinations with NAIs, the combinations of Ca:Na formulations with M2 channel inhibitors do not provide an additive or synergistic benefit over each single treatment in vitro.

### Example 13

### Ca:Na Formulations are Effective for Treating Human Parainfluenza Virus 3 (hPIV3)

A cell culture model of human parainfluenza virus 3 (hPIV3) infection was used to study the effects of different nebulized solutions on viral infection. Calu-3 cells were cultured on permeable membranes (12 mm Transwells; 0.4 μm pore size, Corning Lowell, Mass.) until confluent (membrane is fully covered with cells) and air-liquid interface (ALI) cultures were established by removing the apical media and culturing at 37° C./5% CO₂. Cells were cultured for >2 weeks at ALI before each experiment. Normal human bronchial epithelial (NHBE) cells were seeded at passage 2 on permeable membranes (12 mm Millicell, 0.4 μm pore size; Millipore Billerica, Mass.) and incubated (37° C., 5% CO₂, 95% RH) until confluent under liquid-covered culture conditions. Once confluent, the apical media was removed and ALI cultures were established. Cells were cultured for ≧4 weeks ALI prior to each experiment. Prior to each experiment the apical surface of each cell type was washed 3× with PBS. Cells were subsequently exposed to nebulized formulations with an in-house developed Sedimentation chamber and Series 8900 nebulizers (Slater Labs). In experiments performed on Calu-3 cells, Transwells were exposed to nebulized Formulation A, as well as 8:1 Ca:Na formulations (0.5×, 2×, and 8×) (Table 2) in triplicate. The 8:1 ratio of Ca:Na was previously selected as an optimized ratio based on experiments performed with influenza in the same experimental system. Experiments performed on NHBE cells involved exposing Millicells to nebulized 8:1 Ca:Na optimized ratio formulations (0.5×, 2×, and 8×) in duplicate. Immediately after exposure, the basolateral media (media on the bottom side of the Transwell) was replaced with fresh media. Triplicate wells were exposed to each formulation in each test. A second cell culture plate was exposed to the same formulations to quantify the delivery of total salt or calcium to cells. One hour after exposure, cells were infected with 10 μL of hPIV-3 (C242 strain) at a multiplicity of infection of 0.3-0.1 (0.3-0.1 virions per cell). Four hours after aerosol treatment, the apical surfaces were washed to remove excess formulation and unattached virus. The next day (24 hours after formulation exposure) virus released onto the apical surface of infected cells was collected in culture media or PBS and the concentration of virus in the apical wash was quantified by TCID₅₀ (50% Tissue Culture Infectious Dose) assay. The TCID₅₀ assay is a standard endpoint dilution assay that is used to quantify how much of a given virus is present in a sample.

Influenza and hPIV-3 infection was reduced by the Ca:Na formulations in a dose responsive manner (FIG. 21). In both Calu-3 and NHBE cells, treatment with 8× Ca:Na formulation resulted in the greatest decrease in titer compared to the untreated control (p<0.001, compared to respective untreated control; one-way ANOVA with Tukey's multiple comparison post-test), however all three treatments had a significant impact on infection. The 0.5× formulation reduced hPIV3 infection 15.8- and 79.4-fold in Calu-3 and NHBE cells, respectively and the 2× formulation reduced hPIV3 infection 631- and 5011-fold, respectively. The magnitude of these reductions is equivalent or better than that previously seen with human influenza viruses in similar models.

These data extend the findings made with Influenza, and demonstrate that Ca²⁺/Na⁺ formulations are broadly effective at reducing viral infections that result in influenza or influenza-like illness.

### Example 14

Ca:Na formulations at an 8:1 molar ratio of calcium to sodium reduce the infectivity of multiple influenza strains in vitro.

Previous data suggested that Ca:Na formulations at an 8:1 molar ratio reduced the infectivity of multiple H1N1 influenza strains and one H3N2 influenza strain. To extend these findings, we tested two additional H3N2 strains and an influenza B strain. Additionally, we tested an H1N1 strain isolated from swine.

TABLE 10
Influenza strains used in these studies

| Influenza A H1N1 | Influenza A H3N2 | Influenza B |
|---|---|---|
| A/WSN/33 | A/Aichi/2/68 | B/Mass/3/66 |
| A/Puerto Rico/8/34 | A/Panama/2007/99 | |
| A/FM/1/47 | A/Port Chalmers/1/73 | |
| A/Weiss/43 | | |
| A/Swine/IA/40776/92 | | |

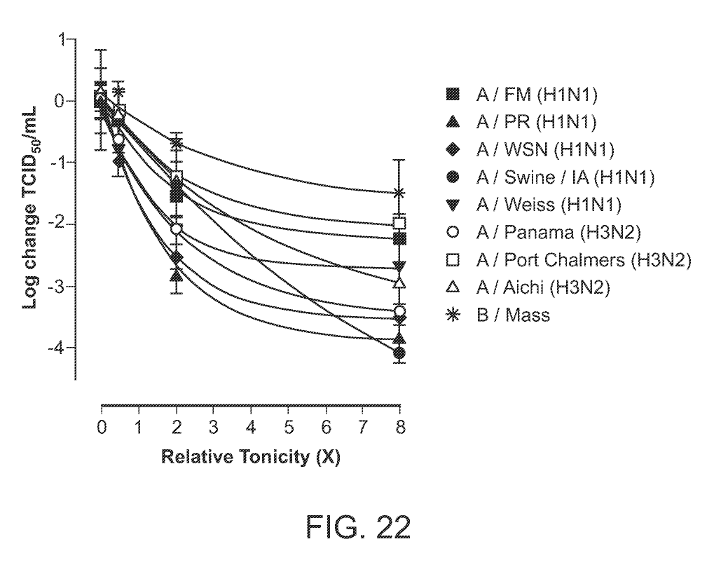

Ca:Na formulations reduced the infectivity of all viruses tested in a dose responsive manner (FIG. 22). The greatest reduction in titer was observed using the 8× formulation, which reduced titers between 32- to 12,589 fold depending on the virus being tested. Thus, optimized ratio formulations comprised of Ca and Na at an 8:1 molar ratio can be effectively used to reduce a broad array of influenza viruses in vitro, suggesting that efficacy in vivo will be independent of influenza strain.

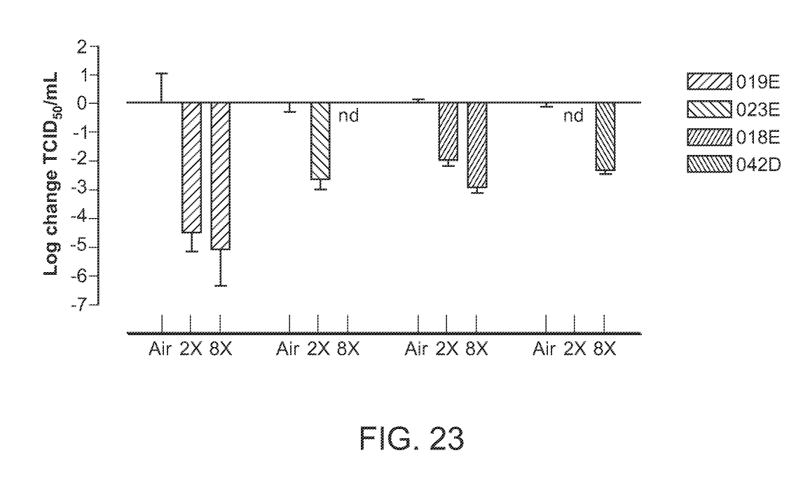

Ca:Na formulations also reduced influenza infection in NHBE cells. To better relate the findings made with Calu-3 cells to the lung, primary normal human bronchial epithelial (NHBE) cell cultures were also tested. These cultures are multicellular and are minimally passaged in vitro prior to testing. To test the effect of Ca:Na formulations (8:1 molar ratio of Ca:Na) at different tonicities, cells were exposed to each formulation and infected with Influenza A/Panama/2007/99. Because NHBEs are primary cultures they are subjected to donor-to-donor variability that is not present in Calu-3 cultures. To account for this variability, NHBE cultures from four different donors were tested. Treatment of NHBE cell cultures resulted in reduced influenza titers in all donors tested, with a maximal reduction of greater than 100-fold in each case (FIG. 23). Thus, the efficacy of Ca:Na formulations extends from Calu-3 cells to primary NHBE cell cultures and further supports the efficacy of these formulations to treat and prevent influenza infection.

The entire teachings of all documents cited herein are hereby incorporated herein by reference.

1. A pharmaceutical composition comprising an effective amount of a calcium salt and an effective amount of an anti-influenza agent, wherein the anti-influenza agent is selected from the group consisting of an NAI, an M2 channel inhibitor, an IMP dehydrogenase inhibitor, an influenza RNA polymerase inhibitor, a sialidase, a sialidase fusion protein, a sialyl multimer or polymer, a siRNA that targets expression of influenza genes, an oligonucleotide that targets expression of influenza genes, interferon alpha, an interferon inducer, and a signal transduction inhibitor, and further wherein the composition is suitable for administration to the respiratory tract and the effective amount of the calcium salt and the effective amount of the anti-influenza agent are synergistic.

2. (canceled)

3. The pharmaceutical composition of claim 2, wherein the anti-influenza agent is an NAI.

4. The pharmaceutical composition of claim 3, wherein the NAI is selected from the group consisting of zanamivir, peramivir, oseltamivir phosphate, oseltamivir carboxylate and combinations thereof.

5. (canceled)

6. The pharmaceutical composition of claim 1, wherein the pharmaceutical composition is a liquid formulation.

7. The pharmaceutical composition of claim 6, wherein the calcium salt is present in a concentration of from about 0.1% to about 20% (w/v).

8. The pharmaceutical composition of claim 1, wherein the pharmaceutical composition is a dry powder.

9. The pharmaceutical composition of claim 8, wherein the calcium salt is present in an amount of from about 30% to about 99% (w/w).

10. The pharmaceutical composition of claim 1, wherein the composition comprises about 1 mg to about 20 mgs of NAI per dose.

11. The pharmaceutical composition of claim 1 further comprising a sodium salt.

12-15. (canceled)

16. The pharmaceutical composition of claim 1, wherein the pharmaceutical composition is formulated to provide an NAI dose of about 1 mg to about 20 mgs to the lungs.

17. (canceled)

18. A method, comprising administering to an individual suspected of having influenza or at risk of having influenza an effective amount of a pharmaceutical composition of claim 1, wherein the pharmaceutical composition is administered to the respiratory tract.

19. A method for treating influenza infection comprising administering to an individual in need thereof an effective amount of a pharmaceutical composition of claim 1, wherein the pharmaceutical composition is administered to the respiratory tract.

20. A method for the prophylaxis of influenza infection comprising administering to an individual at risk for infection by influenza virus an effective amount of a pharmaceutical composition of claim 1, wherein the pharmaceutical composition is administered to the respiratory tract.

21. A method for reducing the spread of influenza infection comprising administering to an individual infected by influenza virus or at risk for infection by influenza virus an effective amount of a pharmaceutical composition of claim 1, wherein the pharmaceutical composition is administered to the respiratory tract.

22-43. (canceled)

44. The pharmaceutical composition of claim 1, where said anti-influenza agent is selected from the group consisting of a NAI, a sialidase and a sialidase fusion protein.

45. (canceled)

46. The method of claim 20, wherein said pharmaceutical composition comprises an anti-influenza agent selected from the group consisting of a NAI, a sialidase, a sialidase fusion protein and combinations thereof.

47-50. (canceled)

51. A method for treating influenza-like illness comprising administering to an individual in need thereof an effective amount of a calcium salt formulation.

52. A method for the prophylaxis of influenza-like illness comprising administering to an individual at risk for infection by influenza-like illness an effective amount of a calcium salt formulation, wherein the calcium salt composition is administered to the respiratory tract.

53-54. (canceled)
